(19) United States
(12) Patent Application Publication
(10) Pub. No.: US 2007/0082939 A1
(43) Pub. Date: Apr. 12, 2007

(54) Methods and compositions for the treatment of neuropathies and related disorders

(76) Inventors: Arnold S. Lippa, Ridgewood, NJ (US); Phil Skolnick, Edgewater, NJ (US); Anthony Basile, Hoboken, NJ (US); Zhengming Chen, Belle Meade, NJ (US); Joseph W. Epstein, Monroe, NY (US)

Correspondence Address: BLACK LOWE & GRAHAM PLLC, 701 Fifth Avenue - Suite 4800, Seattle, WA, 98104, US

(21) Appl. No.: 11/492,608

(22) Filed: Jul. 24, 2006

Related U.S. Application Data
(60) Provisional application No. 60/702,800, filed on Jul. 26, 2005.

(51) Int. Cl. A61K 31/403

(52) U.S. Cl. 514/412

(57) ABSTRACT

The present invention provides novel compositions and methods for treating symptoms associated with neuropathic disorders such as hyperalgesia, allodynia, and parasthesias, using a 1-aryl-3-azabicyclo[3.1.0] hexane. The invention further relates to the use of 1-aryl-3-azabicyclo[3.1.0] hexanes in pharmaceutical compositions and methods for treating neuropathic disorders and related symptoms in mammals. Patients amenable to treatment according to the invention include those suffering from diabetic neuropathies, post-herpetic neuralgia, trigeminal neuralgia, chronic lower back pain, sciatica, idiopathic and post-traumatic neuropathies, HIV-associated neuropathic pain, among many other neuropathic disorders and related symptoms.

### CROSS REFERENCE TO RELATED APPLICATIONS

This application claims the priority benefit of copending U.S. Provisional Patent Application No. 60/702,800, filed Jul. 26, 2005.

### TECHNICAL FIELD

The present invention relates to compositions and methods for treating neuropathic disorders and symptoms associated therewith, including neuropathic pain.

### BACKGROUND OF THE INVENTION

Neuropathic disorders are frequently complex in their etiology, and individuals suffering from neuropathy often present with multiple and variable adverse symptoms. Among the most common and severe adverse symptoms that attend neuropathic disorders is a syndrome commonly referred to as “neuropathic pain.” Neuropathic pain is characterized and distinguished from acute, nociceptive pain (for example, pain caused by a burn or surgical incision) by distinct neurological and sensory features that render its treatment refractory to standard treatments for nociceptive pain.

The distinct neurological and sensory features that characterize neuropathic disorders variably include allodynia (a painful response to non-noxious stimuli, such as the touch of clothing), hyperalgesia (a heightened or extreme sensitivity to painful stimuli), paraesthesias (abnormal sensations such as tingling, burning, pricking or tickling); hyperesthesia (enhanced sensitivity to natural stimuli); and dysesthesias (disagreeable sensations produced by ordinary stimuli). These diverse “pain” symptoms are differentially clustered among patients with neuropathic disorders, and may be continuous or paroxysmal in presentation.

Neuropathic disorders are most commonly attributable to injury or pathogenesis directly or indirectly affecting the peripheral and/or central nervous system. By virtue of these underlying pathogenic changes, which can be produced by diverse insults, ranging from viral infection of the central nervous system to amputation of a limb, neuropathies are commonly associated with aberrant somatosensory processing in the peripheral and/or central nervous system. The attendant sensory symptoms of neuropathy are typically qualitatively distinct from nociceptive pain, producing exaggerated or inappropriate responses to stimuli, or distinct sensations such as burning, shooting, tingling, piercing, lacerating, or electric shock-like sensory responses. These adverse sensory conditions are commonly referred to as “neuropathic pain”, but are distinct neuropathic symptoms as compared to nociceptive pain.

Neuropathic disorders can be accompanied by a host of comorbid symptoms apart from neuropathic pain—for example depression, insomnia, fatigue, mood disorders, post-traumatic stress, withdrawal, and/or loss of mental and/or physical function. Other symptoms of neuropathic disorders, which may be causal to, evoked by, or secondary to, neuropathic pain, include somatic stress symptoms, such as increased blood pressure, heart rate, and respiration.

Another distinguishing aspect of neuropathic disorders is the chronic nature of attendant symptoms, which frequently persist for many weeks, up to 3-6 months, or longer. Neuropathic conditions thus impose an enduring loss of quality and function in the lives of sufferers. In addition to chronic neuropathic pain, various chronic secondary impacts are well documented for neuropathy patients—including increased risk of heart disease, lowered immunity, increased risk of illness, and lasting psychological disorders. Guarding and disuse of painful body parts also frequently occurs in neuropathy patients, which can lead to other adverse consequences such as muscle weakness or atrophy, muscle tightness or spasm, shortening or loss of elasticity of tendons and ligaments and associated loss of function (e.g., reduced range-of-motion), and weakening of bones associated with increased fracture risk.

While there can be many underlying causes for neuropathies, they are most often triggered by direct injury or damage to the peripheral and/or central nervous system. Exemplary forms of neuropathy, and related disorders and symptoms associated with neuropathies, include, diabetic neuropathy; peripheral neuropathy; distal symmetrical polyneuropathy; post-herpetic neuralgia; trigeminal neuralgia; alcoholism-related neuropathy; HIV sensory neuropathy; sciatica; spinal cord injury; post-stroke neuropathy; multiple sclerosis; Parkinson's disease; idiopathic or post-traumatic neuropathy; mononeuritis; cancer-associated neuropathy; peripheral nerve trauma; nerve transection; carpal tunnel injury; certain forms of chronic lower back pain, neuropathy associated with Fabry's disease; vasculitic neuropathy; neuropathy associated with Guillain-Barre syndrome; and entrapment neuropathy. Across this broad spectrum, neuropathies affect a vast number of patients worldwide, and result in billions of dollars of annual costs for health care and lost productivity. For example, among the estimated 150 million people with diabetes worldwide, diabetic neuropathy affects up to 50% of this large patient population.

Although neuropathic symptoms are most often triggered by injury, the precipitating injury need not involve direct damage to the nervous system. In many cases, precipitating factors of neuropathies are indirect—for example, nerves can be infiltrated or compressed by tumors, strangulated by scar tissue, or inflamed by infection. Thus, a number of indirect injuries, diseases and conditions can result in neuropathic disorders, including: dietary or absorption abnormality; vitamin deficiencies; heavy metal poisoning; complex regional pain syndrome; fibromyalgia; Wallenberg's syndrome; connective tissue disease; plexus irradiation; ischemic irradiation; hematomyelia; dyscraphism; tumor compression; arteriovenuous malformation; syphilitic myelitis; commissural myelotomy; arachnoiditis; root avulsion; prolapsed disk compression; lumbar and cervical pain; reflex sympathic dystrophy; postthoracotomy pain; postmastectomy pain; phantom limb syndrome; and various other chronic pain syndromes.

It is widely understood that neuropathic pain represents a distinct pain phenomenon from ordinary pain (i.e., normal, adaptive pain responses classified as nociceptive, or systemic, pain). Although neuropathic and nociceptive pain may share some common features, their differential diagnosis and treatment is well recognized. In addition to the distinguishing features noted above, nociceptive pain typically arises from acute trauma (for example, sprains, bone fractures, torn ligaments, burns, and cuts), occurring in or near damaged tissues, and usually resolves once the causal injury abates and damaged tissues heal. Nociceptive pain therefore typically comprises acute pain symptoms mediated by nocioceptors—sensory neurons that respond to stimuli associated with tissue injury. Nociceptive pain is also generally self-limiting and serves to protect biological function by signaling current tissue insult or damage. In contrast, neuropathic pain and other related symptoms of neuropathy typically persist for months, or even years—far beyond the apparent healing of damaged tissues.

The chronic nature of most neuropathic disorders greatly complicates treatment. Among the most significant complications in this context is the requirement for long-term medication or other intervention to treat and manage neuropathic disorders and related symptoms, including neuropathic pain.

Current drug therapies for neuropathic disorders are seriously limited in terms of drug selection, efficacy, and side effects. Currently-practiced treatments for neuropathic disorders include the use of a variety of compounds with diverse mechanisms of activity, such as amitriptyline, carbamazepine, phenytoin, mexiletin, neurontin, gabapentin, and duloxetine. These and other drugs currently employed for neuropathic treatments frequently provide low efficacy for treating symptoms of neuropathies, and are commonly associated with adverse side-effects. The efficacy/safety profiles of current neuropathy drugs may especially problematic in the instance of long-term use, as is typically necessary to manage neuropathy symptoms.

More invasive treatments for neuropathic disorders include epidural spinal cord stimulation; deep brain stimulation; neurectomy; and rhizotomy. Each of these methods has been tried for neuropathy patients with limited success, sometimes resulting in increased pain, for example due to deafferentation.

The available armamentarium of drugs for treating neuropathic disorders is fundamentally distinct from the host of analgesics and other compounds ordinarily used to treat nociceptive pain. The diverse assemblage of drugs used for managing symptoms of neuropathies are not generally prescribed, nor recognized as effective, in the treatment of nociceptive pain. Likewise, whereas nociceptive pain generally responds well to opioids and other conventional analgesics, such as non-steroidal anti-inflammatories (NSAIDS) and COX-2 inhibitors, neuropathic pain and other symptoms of neuropathy are generally unresponsive, or insufficiently responsive, to these conventional drug regimens for treating nociceptive pain. The refractory nature of neuropathic disorders to treatment using NSAIDS, (e.g., ibuprofen, acetaminophen, aspirin, and celecoxibid) and opioids (e.g., morphine, oxymorphone, and codeine) is well documented (Max, et al., Clin. And Pharm. Therapy, 43:363; Max, et al., Neurology 38:1427, (1988));

In view of the foregoing, there is an important, unmet need in the art for alternative compositions and methods for treating neuropathic disorders and related adverse conditions and symptoms, including neuropathic pain.

### SUMMARY OF EXEMPLARY EMBODIMENTS OF THE INVENTION

It is therefore an object of the present invention to provide novel and improved compositions and methods for treating and preventing neuropathic disorders and related conditions in mammalian subjects.

The invention achieves these objects and satisfies additional objects and advantages by providing new and surprisingly effective compositions and methods for treating neuropathies and related symptoms attendant to neuropathic disorders including, but not limited to, paraesthesias, allodynia, hyperalgesia and other sensory symptoms of neuropathies often referred to as neuropathic pain, in mammals. The subject methods and compositions are directed to procedures, compounds, or formulations that employ an effective amount of a 1-aryl-3-azabicyclo[3.1.0]hexane sufficient to alleviate one or more symptoms of neuropathy in a mammalian subject.

In more detailed embodiments, the compositions and methods of the invention for treating neuropathic disorders employ an effective amount of a compound or formulation comprising a 1-aryl-3-azabicyclo[3.1.0]hexane having at least one substituent on the phenyl/aryl ring.

In exemplary embodiments, methods and compositions of the invention for treating neuropathic disorders and related symptoms employ a novel 1-aryl-3-azabicyclo[3.1.0]hexane having at least one substitution on the aryl ring and characterized, at least in part, by formula I, below:

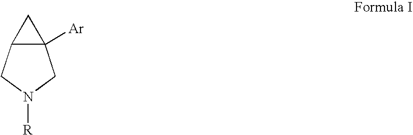

wherein Ar is a phenyl or other aromatic group having at least one substitution on the aryl ring, and wherein R is selected from, for example, hydrogen, C₁₋₆ alkyl, halo(C₁₋₆)alkyl, C₃₋₉ cycloalkyl, C₁₋₅ alkoxy(C₁₋₆)alkyl, carboxy(C₁₋₃)alkyl, C₁₋₃ alkanoyl, carbamate, halo(C₁₋₃)alkoxy(C₁₋₆)alkyl, C₁₋₃ alkylamino(C₁₋₆)alkyl, and di(C₁₋₃)alkylamino(C₁₋₆)alkyl, cyano(C₁₋₆)alkyl, methyl, ethyl, trifluoromethyl, trifluoroethyl and 2-methoxyethyl.

In certain detailed embodiments, the compounds and formulations of the invention for treating neuropathic disorders and/or related symptoms comprise a 1-aryl-3-azabicyclo[3.1.0]hexane having an aza substitution in place of the hydrogen associated with the nitrogen at the ‘3’ position.

In other detailed embodiments of the invention, the compounds and formulations of the invention for treating neuropathic disorders and/or related symptoms comprise a 1-aryl-3-azabicyclo[3.1.0]hexane having at least one substitution on the aryl ring, as well as an aza substitution on the nitrogen at the ‘3’ position.

In additional detailed embodiments, the compounds and formulations of the invention for treating neuropathic disorders and/or related symptoms comprise a 1-aryl-3-azabicyclo[3.1.0]hexane having two or more substituents on the phenyl/aryl ring.

In other detailed embodiments, the compounds and formulations of the invention for treating neuropathic disorders and/or related symptoms comprise a 1-aryl-3-azabicyclo[3.1.0]hexane having multiple substitutions on the aryl ring, combined with an “aza substitution” on the nitrogen at the ‘3’ position.

Mammalian subjects amenable for treatment using the methods and compositions of the invention include, but are not limited to, human and other mammalian subjects suffering from neuropathic pain syndromes and/or presenting with one or more neuropathic pain-related symptoms. Subjects within these target groups for treatment include, for example, patients presenting with neuropathic pain associated with diabetic neuropathy, diabetic peripheral neuropathy, distal symmetrical polyneuropathy, post-herpetic neuralgia, trigeminal neuralgia, pain secondary to alcoholism, sciatica, post-stroke pain, multiple sclerosis, shingles, idiopathic or post-traumatic neuropathy and mononeuritis, HIV-associated neuropathic pain, cancer, carpal tunnel syndrome, neuropathy associated with Fabry's disease, vasculitic neuropathy, neuropathy associated with Guillain-Barre syndrome, dietary or absorption abnormality, spinal cord injury, chronic lower back pain, iatrogenic-induced neuropathies, vitamin deficiencies, heavy metal poisoning, complex regional pain syndrome, fibromyalgia, peripheral nerve trauma, entrapment neuropathy, nerve transection, Wallenberg's syndrome, connective tissue disease, plexus irradiation, ischemic irradiation, hematomyelia, dyscraphism, tumor compression, arteriovenuous malformation, syphilitic myelitis, commissural myelotomy, arachnoiditis, root avulsion, prolapsed disk compression, lumbar and cervical pain, reflex sympathic dystrophy, phantom limb syndrome and other chronic and debilitating pain syndromes.

These and other subjects are effectively treated by administering to the subject an effective amount of a 1-aryl-3-azabicyclo[3.1.0]hexane to alleviate one or more symptom(s) of a neuropathic disorder in the subject. The therapeutic methods and formulations of the invention may employ 1-aryl-3-azabicyclo[3.1.0]hexanes in a variety of forms including pharmaceutically acceptable salts, enantiomers, polymorphs, solvates, hydrates and/or prodrugs or combinations thereof.

Within additional aspects of the invention, combinatorial formulations and methods are provided which employ an effective amount of a 1-aryl-3-azabicyclo[3.1.0]hexane and one or more additional active agents, that are combinatorially formulated or coordinately administered with the 1-aryl-3-azabicyclo[3.1.0]hexane, or one or more coordinate, non-drug treatment method coordinately administered with the 1-aryl-3-azabicyclo[3.1.0]hexane, to alleviate one or more symptoms associated with a neuropathic disorder in a mammalian subject. Exemplary combinatorial formulations and coordinate treatment methods in this context employ a 1-aryl-3-azabicyclo[3.1.0]hexane in combination with one or more conventional drugs or non-drug treatment methods for treating symptoms attendant to neuropathic disorders, including, but not limited, to: amitriptyline; carbamazepine; phenytoin; mexiletine; neurontin; gabapentin; duloxetine; baclofen; tramadol; antiarrhythmics; antiepileptics; anti-convulsants; capsaicin cream; membrane-stabilizing drugs; N-methyl-D-aspartate receptor (NMDA) antagonists; surgery; transcutaneous electrical nerve stimulation; epidural spinal cord stimulation; neurectomy; rhizotomy; dorsal root entry zone lesion; cordotomy; thalamotomy; and neuroablation.

The forgoing objects and additional objects, features, aspects and advantages of the present invention are further exemplified and described in the following detailed description.

### BRIEF DESCRIPTION OF DRAWINGS

FIG. 1, Panel A, is a graph of experimental results demonstrating that (±)-1-(4-methylphenyl)-3-azabicyclo[3.1.0]hexane hydrochloride (bicifadine HCl) suppresses mechanical hyperalgesia in the Chung model of neuropathic pain. Bicfifadine is as effective as a near lethal dose morphine in blocking mechanical hyperalgesia. The efficacy of bicifadine for treating neuropathic symptoms in this model is specific, as indicated by the data showing that bicifadine had no effect on the pain threshold in the non-lesioned paw (Panel B). *, **; Significantly different from vehicle group, P<0.05, 0.01, respectively, Student's t-test.

FIG. 2, Panel A, is a graph of experimental results demonstrating that (±)-1-(4-methylphenyl)-3-azabicyclo[3.1.0]hexane hydrochloride (bicifadine HCl) suppresses thermal hyperalgesia in the Chung model of neuropathic pain. Bicfifadine is as effective as a near lethal dose morphine in blocking mechanical hyperalgesia. The efficacy of bicifadine for treating neuropathic symptoms in this model is specific, as indicated by the data showing that bicifadine had no effect on the pain threshold in the non-lesioned paw (Panel B). *, **; Significantly different from vehicle group, P<0.05, 0.01, respectively, Student's t-test.

FIG. 3, Panel A, is a graph of experimental results demonstrating that 1-(3,4-dichlorophenyl)-3-azabicyclo[3.1.0]hexane suppresses mechanical hyperalgesia in the Chung model of neuropathic pain. The efficacy of 1-(3,4-dichlorophenyl)-3-azabicyclo[3.1.0]hexane for treating neuropathic symptoms in this model is specific, as indicated by the data showing that the compound had no effect on the pain threshold in the non-lesioned paw (Panel B). *, **; Significantly different from vehicle group, P<0.05, 0.01, respectively, Student's t-test.

FIG. 4, Panel A, is a graph of experimental results demonstrating that 1-(3,4-dichlorophenyl)-3-azabicyclo[3.1.0]hexane suppresses thermal hyperalgesia in the Chung model of neuropathic pain. The efficacy of 1-(3,4-dichlorophenyl)-3-azabicyclo[3.1.0]hexane for treating neuropathic symptoms in this model is specific, as indicated by the data showing that the compound had no effect on the pain threshold in the non-lesioned paw (Panel B). *, **; Significantly different from vehicle group, P<0.05, 0.01, respectively, Student's t-test.

FIG. 5 is a graph of experimental results demonstrating that bicifadine effectively alleviates neuropathy symptoms (mechanical hyperalgesia) in the Streptozotocin (STZ)-induced diabetes rat model of neuropathy. *, Significantly different from vehicle group, P<0.05, Student's t-test.

### DETAILED DESCRIPTION OF THE EXEMPLARY EMBODIMENTS OF THE INVENTION

The present invention provides novel compositions and methods for treating and/or preventing symptoms associated with neuropathic disorders in mammalian subjects, including humans. The therapeutic and prophylactic formulations and methods of the invention employ an effective amount of a 1-aryl-3-azabicyclo[3.1.0]hexane, which, when administered to a mammalian subject, effectively treats or prevents a neuropathic disorder, one or more symptom(s) or condition(s) of a neuropathic disorder, in the subject.

In various embodiments, the methods and compositions of the invention employ one or more aryl-substituted, and/or aza-substituted, 1-aryl-3-azabicyclo[3.1.0]hexanes characterized, at least in part, by formula I, below: 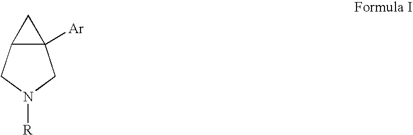
wherein Ar is a phenyl or other aryl group, optionally having at least one substitution on the aryl ring, and wherein R is H or an optional aza substituent selected from, for example, hydrogen, C₁₋₆ alkyl, halo(C₁₋₆)alkyl, C₃₋₉ cycloalkyl, C₁₋₅ alkoxy(C₁₋₆)alkyl, carboxy(C₁₋₃)alkyl, C₁₋₃ alkanoyl, carbamate, halo(C₁₋₃)alkoxy(C₁₋₆)alkyl, C₁₋₃ alkylamino(C₁₋₆)alkyl, and di(C₁₋₃)alkylamino(C₁₋₆)alkyl, cyano(C₁₋₆)alkyl, methyl, ethyl, trifluoromethyl, trifluoroethyl and 2-methoxyethyl.

As used herein, the structural designation “Ar” represents a phenyl or other aromatic group. An aromatic group designates cyclically conjugated systems of 4n+2 electrons, that is with 6, 10, 14 etc. π-electrons; a monocyclic, bicyclic or tricyclic saturated heterocycle represents a ring system consisting of 1, 2 or 3 rings and comprising at least one heteroatom selected from O, N or S, said ring system containing only single bonds; a monocyclic, bicyclic or tricyclic partially saturated heterocycle represents a ring system consisting of 1, 2 or 3 rings and comprising at least one heteroatom selected from O, N or S, and at least one double bond provided that the ring system is not an aromatic ring system; a monocyclic, bicyclic or tricyclic aromatic heterocycle represents an aromatic ring system consisting of 1, 2 or 3 rings and comprising at least one heteroatom selected from O, N or S. The term “phenyl” as used herein refers to a monocyclic carbocyclic ring system having one aromatic ring. The phenyl group can also be fused to a cyclohexane or cyclopentane ring. The phenyl and aromatic groups of this invention can be optionally substituted.

An illustrative assemblage of aryl substituted 1-aryl-3-azabicyclo[3.1.0]hexanes for use within this aspect of the invention is provided in Table 1, below. In each of these exemplary compounds, there is no aza substituent (i.e., the hydrogen associated with the nitrogen at the ‘3’ position has been retained), however it is further contemplated that the exemplified aryl substitutions can be combined with aza substitutions as described below to yield “bisubstituted” compounds as candidates for treating neuorpathic disorders and related symptoms as described herein.

TABLE 1
Exemplary Aryl-Substituted 1-aryl-3-azabicyclo[3.1.0] hexanes

| | |
|---|---|
| 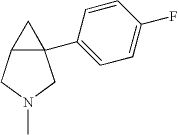 | 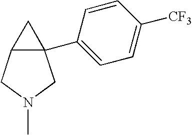 |
| 1-(4-fluorophenyl)-3-methyl-3-aza- | 3-methyl-1-(4-(trifluoromethyl) |
| bicyclo[3.1.0]hexane | phenyl)-3-aza-bicyclo[3.1.0]hexane |
| 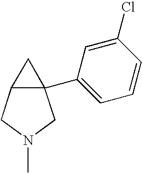 | 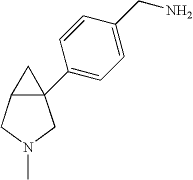 |
| 1-(3-chlorophenyl)-3-methyl-3-aza- | (4-(3-methyl-3-aza-bicyclo[3.1.0] |
| bicyclo[3.1.0]hexane | hexan-1-yl)phenyl)methanamine |

In certain exemplary embodiments of the invention, the effective compositions and methods for treating neuropathies and related symptoms employ an aryl-substituted, 1-aryl-3-azabicyclo[3.1.0]hexane selected from (±)-1-(4-methylphenyl)-3-azabicyclo[3.1.0]hexane hydrochloride (bicifadine HCl), enantiomers of bicifadine, other salts of bicifadine, prodrugs of bicifadine, polymorphs, hydrates, and solvates of bicifadine, or any combination of the foregoing forms of bicifadine. In more detailed embodiments, bicifadine hydrochloride is employed within the therapeutic formulations and methods of the invention. Bicifadine HCl, ((±)-1-(4-methylphenyl)-3-azabicyclo[3.1.0]hexane hydrochloride; also referred to as racemic 1-(p-toyl)-3-azabicyclo[3.1.0]hexane hydrochloride), is described as a non-narcotic analgesic in U.S. Pat. No. 4,231,935 and U.S. Pat. No. 4,196,120 (each incorporated herein by reference). Bicifadine, represented (as the free base) by the structural formula II, below, has been reported to be potent and active in the “Randall-Selitto” test, an animal model of acute inflammatory pain (see, e.g., Epstein et al., J. Med. Chem. 24(5):481, 1981; Epstein et al., NIDA Res. Monogr. pp. 93-98, 1982). Both opiates (e.g., morphine and codeine) and NSAIDs (e.g., aspirin), compounds used to treat acute pain, are also active in this model. In this model, inflammatory pain is produced by injection of yeast extract into the plantar surface of the rat paw. Consistent with these studies, bicifadine has been confirmed to have analgesic action for treating nociceptive pain in humans. In particular, bicifadine has been reported to be as effective as codeine and tramadol, two commonly used analgesics for treating nociceptive pain, in relieving pain following dental surgery (Czobor P., et al., 2003); (Czobor P., et al., 2004).

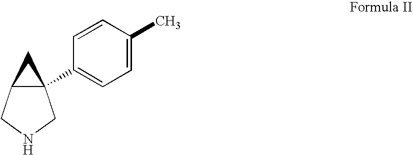

Bicifadine HCl also exists in at least two polymorphic crystalline forms, designated polymorph forms A and B (e.g., as described in U.S. patent application Ser. No. 10/702,397, herein incorporated by reference). Other polymorphic forms of bicifadine hydrochloride may exist and are likewise candidates for use within the methods and compositions of the invention for treating a neuropathic disorder and/or related symptom(s).

Polymorphs include compounds with identical chemical structure but different internal structures. Additionally, many pharmacologically active organic compounds regularly crystallize incorporating second, foreign molecules, especially solvent molecules, into the crystal structure of the principal pharmacologically active compound forming pseudopolymorphs. When the second molecule is a solvent molecule, the pseudopolymorphs can also be referred to as solvates. All of these additional forms of bicifadine are likewise useful within the anti-incontinence methods and formulations of the invention.

Polymorph form A of bicifadine HCl can be formed, for example, by methods disclosed in U.S. Pat. No. 4,231,935 and U.S. Pat. No. 4,196,120 (each of which is incorporated herein by reference). Polymorph form B can be formed, for example, by methods disclosed in U.S. patent application Ser. No. 10/702,397, related international application PCT/US2003/035099 (Intl. Pub. No. WO04/043920), and priority U.S. Provisional Patent Application No. 60/424,982 (each incorporated by reference). For example, polymorph B can be formed from polymorph form A through the application of kinetic energy and through crystallization techniques. In one embodiment, kinetic energy in the form of agitating, stirring, grinding or milling can be applied to a pure composition of polymorph form A, or a mixture of forms A and B, particularly at selected temperatures, for example from about −200° C. to about 50° C., in another embodiment from about −200° C. to about 35° C., in a further embodiment from about −200° C. to about 0° C. In another embodiment, polymorph B can be crystallized from a solution of polymorph A that is heated and allowed to cool under defined conditions of temperature and time to form polymorph B. Under selected conditions, preparations of pure polymorph A of bicifadine, or mixtures of polymorph A and B of bicifadine, can be processed to yield desired compositions containing enriched quantities of polymorph B, for example ranging from approximately at least 10%, to about 10-20%, 20-35%, 35-50%, 50-70%, 70-85%, 85-95%, and up to 95-99% or greater (by weight) bicifadine polymorph B in the composition.

The polymorphs of bicifadine HCl may be characterized by their infrared spectra and/or their x-ray powder diffraction pattern. As described in U.S. patent application Ser. No. 10/702,397 incorporated above, X-ray powder diffraction (XRPD) analyses of polymorph forms A and B of racemic bicifadine hydrochloride were performed with a Shimadzu XRD-6000 X-ray powder diffractometer using Cu Ka radiation. The bicifadine was loaded onto the machine as a crystalline powder. The instrument was equipped with a fine focus X-ray tube. The tube voltage and amperage were set to 40 kV and 40 mA, respectively. The divergence and scattering slits were set at 1° and the receiving slit was set at 0.15 mm. Diffracted radiation was detected by a NaI scintillation detector. A theta-to theta continuous scan at 3/min (0.4 sec/0.02° step) from 2.5 to 40°2θ was used. A silicon standard was analyzed to check the instrument alignment. Data were collected and analyzed using XRD-6000 v.4.1.

The X-ray powder diffraction pattern of polymorph form A of racemic bicifadine hydrochloride is given in terms of “d” spacing and relative intensities (I) is as follows (s=strong, m=medium, w=weak, v=very, d=diffuse) and these terms are set forth in Table 2 below, and the X-ray powder diffraction pattern of form B of bicifadine hydrochloride is set forth in Table 3 below:

TABLE 2
Peak Positions, d-Spacings, and Intensities for Polymorph Form
A Bicifadine Hydrochloride

| 2θ (deg) | d (Å) | I^{a} |
|---|---|---|
| 5.35 | 16.50 | Vs |
| 10.61 | 8.33 | Vs |
| 11.45 | 7.72 | W |
| 15.22 | 5.82 | W |
| 15.93 | 5.56 | W |
| 16.97 | 5.22 | W |
| 18.37 | 4.83 | W |
| 20.04 | 4.43 | Md |
| 20.26 | 4.38 | Md |
| 21.22 | 4.18 | M |
| 21.89 | 4.06 | S |
| 23.12 | 3.84 | Md |
| 23.54 | 3.78 | Wd |
| 26.63 | 3.34 | M |
| 27.83 | 3.20 | Wd |
| 28.32 | 3.15 | Wd |
| 30.67 | 2.91 | Wd |
| 32.03 | 2.79 | S |
| 37.57 | 2.39 | W |
| 38.20 | 2.35 | W |

^{a}s = strong, m = medium, w = weak, v = very, d = diffuse

TABLE 3
Peak Positions, d-Spacings, and Intensities for Polymorph Form
B Bicifadine Hydrochloride

| 2θ (deg) | d (Å) | I^{a} |
|---|---|---|
| 5.08 | 17.39 | Vs |
| 10.07 | 8.77 | S |
| 15.19 | 5.83 | S |
| 16.83 | 5.27 | S |
| 18.64 | 4.76 | Md |
| 18.76 | 4.73 | Md |
| 19.64 | 4.52 | W |
| 20.16 | 4.40 | M |
| 21.96 | 4.05 | M |
| 22.37 | 3.97 | S |
| 23.16 | 3.84 | W |
| 24.00 | 3.70 | W |
| 25.27 | 3.52 | D |
| 27.33 | 3.26 | Md |
| 27.74 | 3.21 | M |
| 29.00 | 3.08 | M |
| 30.43 | 2.93 | Md |
| 31.84 | 2.80 | Wd |
| 32.29 | 2.77 | W |
| 35.27 | 2.54 | Wd |
| 35.64 | 2.52 | W |

^{a}s = strong, m = medium, w = weak, v = very, d = diffuse

Table 2 and Table 3 represent the XRPD pattern of the peak positions of bicifadine hydrochloride form A and form B respectively having reduced particle size. The results in these tables demonstrate the difference between the XRPD patterns of form A and form B at a reduced particle size. However, there are key peaks at given angles in this pattern which identify polymorph form B of bicifadine hydrochloride and are typically present in the XRPD pattern of polymorph form B irrespective of its particle size. These angles, expressed as 2θ (deg), locating these major peaks, which alone or in any distinguishing combination, distinguish bicifadine polymorph form B from form A, using Cu Ka radiation, are: 5.08; 10.07; 20.16; 25.17; and 30.43.

The infrared spectra were obtained for each of the samples using a Magna-IR 860® Fourier transform infrared (FT-IR) spectrophotometer (Thomas Nicolet) equipped with an Ever-Glo mid/far IR source, an extended range potassium bromide (KBr) beamsplitter, and a deuterated triglycine sulfate (DTGS) detector. The spectrophotometer measured the intensity of infrared light bands of each of the samples at given wavelengths. A diffuse reflectance accessory (the Collector™, Thermo Spectra-Tech) was used for sampling. Each spectrum represents 256 co-added scans collected from 400-4000 cm⁻¹ at a spectral resolution of 4 cm⁻¹. Sample preparation consisted of placing the sample of powder containing crystals in either polymorph form A or form B into a 13-mm diameter cup and leveling the material with a frosted glass slide. A background data set was acquired with an alignment mirror in place. The reflectance R is the ratio, at a given wavenumber, of the light intensity of the sample/light intensity of the background set. A Log 1/R(R=reflectance) spectrum acquired by taking a ratio of these two data sets (the sample and the background light intensities) against each other. The infrared spectrum of polymorph A or racemic bicifadine hydrochloride as a dry crystalline powder, as provided in Table 4, showed the indicated main peaks which characterized this polymorph. The infrared spectrum of polymorph B of racemic bicifadine hydrochloride in dry crystalline powder, as provided in Table 5, showed the indicated main peaks which characterize this polymorph.

TABLE 4
Infrared Peak Positions For Polymorph Form A Bicifadine Hydrochloride.
All values in wavenumbers (cm⁻¹)

3949
2923
2431
2280
2091
1895
1790
1595
1522
1430
1376
1233
1130
1088
1068
1050
900
825
781
714
689
652
574
533
437

TABLE 5
Infrared Peak Positions for Polymorph Form B Bicifadine Hydrochloride.
All values in wavenumbers (cm⁻¹)

3185
2769
2437
2276
2108
1908
1804
1658
1596
1518
1453
1403
1343
1305
1274
1209
1131
1111
1022
963
904
891
856
818
783
719
684
660
637
580
532
475
422

Table 4 and Table 5 provide the complete patterns of the infrared peak positions with respect to polymorph form A and polymorph form B of bicifadine hydrochloride respectively. However, there are certain key peaks, within this pattern, which are associated with polymorph form B of bicifadine hydrochloride and are sufficient to characterize this polymorph, individually or in any distinguishing combination. These peaks, expressed in wavenumbers (cm⁻¹), are: 2108; 891; 856; 719; and 660.

Bicifadine formulations for treating neuropathies and related symptoms within the invention may comprise any crystalline polymorphic or amorphous form of the compound, or mixture(s) thereof. In exemplary embodiments, effective therapeutic dosage forms of treating mammalian subjects presenting with a neuropathic disorder will comprise essentially pure bicifadine HCl polymorph “form A” (i.e., having a concentration of 90-95% form A by weight of total bicifadine present), essentially pure “form B”, or any mixture of polymorph forms A and B. In certain embodiments, the composition may contain from about 10% to 98% polymorph form B. In other embodiments there may be present in the formulation greater than about 50% polymorph form B, greater than about 75% polymorph B, or greater than about 90% polymorph B.

In additional embodiments, one or more isolated (+) or (−) enantiomers of bicifadine are employed within the methods and compositions of the invention for treating neuropathies and related symptoms. The (+) and (−) enantiomers of bicifadine, and methods for resolving these enantiomers to yield essentially pure compositions of the respective enantiomers, are reported by Epstein et al. (J. Med. Chem. 24(5):481, 1981; NIDA Res. Monour. pp. 93-98, 1982). See, also U.S. Pat. No. 4,131,611; U.S. Pat. No. 4,118,417; U.S. Pat. No. 4,196,120; U.S. Pat. No. 4,231,935; and U.S. Pat. No. 4,435,419, each incorporated herein by reference). In exemplary embodiments, effective therapeutic dosage forms for treating mammalian subjects presenting with a neuropathic disorder will comprise essentially pure (+) bicifadine (i.e., having a concentration of 90-95% of the (+) enantiomer by weight of total bicifadine present), essentially pure (−) bicifadine, or any racemic mixture of the (+) and (−) enantiomeric forms of bicifadine. In certain embodiments, the composition may contain from about 10% to 98% (+) or (−) bicifadine. In other embodiments there may be present in the formulation greater than about 50% (+) or (−) bicifadine, greater than about 75% (+) or (−) bicifadine, or greater than about 90% (+) or (−) bicifadine.

In other detailed embodiments, the compositions and methods of the invention for treating neuropathic disorders and/or related symptoms employ a 1-aryl-3-azabicyclo[3.1.0]hexane that has an “aza” substitution on the nitrogen at the ‘3’ position. In related embodiments, a bi-substituted 1-aryl-3-azabicyclo[3.1.0]hexane is featured in the subject compositions and methods that has at least one substitution on the aryl ring, and a further, aza substitution on the nitrogen at the ‘3’ position. As used herein the terms “aza substitution” and “aza-substituted” refer to 1-aryl-3-azabicyclo[3.1.0]hexanes wherein a hydrogen normally associated with the nitrogen at the ‘3’ position has been replaced with a different aza substituent, as exemplified herein below.

Within exemplary compositions and methods for treating neuropathic disorders and/or related symptoms employing “bi-substituted” 1-aryl-3-azabicyclo[3.1.0]hexanes, the compositions and methods contain or employ such compounds having at least one substitution on the aryl ring, as well as an aza-subsitution, i.e., as characterized in part by formula III, below: 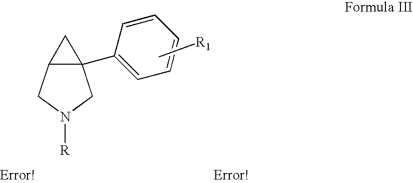
wherein R is selected from, for example, C₁₋₆ alkyl, halo(C₁₋₆)alkyl, C₃₋₉ cycloalkyl, C₁₋₅ alkoxy(C₁₋₆)alkyl, carboxy(C₁₋₃)alkyl, C₁₋₃ alkanoyl, carbamate, halo(C₁₋₃)alkoxy(C₁₋₆)alkyl, C₁₋₃ alkylamino(C₁₋₆)alkyl, and di(C₁₋₃)alkylamino(C₁₋₆)alkyl, cyano(C₁₋₆)alkyl, methyl, ethyl, trifluoromethyl, trifluoroethyl and 2-methoxyethyl; and wherein R₁ is selected from, for example, halogen, C₁₋₃ alkyl, C₂₋₄ alkenyl, C₂₋₄ alkynyl, halo(C₁₋₃)alkyl, cyano, hydroxy, C₃₋₅ cycloalkyl, C₁₋₃ alkoxy, C₁₋₃ alkoxy(C₁₋₃)alkyl, carboxy(C₁₋₃)alkyl, C₁₋₃ alkanoyl, halo(C₁₋₃)alkoxy, nitro, amino, C₁₋₃ alkylamino, and di(C₁₋₃)alkylamino, methyl, ethyl, fluoro, chloro, trifluoromethyl, cyano, nitro, and trifluoromethoxy.

In other exemplary embodiments, bi-substituted (aryl- and aza-substituted) compounds for use within the methods and compositions of the invention for treating a neuropathic disorder and/or related symptom(s) are characterized, at least in part, by formula IV, below, which describes in an exemplary manner a methyl substitution on the aryl ring at the same position as found in bicifadine. 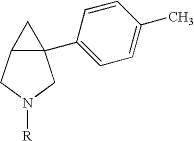
wherein R is selected from, for example, C₁₋₆ alkyl, halo(C₁₋₆)alkyl, C₃₋₉ cycloalkyl, C₁₋₅ alkoxy(C₁₋₆)alkyl, carboxy(C₁₋₃)alkyl, C₁₋₃ alkanoyl, carbamate, halo(C₁₋₃)alkoxy(C₁₋₆)alkyl, C₁₋₃ alkylamino(C₁₋₆)alkyl, and di(C₁₋₃)alkylamino(C₁₋₆)alkyl, cyano(C₁₋₆)alkyl, methyl, ethyl, trifluoromethyl, trifluoroethyl and 2-methoxyethyl.

An illustrative assemblage of bi-substituted 1-aryl-3-azabicyclo[3.1.0]hexanes for use within the methods and compositions of the invention for treating a neuropathic disorder and/or related symptom(s) is provided in Table 6, below. In each of these exemplary compounds, the hydrogen associated with the nitrogen at the ‘3’ position has been replaced with a different aza substituent as shown.

TABLE 6
Exemplary Aza-Substituted 1-aryl-3-azabicyclo [3.1.0] hexanes

| | |
|---|---|
| 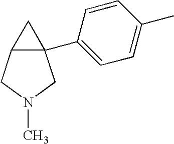 | 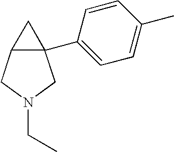 |
| 3-methyl-1-p-tolyl-3-aza- | 3-ethyl-1-p-tolyl-3-aza- |
| bicyclo[3.1.0]hexane | bicyclo[3.1.0]hexane |
| 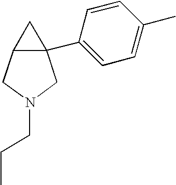 | 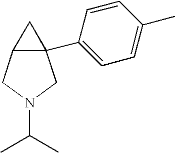 |
| 3-propyl-1-p-tolyl-3-aza- | 3-isopropyl-1-p-tolyl-3-aza- |
| bicyclo[3.1.0]hexane | bicyclo[3.1.0]hexane |
| 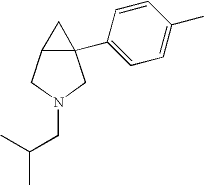 | 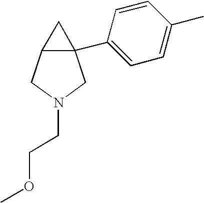 |
| 3-isobutyl-1-p-tolyl-3-aza- | 3-(2-methoxyethyl)-1-p-tolyl- |
| bicyclo[3.1.0]hexane | 3-aza-bicyclo[3.1.0]hexane |
| 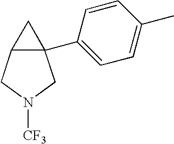 | 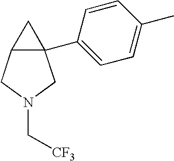 |
| 1-p-tolyl-3-(trifluoromethyl)- | 1-p-tolyl-3-(2,2,2-trifluoroethyl)- |
| 3-aza-bicyclo[3.1.0]hexane | 3-aza-bicyclo[3.1.0]hexane |

The aryl-substituted and aza-substituted 1-aryl-3-azabicyclo[3.1.0]hexanes for use within the methods and compositions of the invention for treating a neuropathic disorder and/or related symptom(s) are useful in any of a variety of forms, including pharmaceutically acceptable, active salts, solvates, hydrates, polymorphs, and/or prodrugs of the compounds disclosed herein, or any combination thereof.

In additional detailed embodiments, the methods and compositions of the invention for treating a neuropathic disorder and/or related symptom(s) employ a 1-aryl-3-azabicyclo[3.1.0]hexane having two or more substituents on the aryl ring. In more detailed aspects, these multiply aryl-substituted compounds for use within the method and compositions of the invention for treating neuropathic disorders and/or related symptoms are characterized, at least in part, by formula V, below:

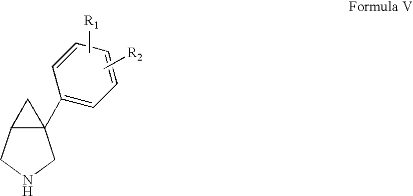

wherein R₁ and R₂ are, independently, for example, halogen, C₁₋₃ alkyl, C₂₋₄ alkenyl, C₂₋₄ alkynyl, halo(C₁₋₃)alkyl, cyano, hydroxy, C₃₋₅ cycloalkyl, C₁₋₃ alkoxy, C₁₋₃ alkoxy(C₁₋₃)alkyl, carboxy(C₁₋₃)alkyl, C₁₋₃ alkanoyl, halo(C₁₋₃)alkoxy, nitro, amino, C₁₋₃ alkylamino, and di(C₁₋₃)alkylamino, methyl, ethyl, fluoro, chloro, trifluoromethyl, cyano, and trifluoromethoxy.

In one exemplary embodiment, the methods and compositions of the invention for treating a neuropathic disorder and/or related symptom(s) employ a multiply aryl-substituted azabicyclo[3.1.0]hexane comprising a racemic or enantiomeric form of 1-(3,4-dichlorophenyl)-3-azabicyclo[3.1.0]hexane. The racemic form of this compound was described in U.S. Pat. No. 4,435,419, incorporated herein by reference). Additional description relating to this compound, and of its enantiomeric forms, processes for resolving the enantiomeric forms, and proposed therapeutic uses for the compound, is provided in U.S. Pat. No. 4,196,120; U.S. Pat. No. 4,231,935; U.S. Pat. No. 6,204,284; U.S. Pat. No. 6,372,919; U.S. patent application Ser. No. 10/466,457; U.S. patent application Ser. No. 10/920,748; U.S. Pat. No. 6,659,887; U.S. Pat. No. 6,716,868; U.S. patent application Ser. No. 10/764,371; U.S. patent application Ser. No. 10/764,373; U.S. patent application Ser. No. 10/764,375 (each incorporated herein by reference. Both the racemic (±)-1-(3,4-dichlorophenyl)-3-azabicyclo[3.1.0]hexane, and its respective (+) and (−) enantiomeric forms provide useful candidate compounds for use within the methods and compositions of the invention for treating a neuropathic disorder and/or related symptom(s), as exemplified herein below.

As further described herein below, a large assemblage of novel, multiply aryl-substituted candidate compounds are also provided for use within the methods and compositions of the present invention for treating neuropathic disorders and/or related symptoms. These novel, multiply aryl-substituted candidate compounds, which have been made and characterized as illustrative embodiments of the invention, include the following (Table 7):

TABLE 7
Exemplary 1-aryl-3-azabicyclo[3.1.0]hexanes
having multiple substitutions on the aryl ring

| | |
|---|---|
| 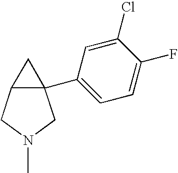 | 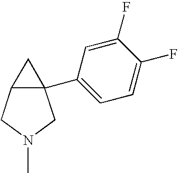 |
| 1-(3-chloro-4-fluorophenyl)-3- | 1-(3,4-difluorophenyl)-3-methyl-3- |
| methyl-3-aza-bicyclo[3.1.0]hexane | aza-bicyclo[3.1.0]hexane |
| 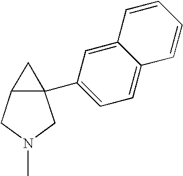 | 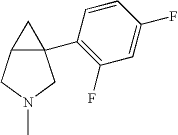 |
| 3-methyl-1-(naphthalen-2-yl)-3- | 1-(2,4-difluorophenyl)-3-methyl-3- |
| aza-bicyclo[3.1.0]hexane | aza-bicyclo[3.1.0]hexane |
| 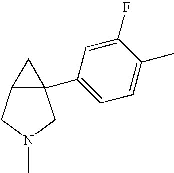 | 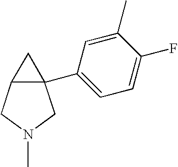 |
| 1-(3-fluoro-4-methylphenyl)-3- | 1-(4-fluoro-3-methylphenyl)-3- |
| methyl-3-aza-bicyclo[3.1.0]hexane | methyl-3-aza-bicyclo[3.1.0]hexane |
| 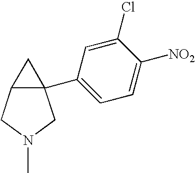 | 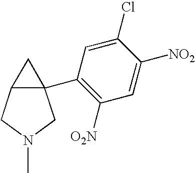 |
| 1-(3-chloro-4-nitrophenyl)-3- | 1-(5-chloro-2,4-dinitrophenyl)-3- |
| methyl-3-aza-bicyclo[3.1.0]hexane | methyl-3-aza-bicyclo[3.1.0]hexane |
| 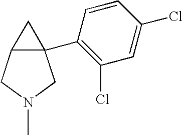 | 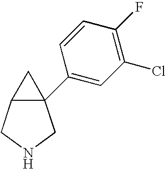 |
| 1-(2,4-dichlorophenyl)-3-methyl- | 1-(3-chloro-4-fluorophenyl)-3- |
| 3-aza-bicyclo[3.1.0]hexane | aza-bicyclo[3.1.0]hexane |

It will be understood that the exemplary, multiply aryl-substituted compounds identified in Table 7 are illustrative, and that the subject modifications comprising multiple aryl substitutions can be varied to comprise other substituents, can include yet additional substituents (i.e., three or more substitutions on the aryl ring), combined with one another, or additionally combined with an “aza substitution” as described herein, to yield yet additional candidate compounds for use within the methods and compositions of the invention for treating a neuropathic disorder and/or related symptom(s). For example, certain embodiments of the invention employ a compound from an illustrative assemblage of 1-(3,4-dichlorophenyl)-3-azabicyclo[3.1.0]hexanes having multiple substitutions, (e.g., as illustrated by multiple chloro substitutions) on the aryl ring, combined with an “aza substitution” on the nitrogen at the ‘3’ position. These, aza-substituted, 1-(3,4-dichlorophenyl)-3-azabicyclo[3.1.0]hexanes useful as candidate compounds within the methods and compositions of the invention for treating a neuropathic disorder and/or related symptom(s) include the following, exemplary compounds, which have been made and characterized as illustrative embodiments (Table 8). The subject compounds are depicted as hydrochloride salts, whereas it will be understood that the invention encompass all forms of the compounds as described herein, including free base forms, and all pharmaceutically acceptable salts, polyrmorphs, solvates, hydrates, and prodrugs:

TABLE 8
Exemplary 1-aryl-3-azabicyclo[3.1.0]hexanes having multiple
substitutions on the aryl ring combined with an aza substitution

| | |
|---|---|
| 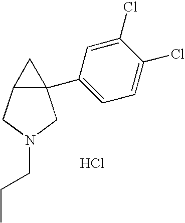 | 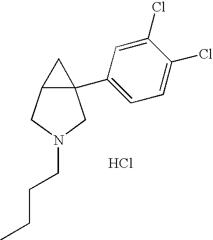 |
| 1-(3,4-dichlorophenyl)-3-propyl-3- | 1-(3,4-dichlorophenyl)-3-butyl-3- |
| azabicyclo[3.1.0]hexane | azabicyclo[3.1.0]hexane |
| hydrochloride | hydrochloride |
| 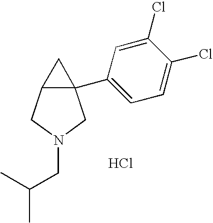 | 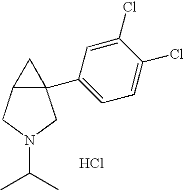 |
| 1-(3,4-dichlorophenyl)-3-isobutyl- | 1-(3,4-dichlorophenyl)-3-isopropyl- |
| 3-azabicyclo[3.1.0]hexane | 3-azabicyclo[3.1.0]hexane |
| hydrochloride | hydrochloride |
| 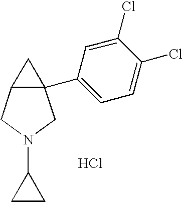 | 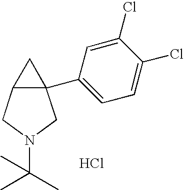 |
| 1-(3,4-dichlorophenyl)-3- | 3-tert-butyl-1-(3,4-dichlorophenyl)-3- |
| cyclopropyl-3-azabicyclo[3.1.0] | aza-bicyclo[3.1.0]hexane |
| hexane hydrochloride | hydrochloride |

Within related aspects of the invention, enantiomeric forms of 1-aryl-3-azabicyclo[3.1.0]hexanes having single or multiple substitutions on the aryl ring, optionally combined with an aza substitution, as described above, are employed within the methods and compositions of the invention for treating a neuropathic disorder and/or related symptom(s). In certain embodiments, the methods and compositions of the invention employ enantiomers, diastereomers, and other stereoisomeric forms of the disclosed compounds, including racemic and resolved forms and mixtures thereof. The present invention encompasses all such forms, including all racemic and resolved isomeric forms, and mixtures thereof. Enantiomeric forms of active compounds within the methods and compositions of the invention can be resolved and isolated according to methods that are well known to those of ordinary skill in the art, including, but not limited to, formation of diastereoisomeric salts or complexes which may be separated by methods including, but not limited to: crystallization; gas-liquid or liquid chromatography; selective reaction of one enantiomer with an enantiomer-specific reagent, for example enzymatic oxidation or reduction, followed by separation of the modified and unmodified enantiomers; and gas-liquid or liquid chromatography in a chiral environment, for example on a chiral support, for example silica with a bound chiral ligand or in the presence of a chiral solvent. Alternatively, specific enantiomers may be synthesized by asymmetric synthesis using optically active reagents, substrates, catalysts or solvents, or by converting one enantiomer to the other by asymmetric transformation. When the compounds described herein contain olefinic double bonds or other centers of geometric asymmetry, and unless specified otherwise, it is intended to include both E and Z geometric isomers. All tautomers are intended to be encompassed by the present invention as well. Exemplary enantiomers of 1-aryl-3-azabicyclo[3.1.0]hexanes having single or multiple substitutions on the aryl ring for use within the invention, which have been resolved and characterized as illustrative embodiments, include the following (Table 9):

TABLE 9
Exemplary enantiomeric compounds

| | |
|---|---|
| 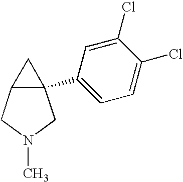 | 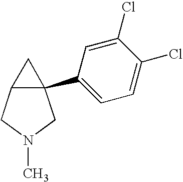 |
| (1R)-1-(3,4-dichlorophenyl)-3- | (1S)-1-(3,4-dichlorophenyl)-3- |
| methyl-3-aza-bicyclo[3.1.0]hexane | methyl-3-aza-bicyclo[3.1.0]hexane |
| 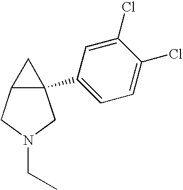 | 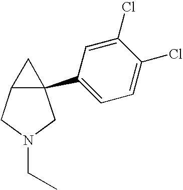 |
| (1R)-1-(3,4-dichlorophenyl)-3- | (1S)-1-(3,4-dichlorophenyl)-3-ethyl- |
| ethyl-3-aza-bicyclo[3.1.0]hexane | 3-aza-bicyclo[3.1.0]hexane |
| 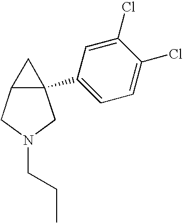 | 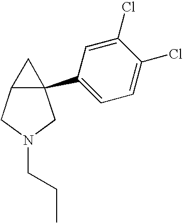 |
| (1R)-1-(3,4-dichlorophenyl)-3- | (1S)-1-(3,4-dichlorophenyl)-3- |
| propyl-3-aza-bicyclo[3.1.0]hexane | propyl-3-aza-bicyclo[3.1.0]hexane |
| 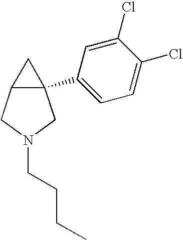 | 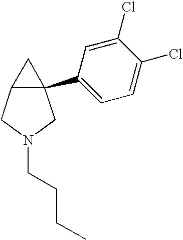 |
| (1R)-3-butyl-1-(3,4- | (1S)-3-butyl-1-(3,4-dichlorophenyl)- |
| dichlorophenyl)-3-aza- | 3-aza-bicyclo[3.1.0]hexane |
| bicyclo[3.1.0]hexane | |
| 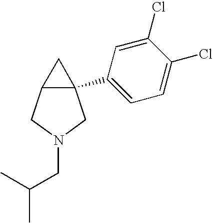 | 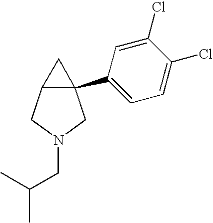 |
| (1R)-1-(3,4-dichlorophenyl)-3- | (1S)-1-(3,4-dichlorophenyl)-3- |
| isobutyl-3-aza- | isobutyl-3-aza- |
| bicyclo[3.1.0]hexane | bicyclo[3.1.0]hexane |
| 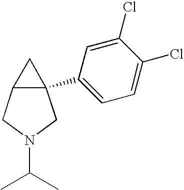 | 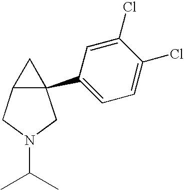 |
| (1R)-1-(3,4-dichlorophenyl)-3- | (1S)-1-(3,4-dichlorophenyl)-3- |
| isopropyl-3-aza- | isopropyl-3-aza- |
| bicyclo[3.1.0]hexane | bicyclo[3.1.0]hexane |
| 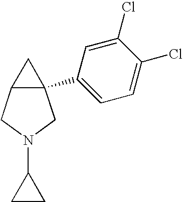 | 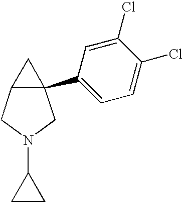 |
| (1R)-3-cyclopropyl-1-(3,4- | (1S)-3-cyclopropyl-1-(3,4- |
| dichlorophenyl)-3- | dichlorophenyl)-3- |
| aza-bicyclo[3.1.0]hexane | aza-bicyclo[3.1.0]hexane |
| 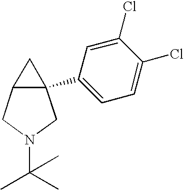 | 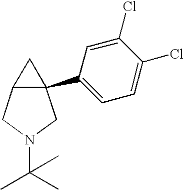 |
| (1R)-3-tert-butyl-1-(3,4- | (1S)-3-tert-butyl-1-(3,4- |
| dichlorophenyl)-3- | dichlorophenyl)-3- |
| aza-bicyclo[3.1.0]hexane | aza-bicyclo[3.1.0]hexane |

As noted above, in certain embodiments the methods and compositions of the invention for treating a neuropathic disorder and/or related symptom(s) employ pharmaceutically acceptable acid addition and base salts of the above-described compounds. Suitable acid addition salts are formed from acids, which form non-toxic salts and examples are hydrochloride, hydrobromide, hydroiodide, sulphate, hydrogen sulphate, nitrate, phosphate, and hydrogen phosphate. Examples of pharmaceutically acceptable addition salts include inorganic and organic acid addition salts. The pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; organic acid salts such as acetate, citrate, lactate, succinate, tartrate, maleate, fumarate, mandelate, acetate, dichloroacetate, trifluoroacetate, oxalate, formate and the like; sulfonates such as methanesulfonate, benzenesulfonate, p-toluenesulfonate and the like; and amino acid salts such as arginate, asparginate, glutamate, tartrate, gluconate and the like. Suitable base salts are formed from bases, which form non-toxic salts and examples are the aluminum, calcium, lithium, magnesium, potassium, sodium, zinc and diethanolamine salts.

In other detailed embodiments, the methods and compositions of the invention for treating a neuropathic disorder and/or related symptom(s) employ prodrugs of the above-disclosed compounds. Prodrugs are considered to be any covalently bonded carriers which release the active parent drug in vivo. Examples of prodrugs useful within the invention include esters or amides with hydroxyalkyl or aminoalkyl as a substituent, and these may be prepared by reacting such compounds as described above with anhydrides such as succinic anhydride.

The invention disclosed herein will also be understood to encompass methods and compositions for treating a neuropathic disorder and/or related symptom(s) using in vivo metabolic products of the above-described compounds (either generated in vivo after administration of the subject precursor compound, or directly administered in the form of the metabolic product itself). Such products may result for example from the oxidation, reduction, hydrolysis, amidation, esterification and the like of the administered compound, primarily due to enzymatic processes. Accordingly, the invention includes methods and compositions of the invention for treating a neuropathic disorder and/or related symptom(s) employing compounds produced by a process comprising contacting a compound as described above with a mammalin subject for a period of time sufficient to yield a metabolic product thereof. Such products typically are identified by preparing a radiolabelled compound of the invention, administering it parenterally in a detectable dose to an animal such as rat, mouse, guinea pig, monkey, or to man, allowing sufficient time for metabolism to occur and isolating its conversion products from the urine, blood or other biological samples.

The invention disclosed herein will also be understood to encompass the methods and compositions of the invention for treating a neuropathic disorder and/or related symptom(s) employing the above-described compounds isotopically-labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively,

1-Aryl-3-azabicyclo[3.1.0]hexanes for use within the compositions and methods of invention for treating neuropathic disorders and/or related symptoms include the aryl- and/or aza-substituted, bi-substituted, and multiply aryl-substituted 1-aryl-3-azabicyclo[3.1.0]hexanes described herein, as well as, without limitation, all “anti-neuropathically active” 1-aryl-3-azabicyclo[3.1.0]hexanes (i.e., all such compounds that are effective following administration to a mammalian subject in an effective amount, to treat or prevent a neuropathic disorder, or one or more symptom(s) associated with a neuropathic disorder, in the subject), as well as all active, pharmaceutically acceptable salts, polymorphs, enantiomers, solvates, hydrates and/or prodrugs of these compounds, and all combinations of the foregoing compounds or distinct chemical forms thereof as noted above. As used herein, prodrugs includes any 1-aryl-3-azabicyclo[3.1.0]hexane as described herein covalently bonded with a second compound or chemical moiety as a “carrier”, wherein the carrier release the active 1-aryl-3-azabicyclo[3.1.0]hexane in vivo. Examples of prodrugs include esters or amides of any of the compounds described herein, including of compounds depicted in any of Formulae I-V, for example using hydroxyalkyl or aminoalkyl as a substituent, which prodrugs may prepared by reacting a parent 1-aryl-3-azabicyclo[3.1.0]hexane with anhydrides such as succinic anhydride.

The 1-aryl-3-azabicyclo[3.1.0]hexanes for use within the invention will also be understood to include in vivo metabolic products of the above-described compounds. Such products may result for example from the oxidation, reduction, hydrolysis, amidation, esterification and the like of the administered compound, primarily due to enzymatic processes. Accordingly, the invention includes methods and formulations comprising metabolically-processed compounds produced by exposing a 1-aryl-3-azabicyclo[3.1.0]hexane as described herein to a physiological compartment within a mammal for a period of time sufficient to yield a metabolic product of the 1-aryl-3-azabicyclo[3.1.0]hexane. Such products can be readily identified by preparing a radiolabelled 1-aryl-3-azabicyclo[3.1.0]hexane, administering it to a mammalian subject (e.g., parenterally, allowing sufficient time for metabolism to occur, and isolating the metabolic conversion products of the administered compound from the urine, blood or other biological samples of the subject.

The instant invention will also be understood to encompass related methods and compositions wherein the subject 1-aryl-3-azabicyclo[3.1.0]hexanes are labeled with a detectable label moiety for various known clinical and diagnostic uses. For example, the 1-aryl-3-azabicyclo[3.1.0]hexanes may be isotopically-labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine and chlorine, such as 2H, 3H, 13C, 14C, 15N, 18O, 17O, 31P, 32P, 35S, 18F, and 36Cl, respectively. Other useful labeling moieties in this context may include any detectable chemical moiety, for example conventional fluorophores, chemiluminescers, and enzymes (e.g., alkaline phosphatase, peroxidase, and P-galactosidase). Enzyme labels are readily detectable by addition of a corresponding chromogenic substrate and detecting the resulting color or fluorescent signal.

As noted above, the 1-aryl-3-azabicyclo[3.1.0]hexanes for use within methods and compositions of the invention for treating or preventing neuropathic disorders and/or related symptoms will be useful in active, pharmaceutically acceptable acid addition and base salts thereof. Suitable acid addition salts are formed from acids, which form non-toxic salts, exemplified by hydrochloride, hydrobromide, hydroiodide, sulphate, hydrogen sulphate, nitrate, phosphate, and hydrogen phosphate salts. Examples of pharmaceutically acceptable addition salts include inorganic and organic acid addition salts, including but not limited to: metal salts such as sodium salts, potassium salts, cesium salts and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; organic acid salts such as acetate, citrate, lactate, succinate, tartrate, maleate, fumarate, mandelate, acetate, dichloroacetate, trifluoroacetate, oxalate, formate and the like; sulfonates such as methanesulfonate, benzenesulfonate, p-toluenesulfonate and the like; and amino acid salts such as arginate, asparginate, glutamate, tartrate, gluconate and the like. Suitable base salts are formed from bases, which form non-toxic salts and examples are the aluminum, calcium, lithium, magnesium, potassium, sodium, zinc and diethanolamine salts.

The various 1-aryl-3-azabicyclo[3.1.0]hexanes for use within the the methods and compositions of the invention for treating a neuropathic disorder and/or related symptom(s) can be produced according to a variety of known synthetic methods, as well as by additional, previously undisclosed methods, as described herein below.

Available methods for synthesizing aryl substituted 3-azabicyclo[3.1.0]hexanes are limited. Bicifadine hydrochloride has been previously produced as described in U.S. Pat. No. 4,131,611, U.S. Pat. No. 4,196,120, U.S. Pat. No. 4,231,935, and in Epstein et al., J. Med. Chem. 24:481, 1981. An exemplary prior synthetic method for producing bicifadine hydrochloride is outlined in Scheme A, below.

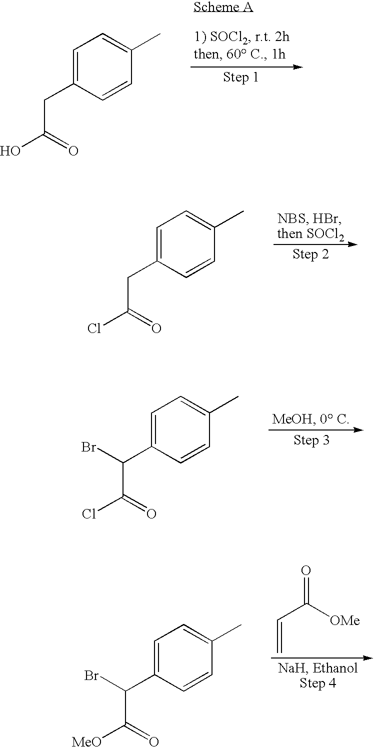

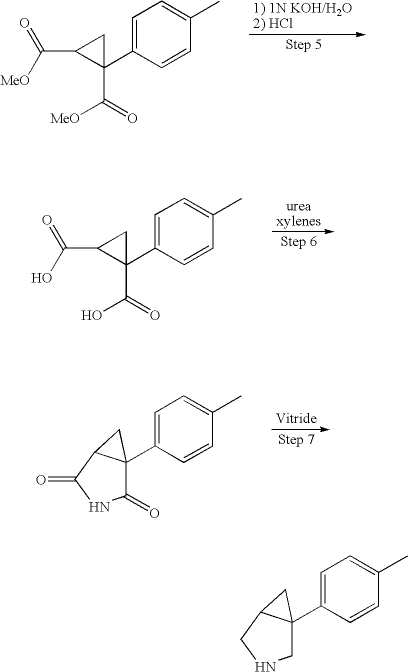

This synthetic scheme starts with preparation of the 2-bromo-2-(p-tolyl)-acetate in 3 steps. The dimethyl-1-(4-methylphenyl)-1,3-cyclopropanedicarboxylate is prepared from the bromoester reacting with methyl acrylate. The diester is converted into the diacid, which is condensed with urea to produce 1-(p-tolyl)-1,2-cyclopropanedicarboximde. Then, the 1-(p-tolyl)-1-cyclopropanedicarboximde is reduced to an amine by Vitride and converted to the hydrochloride salt to yield the bicifadine hydrochloride.

U.S. Pat. No. 4,118,417 discloses a process for resolving a (+)-1-(p-methylphenyl)-1,2-cyclopropanedicarboxylic acid with S-(−)-1-(1-naphthyl)ethylamine, and its conversion to (+)-bicifadine, as illustrated below in synthetic Scheme B. The (−)-bicifadine is also reported to be producible from the corresponding (−)-1-(p-methylphenyl)-1,2-cyclopropanedicarboxylic acid.

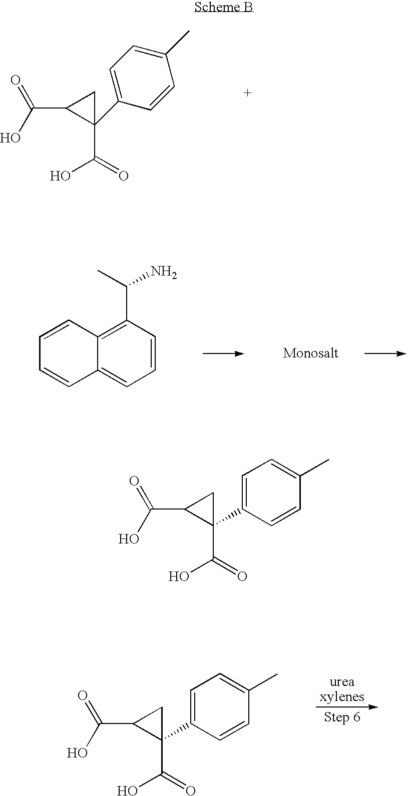

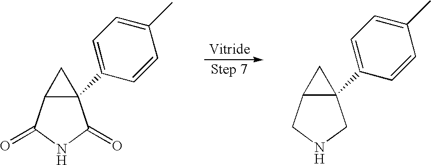

Additional methods and compositions to produce bicifadine and other substituted 1-aryl-3-azabicyclo[3.1.0]hexanes. Reaction Scheme 1 below generally sets forth an exemplary process for preparing bicifadine from a known methyl 2-bromo-2-p-tolylacetate or methyl 2-chloro-2-p-tolylacetate. The bromo or chloro acetate react with acrylonitrile to provide the methyl 2-cyano-1-p-tolylcyclopropanecarboxylate, which is then reduced into the amino alcohol by reducing agents such as lithium aluminum hydride (LAH) or sodium aluminum hydride (SAH) or NaBH₄ with ZnCl₂. Cyclization of the amino alcohol with SOCl₂ or POCl₃ will provide the 1-(4-methylphenyl)-3-azabicyclo[3.1.0]hexane. The cyclization of substituted 4-aminobutan-1-ol by SOCl₂ or POCl₃ into the pyrrolidine ring system was reported by Armarego et al., J. Chem. Soc. [Section C: Organic] 19:3222-9, 1971, and in patent publication PL 120095 B2, CAN 99:158251 by Szalacke et al. Oxalyl chloride, phosphorous tribromide, triphenylphosphorous dibromide, oxalyl bromide may be used for the same purpose. The methyl 2-bromo-2-p-tolylacetate or methyl 2-chloro-2-p-tolylacetate may be synthesized from p-methyl benzoylaldehyde or methyl-2-p-tolylacetate as shown in Reaction Scheme 1A.

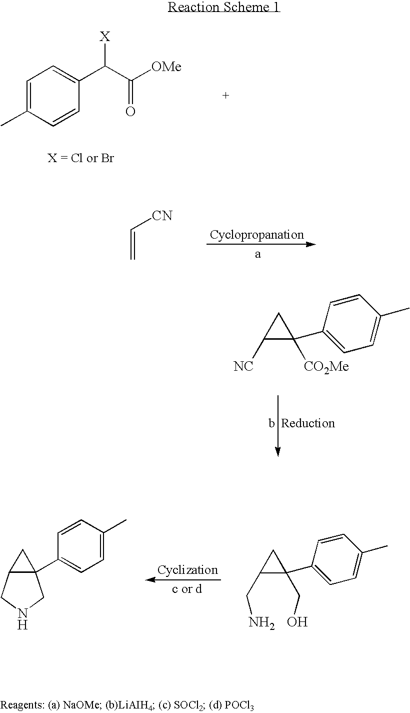

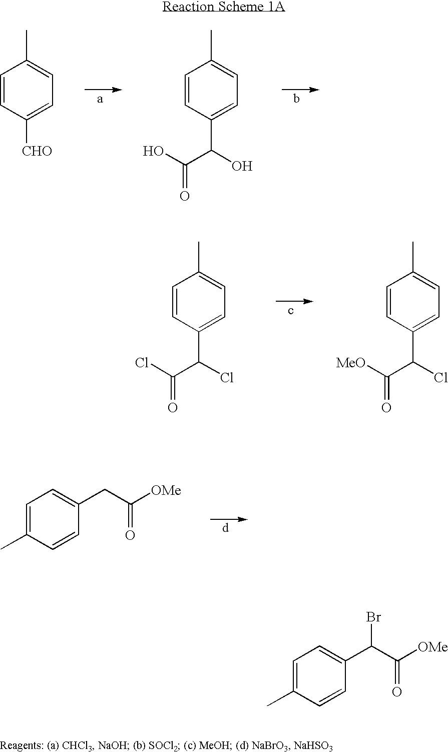

Reaction Scheme 2 below illustrates another exemplary process for transforming the methyl 2-cyano-1-p-tolylcyclopropanecarboxylate to a desired compound or intermediate of the invention. Hydrolysis of the cyano ester provides the potassium salt which can then be converted into the cyano acid. Reduction and cyclization of the 2-cyano-1-p-tolylcyclopropanecarboxylic acid with LAH or LiAlH(OMe)₃ according to the procedure outlined in Vilsmaier et al., Tetrahedron 45:3683-3694, 1989, will generate bicifadine. In addition, the cyano-1-p-tolylcyclopropanecarboxylic acid can be hydrogenated and cyclized into an amide, which is then reduced into bicifadine.

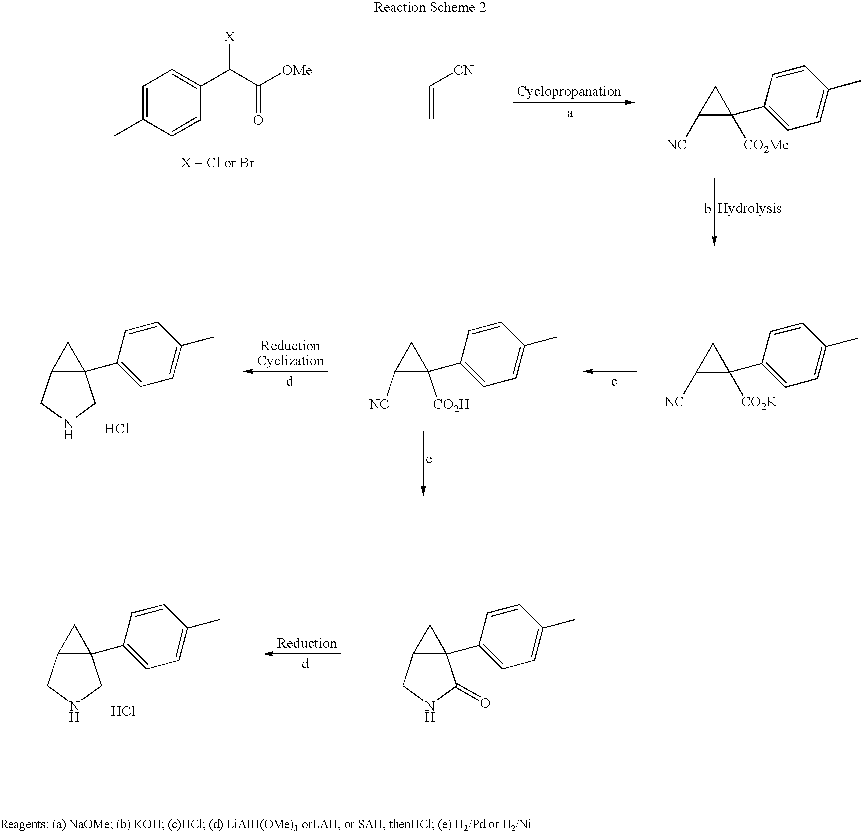

Reaction Scheme 3 below discloses an alternative exemplary process for converting the methyl 2-cyano-1-p-tolylcyclopropanecarboxylate to a desired compound or intermediate of the invention. The methyl 2-cyano-1-p-tolylcyclopropanecarboxylate is reduced and cyclized into 1-p-tolyl-3-aza-bicyclo[3.1.0]hexan-2-one, which is then reduced to bicifadine (Marazzo et al., Arkivoc v: 156-169, 2004).

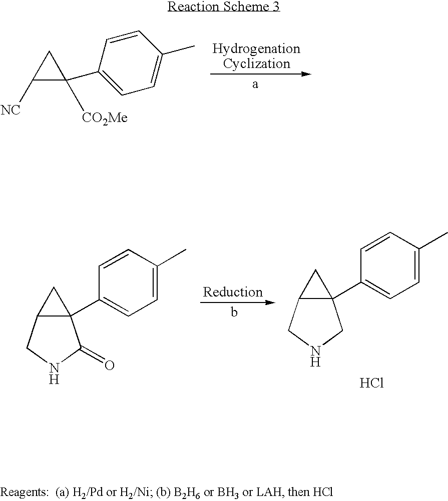

Reaction Scheme 4 below provides another exemplary process to prepare bicifadine. Reaction of 2-p-tolylacetonitrile with (±)-epichlorohydrin gives approximately a 65% yield of 2-(hydroxymethyl)-1-p-tolylcyclopropanecarbonitrile (85% cis) with the trans isomer as one of the by-products (Cabadio et al., Fr. Bollettino Chimico Farmaceutico 117:331-42, 1978; Mouzin et al., Synthesis 4:304-305, 1978). The methyl 2-cyano-1-p-tolylcyclopropanecarboxylate can then be reduced into the amino alcohol by a reducing agent such as LAH, SAH or NaBH₄ with ZnCl₂ or by catalytic hydrogenation. Cyclization of the amino alcohol with SOCl₂ or POCl₃ provides the 1-(4-methylphenyl)-3-azabicyclo[3.1.0]hexane. The cyclization of substituted 4-aminobutan-1-ol by SOCl₂ or POCl₃ into the pyrrolidine ring system has been reported previously (Armarego et al., J. Chem. Soc. [Section C: Organic] 19:3222-9, 1971; and patent publication PL 120095 B2, CAN 99:158251).

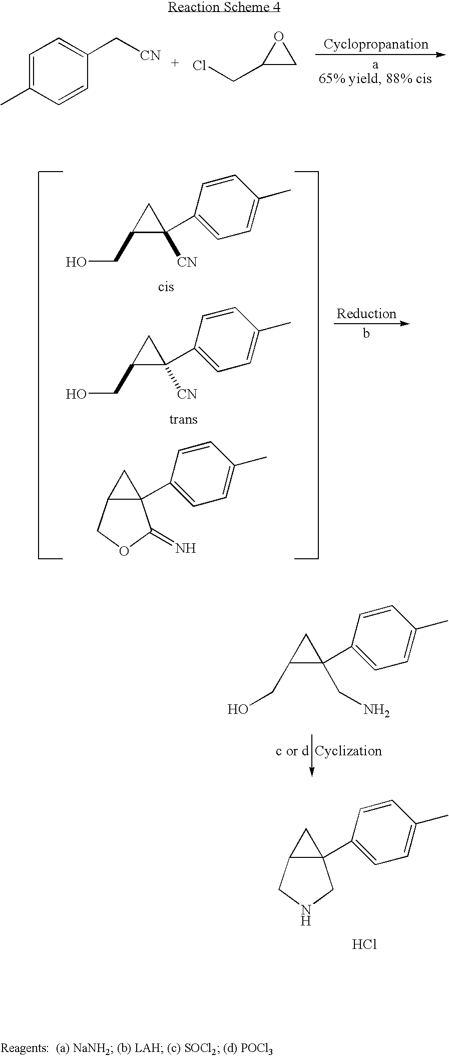

Reaction Scheme 5 provides an exemplary process for synthesizing the (1R,5S)-(+)-1-(4-methylphenyl)-3-azabicyclo[3.1.0]hexane hydrochloride or (+)-bicifadine. Using (S)-(+)-Epichlorohydrin as a starting material in the same process described in Scheme 4 will ensure that the final product with IR chirality (Cabadio et al., Fr. Bollettino Chimico Farmaceutico 117:331-42, 1978).

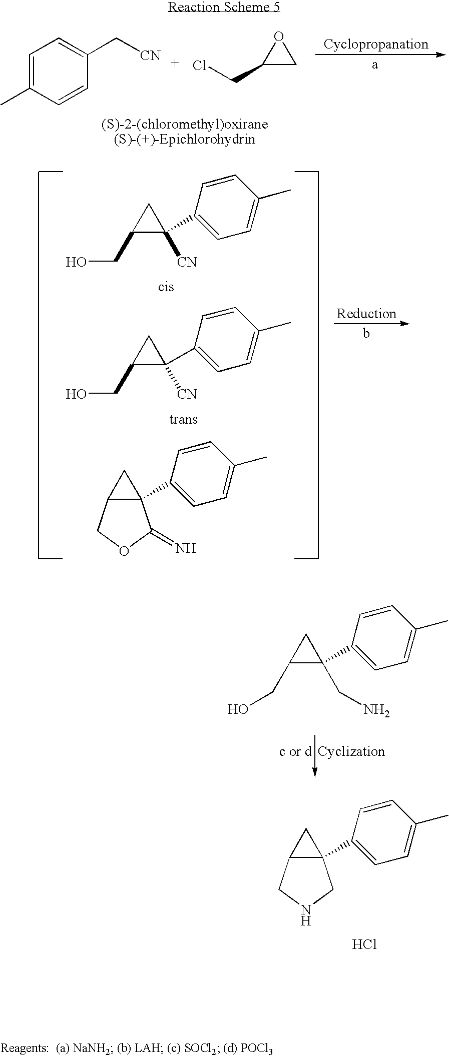

Reaction Scheme 6 provides an exemplary process to prepare the (1S,5R)-(−)-1-(4-methylphenyl)-3-azabicyclo[3.1.0]hexane hydrochloride or the (−)-bicifadine. Using (R)-(−)-Epichlorohydrin as a starting material in the same process described in Scheme 4 will ensure a final product with 1S chirality (Cabadio et al., Fr. Bollettino Chimico Farmaceutico 117:331-42, 1978).

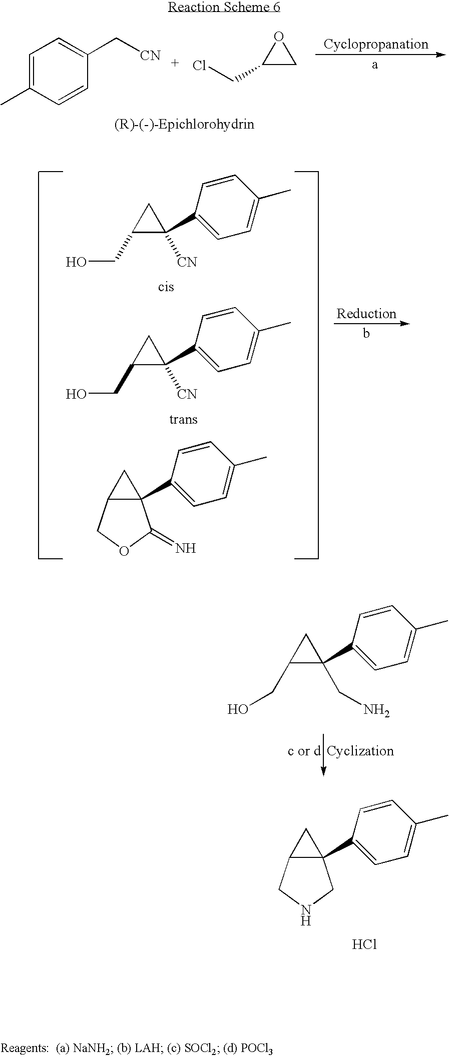

Reaction Scheme 7 provides an alternative exemplary process for transforming the 2-(hydroxymethyl)-1-p-tolylcyclopropanecarbonitrile to a desired compound or intermediate of the invention via an oxidation and cyclization reaction. Utilizing chiral starting materials (+)-epichlorohydrin or (−)-epichlorohydrin will lead to the corresponding (+)- or (−)-bicifadine through the same reaction sequences.

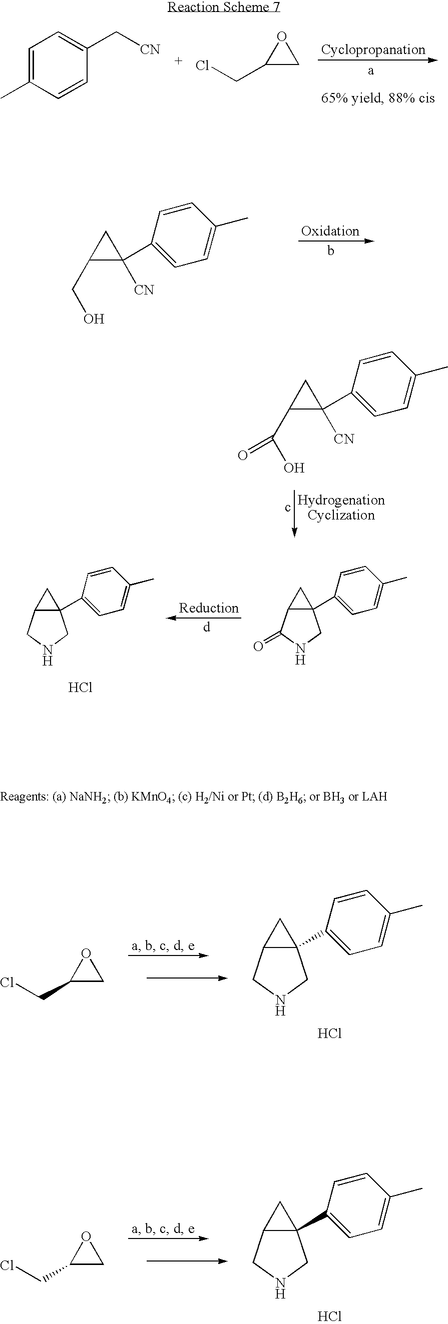

Reaction Scheme 8 provides an exemplary process for transforming the epichlorohydrin to a desired compound or intermediate of the invention via a replacement and cyclization reaction. The reaction of methyl 2-p-tolylacetate with epichlorohydrin gives methyl 2-(hydroxymethyl)-1-p-tolylcyclopropanecarboxylate with the desired cis isomer as the major product. The alcohol is converted into an OR₃ group such as —O-mesylate, —O-tosylate, —O-nosylate, —O-brosylate, —O-trifluoromethanesulfonate. Then OR₃ is replaced by a primary amine NH₂R₄, where R₄ is a nitrogen protection group such as a 3,4-dimethoxy-benzyl group or other known protection group. Nitrogen protecting groups are well known to those skilled in the art, see for example, “Nitrogen Protecting Groups in Organic Synthesis”, John Wiley and sons, New York, N.Y., 1981, Chapter 7; “Nitrogen Protecting Groups in Organic Chemistry”, Plenum Press, New York, N.Y., 1973, Chapter 2; See also, T. W. Green and P. G. M. Wuts in “Protective Groups in Organic Chemistry, 3rd edition” John Wiley & Sons, Inc. New York, N.Y., 1999. When the nitrogen protecting group is no longer needed, it may be removed by methods well known in the art. This replacement reaction is followed by a cyclization reaction which provides the amide, which is then reduced into amine by a reducing agent such as LAH. Finally the protection group is removed to yield the bicifadine. Utilizing chiral (S)-(+)-Epichlorohydrin as a starting material leads to the (1R,5S)-(+)-1-(4-methylphenyl)-3-azabicyclo[3.1.0]hexane hydrochloride or (+)-bicifadine with the same reaction sequence. Similarly, the (R)-(−)-Epichlorohydrin will lead to the (1S,5R)-(−)-1-(4-methylphenyl)-3-azabicyclo[3.1.0]hexane hydrochloride or the (−)-bicifadine.

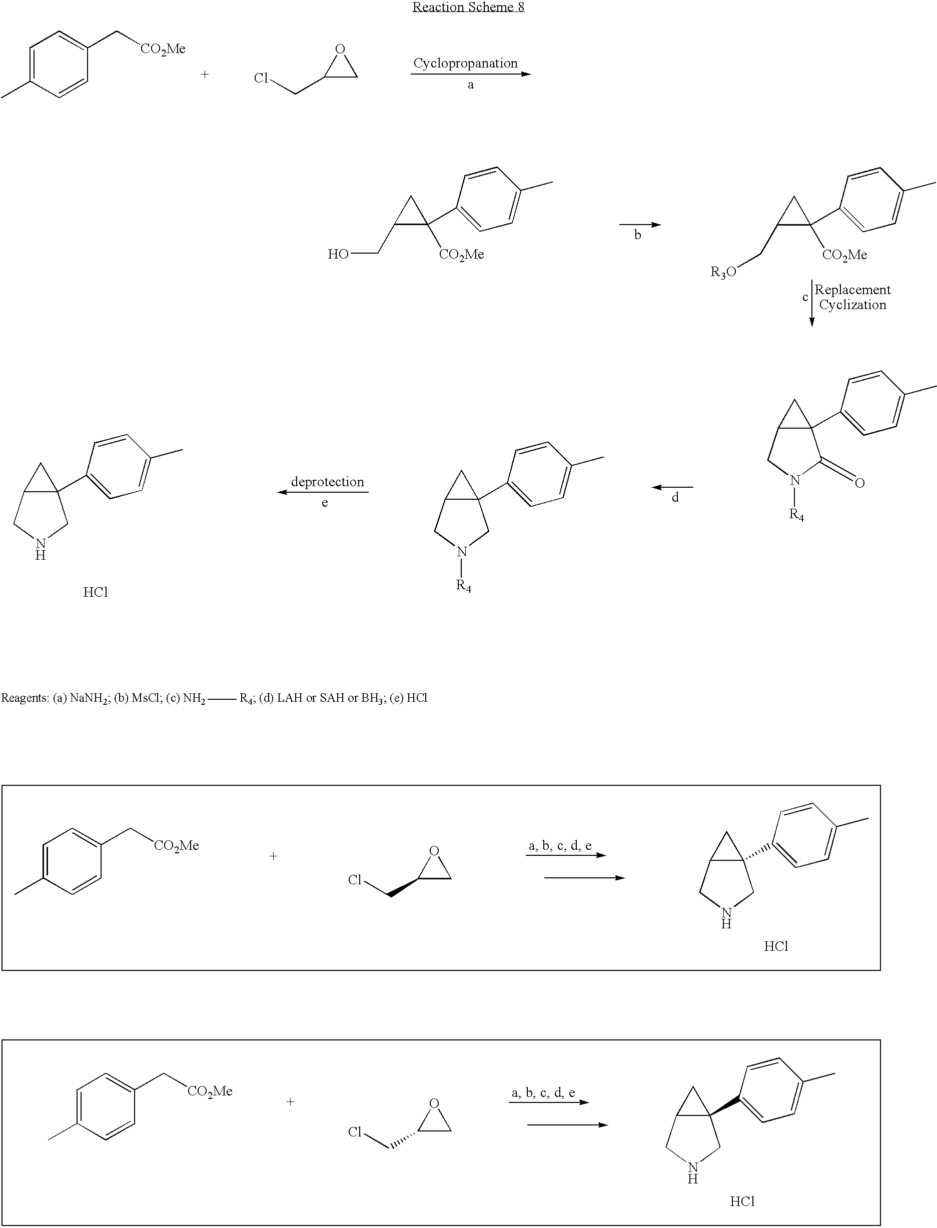

Reaction Scheme 9 provides an exemplary process for transforming the diol to a desired compound or intermediate of the invention. Reduction of the diester provides the diol which is then converted into an OR₃ group such as —O-mesylate, —O-tosylate, —O-nosylate, —O-brosylate, —O-trifluoromethanesulfonate. Then OR₃ is replaced by a primary amine NH₂R₆, where R₆ is a nitrogen protection group such as a 3,4-dimethoxy-benzyl group or other protection groups known in the art (e.g., allyl amine, tert-butyl amine). When the nitrogen protecting group is no longer needed, it may be removed by methods known to those skilled in the art.

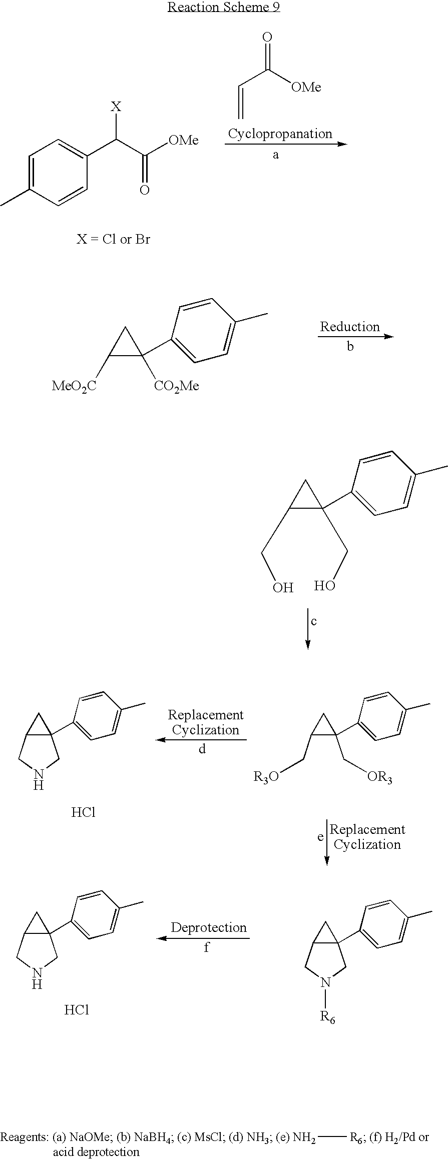

Reaction Scheme 10 provides an exemplary process for resolving the 1-p-tolyl-3-aza-bicyclo[3.1.0]hexane to (+)-and (−)-bicifadine. The resolution of amines through tartaric salts is generally known to those skilled in the art. For example, using O,O-Dibenzoyl-2R,3R-Tartaric Acid (made by acylating L(+)-tartaric acid with benzoyl chloride) in dichloroethane/methanol/water, racemic methamphetamine can be resolved in 80-95% yield, with an optical purity of 85-98% (Synthetic Communications 29:4315-4319, 1999).

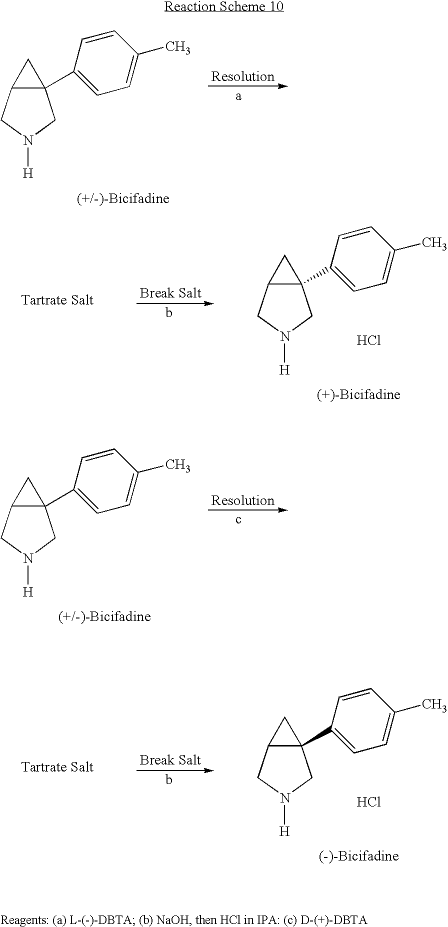

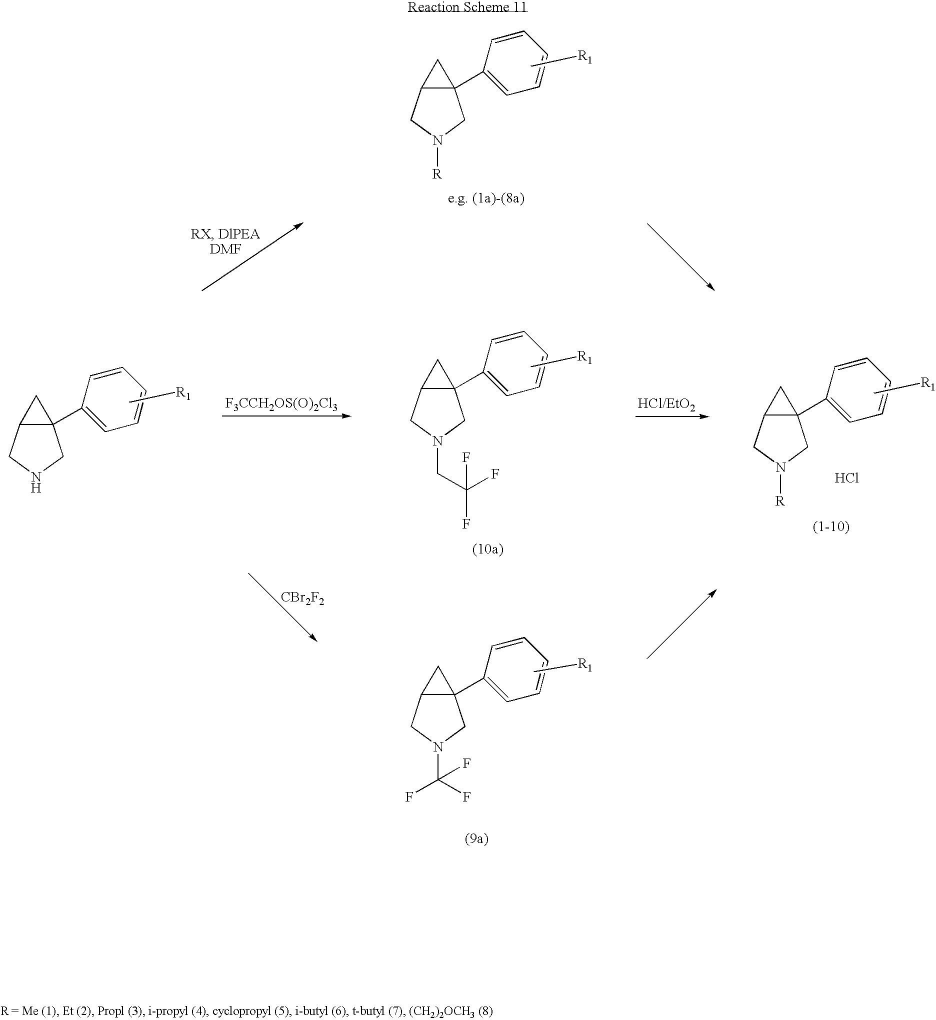

Enantiomers of compounds within the present invention can be prepared as shown in Scheme 12:

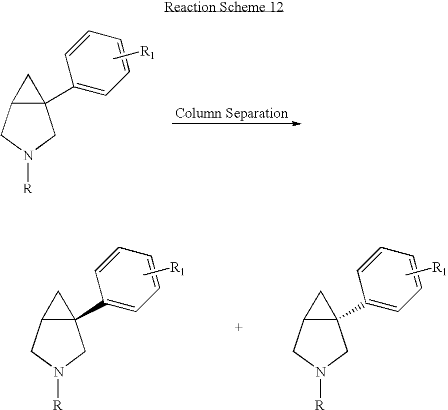

Alternatively, enantiomers of the compounds of the present invention can be prepared as shown in Scheme 13 using alkylation reaction conditions examplied in scheme 11.

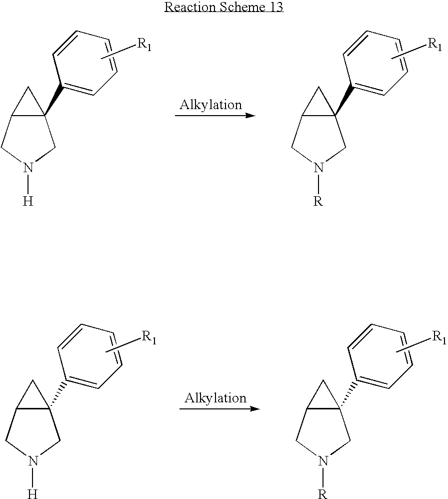

To produce additional subsituted 1-Aryl-3-Azabicyclo[3.1.0]hexanes for use within the the methods and compositions of the invention for treating a neuropathic disorder and/or related symptom(s), the following provides a general procedure for alkylation of 3-azabicyclo[3.1.0]hexanes. To a stirred solution of a 3-azabicyclo[3.1.0]hexane (1 eq) in anhydrous DMF (15 mL) was added diisopropylethylamine (DIPEA) (1.3 eq). The reaction mixture was stirred at room temperature for 20 minutes then alkyl halides (1.3 eq) were added to the reaction mixture and then allowed to stir at room temperature for 2 hours and analysed by TLC. If unreacted starting material remained, the reactions were warmed to 50° C. and held overnight. Reactions were reduced under a high vacuum then dissolved in dichloromethane (20 mL) and washed with water (20 mL). The mixture was passed through a phase separator cartridge. Organics were collected and filtered through a 2 g silica cartridge, fractions were monitored by TLC, the fractions contained the desired product were combined, reduced and analysed by ¹H-NMR. The following compounds are prepared by following the general procedures described above:

Synthesis of 3-Methyl-1-p-tolyl-3-aza-bicyclo[3.1.0]hexane. 0.6871 g (yield: 51%). The compound was analyzed by nuclear magnetic resonance, NMR, confirming the structure produced and the resultant NMR data are listed below.

¹H NMR (300 MHz, δ₆-DMSO) δ 7.10-7.03 (m, 4H, ArH), 3.28 (d, 1H, J=8.5 Hz, NCH₂), 3.07 (d, 1H, J=8.8 Hz, NCH₂), 2.55 (d, 1H, J=8.4 Hz, NCH₂), 2.47 (dd, 1H, J=8.8 Hz, 5.1 Hz, NCH₂), 2.37 (s, 3H, NCH₃), 2.30 (s, 3H, ArCH₃), 1.65 (m, 1H, CH₂CH), 1.38 (t, 1H, J=4.0 Hz, CHCH₂), 0.77 (dd, 1H, J=8.1 Hz, 4.4 Hz, CHCH₂).

Synthesis of 3-Ethyl-1-p-tolyl-3-aza-bicyclo[3.1.0]hexane. 1.0324 g (yield: 72%) ¹H NMR (300 MHz, δ₆-DMSO) δ 7.11-7.04 (m, 4H, ArH), 3.35 (d, 1H, J=8.4 Hz, NCH₂), 3.12 (d, 1H, J=8.5 Hz, NCH₂), 2.56-2.43 (m, 4H, 2×NCH₂, CH₃CH₂), 2.32 (s, 3H, NCH₃), 1.66 (m, 1H, CH₂CH), 1.39 (t, 1H, J=4.4 Hz, CHCH₂), 1.09 (t, 3H, J=7.4 Hz, CH₂CH₃), 0.78 (dd, 1H, J=7.7 Hz, 4.0 Hz, CHCH₂).

Synthesis of 3-Propyl-1-p-tolyl-3-aza-bicyclo[3.1.0]hexane. 0.9284 g (yield: 60%) ¹H NMR (300 MHz, δ₆-DMSO) δ 7.11-7.04 (m, 4H, ArH), 3.34 (d, 1H, J=8.4 Hz, NCH₂),3.12 (d, 1H, J=8.9 Hz, NCH₂), 2.55 (d, 1H, J=8.5 Hz, NCH₂), 2.44 (m, 3H, NCH₂, CH₂CH₂CH₃), 2.32 (s, 3H, ArCH₃), 1.66 (m, 1H, CH₂CH), 1.50 (m, 2H, CH₂CH₂CH₃), 1.39 (t, 1H, J=4.3 Hz, CHCH₂), 0.90 (t, 3H, J=7.4 Hz, CH₂CH₃), 0.77 (dd, 1H, J=7.7 Hz, 4.1 Hz, CHCH₂).

Synthesis of 3-Isopropyl-1-p-tolyl-3-aza-bicyclo[3.1.0]hexane. 0.6645 g (yield: 43%) ¹H NMR (300 MHz, δ₆-DMSO) δ 7.76-7.05 (m, 4H, ArH), 3.38 (d, 1H, J=8.5 Hz, NCH₂), 3.15 (d, 1H, J=8.8 Hz), 2.62 (d, 1H, J=8.4 Hz, NCH₂), 2.52 (dd, 1H, J=8.8 Hz, 3.7 Hz, NCH₂), 2.47 (m, 1H, NCH₂), 2.32 (s, 3H, ArCH₃), 1.66 (m, 1H, CH₂CH), 1.37 (t, 1H, J=4.0 Hz, NCH₂), 1.07 (dd, 6H, J=3.7 Hz, 6.7 Hz, ((CH₃)₂CH), 0.76 (dd, 1H, J=8.1 Hz, 4.1 Hz, CHCH₂).

Synthesis of 3-Isobutyl-1-p-tolyl-3-aza-bicyclo[3.1.0]hexane. 0.8059 g (yield: 49%) ¹H NMR (300 MHz, δ₆-DMSO) δ 7.25-7.05 (m, 4H, ArH), 3.30 (d, 1H, J=8.4 Hz, NCH₂), 3.08 (d, 1H, J=8.5 Hz, NCH₂), 2.51 (d, 1H, J=8.1 Hz, NCH₂), 2.45 (dd, 1H, J=8.4 Hz, 3.6 Hz, NCH₂), 2.34 (s, 3H, ArCH₃), 2.23 (d, 2H, J=7.0 Hz), NCH₂CH), 1.74 (m, 1H, CH₂CH(CH₃)₂), 1.65 (m, 1H, CH₂CH), 1.43 (t, 1H, J=4.1 Hz, CHCH₂), 0.89 (d, 6H, J=6.7 Hz, CH(CH₃)₂), 0.74 (dd, 1H, J=8.1 Hz, 3.7 Hz, CHCH₂).

Synthesis of 3-(2-Methoxyethyl)-1-p-tolyl-3-aza-bicyclo[3.1.0]hexane. 0.092 g (yield: 5%) ¹H NMR (300 MHz, δ₆-DMSO) δ 71.4-7.02 (m, 4H, ArH), 3.46 (t, 3H, J=5.7 Hz, NCH₂CH₂OCH₃), 3.34 (s, 3H, OCH₃), 3.12 (d, 1H, J=8.5 Hz, NCH₂), 2.67 (t, 2H, J=5.9 Hz, NCH₂CH₂)CH₃), 2.60 (d, 1H, J=8.4 Hz, NCH₂), 2.50 (dd, 1H, J=8.8 Hz, 5.1 Hz, NCH₂), 2.31 (s, 3H, ArCH₃), 1.63 (m, 1H, CH₂CH), 1.40 (t, 1H, J=4.1 Hz, CHCH₂), 0.76 (dd, 1H, J=8.0 Hz, 4.4 Hz, CHCH₂).

Synthesis of 1-p-Tolyl-3-trifluoromethyl-3-aza-bicyclo[3.1.0]hexane. To a stirred solution of bicifadine (free base) (1 g, 4.77 mmol) and dibromodifluoromethane (0.87 mL, 9.54 mmol) in DMSO (10 mL) was added tetrakis(dimethylamino)ethylene (2.4 mL, 10.5 mmol) dropwise at room temperature. On complete addition the reaction was stirred at room temperature overnight. The reaction mixture was filtered to remove solid by-products. The filtrate was partitioned between ethyl acetate (50 mL) and saturated sodium bicarbonate solution (50 mL), the organics were collected, dried over magnesium sulphate, filtered and reduced. The crude residue was purified by column chromatography [SiO₂ (30 g): (90 EtOAc: 8 MeOH: 2 NH₄OH)] to give the required material as a yellow oil, 0.6050 g (53%). ¹H NMR (300 MHz, δ₆-DMSO) δ 7.16-7.06 (m, 4H, ArH), 3.97 (t, 1H, J=6.3 Hz, NCH₂), 3.78 (s, 3H, NCH₂), 2.34 (s, 3H, ArCH₃), 1.87 (m, 1H, CHCH₂), 1.19 (t, 1H, J=5.5 Hz, CHCH₂), 0.87 (m, 1H, CHCH₂).

Synthesis of 1-p-Tolyl-3-(2,2,2-trifluoroethyl)-3-aza-bicyclo[3.1.0]hexane. A solution of bicifadine (2 g, 9.54 mmol) and triethylamine (1.33 mL, 9.54 mmol) and 2,2,2-trifluoroethyltrichloromethane sulphonate (0.7 mL, 4.4 mmol) in toluene (20 mL) was heated to reflux and held at this temperature until complete conversion by TLC was observed. Reaction mixture was partitioned between ethyl acetate (50 mL) and saturated sodium bicarbonate solution (50 mL). Organics were isolated, dried over magnesium sulphate, filtered and reduced. Crude material was purified by column chromatography [SiO₂ (30 g): (90 EtOAc: 8 MeOH: 2 NH₄OH)] to give the required material as a yellow oil, 0.9149 g (75%). ¹H NMR (300 MHz, δ₆-DMSO) 7.26-7.05 (m, 4H, ArH), 3.44 (d, 1H, J=8.1 Hz, NCH₂), 3.23-3.08 (m, 3H, CH₂CF₃, NCH₂), 2.90 (d, 1H, J=8.1 Hz, NCH₂), 2.84 (dd, 1H, J=8.1 Hz, 4.1 Hz, NCH₂), 2.37 (s, 3H, ArCH₃), 1.71 (m, 1H, CH₂CH), 1.38 (t, 1H, J=4.4 Hz, CHCH₂), 0.83 (dd, 1H, J=7.7 Hz, 4.0 Hz, CHCH₂).

General Procedure for Hydrochloride Salt Formation. To a stirred solution of free base (1 mol equiv.) in anhydrous diethyl ether (5 mL) was added 1 M HCl in ether (5 mol equiv.) dropwise. On complete addition the reaction mixture was stirred at ice bath temperature for 30 minutes. The resultant solids were isolated by filtration, washing with cold diethyl ether (5 mL). The isolated solids were oven dried and analyzed by ¹H-NMR, ¹³C-NMR and MS.

Synthesis of 3-Methyl-1-p-tolyl-3-aza-bicyclo[3.1.0]hexane hydrochloride. ¹H NMR (300 MHz, δ₆-DMSO) δ 11.36 (s, 1H, NHCl), 7.20-7.12 (m, 4H, ArH), 3.86 (dd, 1H, J=11.0 Hz, 5.1 Hz, NCH₂), 3.60 (dd, 1H, J=11.1 Hz, 5.2 Hz, NCH₂), 3.53-3.43 (m, 2H, 2×NCH₂), 2.80 (s, 3H, NCH₃), 2.28 (s, 3H, ArCH₃), 2.07 (m, 1H, CHCH₂), 1.81 (t, 1H, J=5.2 Hz, CHCH₂), 1.02 (t, 1H, J=7.4 Hz, CHCH₂); ¹³C NMR (75 MHz, δ₆-DMSO) δ 136.0, 135.7, 128.9, 126.5, 58.5, 55.9, 29.9, 23.0, 20.5, 15.2; MS (m/z) 188 (MH⁺, 100).

Synthesis of 3-Ethyl-1-p-tolyl-3-aza-bicyclo[3.1.0]hexane hydrochloride.

¹H NMR (300 MHz, δ₆-DMSO) δ 1.06 (s, 1H, NHCl), 3.92 (dd, 1H, J=11.0 Hz, 5.1 Hz, NCH₃), 3.64 (dd, 1H, J=11.0 Hz, 5.5 Hz, NCH₂), 3.50-3.39 (m, 2H, 2×NCH₂), 3.20 (m, 2H, NCH₂CH₃), 2.29 (s, 3H, ArCH₃), 2.09 (m, 1H, CHCH₂), 1.81 (m, 1H, CHCH₂), 1.29 (t, 3H, J=7.4 Hz, NCH₂CH₃), 1.02 (t, 1H, J=6.6 Hz, CHCH₂); ¹³C NMR (75 MHz, δ₆-DMSO) δ=136.1, 135.7, 128.9, 126.4, 56.7, 54.2, 49.4, 29.5, 22.5, 20.5, 15.5, 10.4; MS (m/z) 202 (MH⁺, 100).

Synthesis of 3-Propyl-1-p-tolyl-3-aza-bicyclo[3.1.0]hexane hydrochloride. ¹H NMR (300 MHz, δ₆-DMSO) δ 11.13 (s, 1H, NHCl), 7.34-7.14 (m, 4H, ArH), 3.90 (dd, 1H, J=11.1 Hz, 5.2 Hz, NCH₂), 3.63 (dd, 1H, J=11.0 Hz, 5.5 Hz, NCH₂), 3.52-3.39 (m, 2H, 2×NCH₂), 3.07 (m, 2H, NCH₂CH₂CH₃), 2.29 (s, 3H, ArCH₃), 2.08 (m, 1H, CHCH₂), 1.84 (m, 1H, CHCH₂), 1.76 (m, 2H, NCH₂CH₂CH₃), 1.01 (t, 1H, J=6.6 Hz, CHCH₂), 0.89 (t, 3H, J=7.3 Hz, NCH₂CH₂CH₃); ¹³C NMR (75 MHz, δ₆-DMSO) δ 136.9, 136.5, 129.7, 127.3, 57.9, 56.7, 55.5, 30.4, 23.4, 21.3, 19.1, 16.3, 11.7; MS (m/z) 216 (MH⁺, 100).

Synthesis of 3-Isopropyl-1-p-tolyl-3-aza-bicyclo[3.1.0]hexane hydrochloride. ¹H NMR (300 MHz, δ₆-DMSO) δ 11.01 (s, 1H, NHCl), 7.21-7.14 (m, 4H, ArH), 3.91 (dd, 1H, J=11.0 Hz, 5.5 Hz, NCH₂), 3.61 (dd, 1H, J=1.0 Hz, 5.5 Hz, NCH₂), 3.54-3.34 (m, 3H, 2×NCH₂, NCH(CH₃)₂), 2.29 (s, 3H, ArCH₃), 2.10 (m, 1H, CHCH₂), 1.90 (t, 1H, J=5.5 Hz, CHCH₂), 1.36 (t, 6H, J=7.0 Hz, NCH(CH₃)₂), 0.98 (t, 1H, J=6.2 Hz, CHCH₂); ¹³C NMR (75 MHz, δ₆-DMSO) δ=0 136.5, 135.9, 129.1, 126.7, 58.3, 56.3, 53.6, 22.9, 20.8, 18.7, 18.6, 15.9; MS (m/z) 216 (MH⁺, 100).

Synthesis of 3-Isobutyl-1-p-tolyl-3-aza-bicyclo[3.1.0]hexane hydrochloride. ¹H NMR (300 MHz, δ₆-DMSO) δ 10.67 (s, 1H, NHCl), 7.21-7.14 (m, 4H, ArH), 4.01 (dd, 1H, J=11.0 Hz, 5.5 Hz, NCH₂), 3.73 (dd, 1H, J=11.1 Hz, 5.6 Hz, NCH₂), 3.52 (m, 2H, 2×NCH₂), 3.05 (t, 2H, J=5.6 Hz, CH₂CH(CH₃)₂), 2.29 (s, 3H, ArCH₃), 2.08 (m, 2H, CH₂CH(CH₃)₂, CHCH₂), 2.00 (t, 1H, J=7.0 Hz, CHCH₂), 1.00 (d, 7H, J=3.3 Hz, NCH₂CH(CH₃)₂, CHCH₂); ¹³C NMR (75 MHz, δ₆-DMSO) δ=144.5, 144.1, 137.2, 134.9, 70.5, 66.5, 64.1, 38.2, 33.4, 31.1. 29.3, 28.9, 24.1; MS (m/z) 230 (MH⁺, 100).

Synthesis of 3-(2-Methoxyethyl)-1-p-tolyl-3-aza-bicyclo[3.1.0]hexane hydrochloride. ¹H NMR (300 MHz, δ₆-DMSO) δ 7.21-7.14 (m, 4H, ArH), 3.90 (dd, 1H, J=11.0 Hz, 5.2 Hz, NCH₂), 3.78 (m, 2H, NCH₂CH₂OCH₃), 3.67 (dd, 1H, J=11.0 Hz, 5.1 Hz, NCH₂), 3.54 (m, 2H, 2×NCH₂), 3.41 (m, 2H, NCH₂CH₂OCH₃), 3.31 (s, 3H, NCH₂CH₂OCH₃), 2.29 (s, 3H, ArCH₃), 2.09 (m, 1H, CHCH₂), 1.75 (t, 1H, J=5.9 Hz, CHCH₂), 1.02 (t, 1H, J=6.6 Hz, CHCH₂); ¹³C NMR (75 MHz, δ₆-DMSO) δ 144.4, 144.2, 137.2, 134.9, 75.2, 66.4, 66.4, 63.9, 61.8, 37.9, 30.9, 28.8, 23.6; MS (m/z) 232 (MH⁺, 100).

Synthesis of 1-p-Tolyl-3-trifluoromethyl-3-aza-bicyclo[3.1.0]hexane hydrochloride. ¹H NMR (300 MHz, δ₆-DMSO) δ 7.14 (s, 4H, ArH), 3.94-3.49 (m, 4H, 4×NCH₂), 2.28 (s, 3H, ArCH₃), 2.01 (m, 1H, CHCH₂), 1.09 (t, 1H, J=5.2 Hz, CHCH₂), 0.89 (t, 1H, J=4.8 Hz, CHCH₂); ¹³C NMR (75 MHz, δ₆-DMSO) δ 155.5, 151.7, 145.4, 143.6, 137.2, 134.7, 60.8, 60.3, 57.7, 57.2, 38.8, 38.2, 31.8, 31.3, 28.8, 26.4, 26.3; MS (m/z) 242 (MH⁺, 5).

Synthesis of 1-p-Tolyl-3-(2,2,2-trifluoroethyl)-3-aza-bicyclo[3.1.0]hexane hydrochloride. ¹H NMR (300 MHz, δ₆-DMSO) δ 7.18-7.12 (m, 4H, ArH), 4.01 (m, 2H, 2×NCH₂), 3.75 (m, 1H, NCH₂), 2.51 (m, 3H, NCH₂CF₃, NCH₂), 2.28 (s, 3H, ArCH₃), 2.00 (m, 1H, CHCH₂), 1.70 (m, 1H, CHCH₂), 0.96 (m, 1H, CHCH₂); ¹³C NMR (75 MHz, δ₆-DMSO) δ 145.32, 143.79, 137.21, 134.82, 67.26, 64.41, 61.95, 61.56, 61.13, 60.71, 38.61, 31.70, 28.85; MS (m/z) 256 (MH⁺, 100).

To produce additional compounds for use within the the methods and compositions of the invention for treating a neuropathic disorder and/or related symptom(s), including multiply aryl-substituted and/or aza-substitued compounds as described above, additional synthetic methods and intermediates are provided herein, as described below.

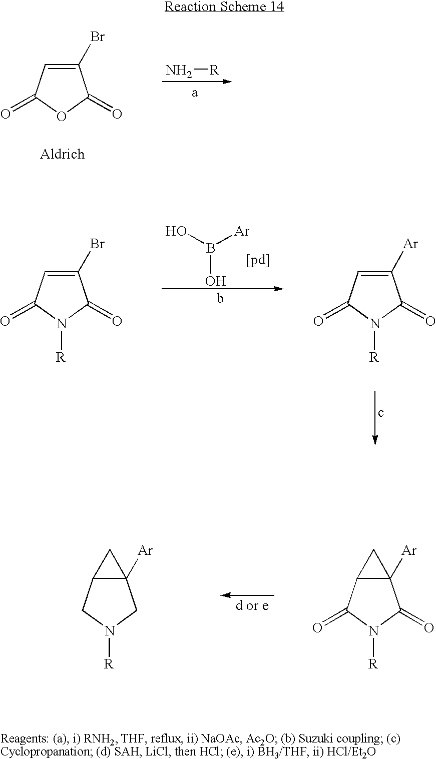

Synthesis of 1-(3,4-dichlorophenyl)-3-oxa-bicyclo[3.1.0]hexane-2,4-dione

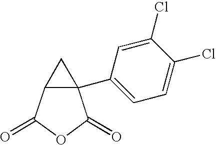

To a stirred solution of 1-(3,4-dichlorophenyl)cyclopropane-1,2-dicarboxylic acid (J. Med. Chem. 1981,24481-490) (28.3 g) in acetyl chloride (142 ml) was heated to reflux for 3 h, cooled to room temperature and evaporated. The oil was dissolved in toluene (100 ml) and evaporated to dryness. This was then repeated a further twice before triturating the semi-solid in hexane (100 ml). The solid was filtered off, washed with hexane and pulled dry under a nitrogen atmosphere to give a brown solid, yield=26.7 g (101%); ¹HNMR (300 MHz, CDCl₃) δ 7.52-7.46 (m, 2H, ArH), 7.27-7.24 (m, 1H, ArH), 3.35-3.30 (m, 1H, CH), 2.13-2.10 (m, 1H, CH), 1.97-1.95 (m, 1H, CH).

### Synthesis of 2-(tert-Butylcarbamoyl)-2-(3,4-dichlorophenyl)cyclopropanecarboxylic acid

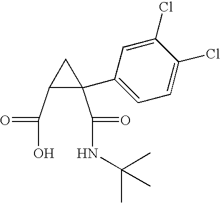

To a stirred solution of the anhydride 1-(3,4-Dichlorophenyl)-3-oxa-bicyclo[3.1.0]hexane-2,4-dione (26.7 g) in tetrahydrofuran (THF) (365 ml) was added tert-butylamine (23 ml) with the temperature kept below 20° C. The suspension was then stirred at room temperature for 1 h where thin-layer chromatography (TLC) (50% ethyl acetate in hexanes) indicated complete reaction. The solvent was evaporated off and the resultant sticky mass used crude in the next reaction.

### Synthesis of 3-tert-Butyl-1-(3,4-dichlorophenyl)-3-aza-bicyclo[3.1.0]hexane-2,4-dione

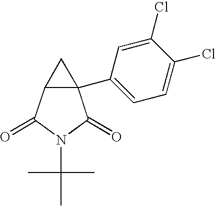

A stirred suspension of the 2-(tert-Butylcarbamoyl)-2-(3,4-dichlorophenyl)cyclopropanecarboxylic acid and sodium acetate (4.3 g) in acetic anhydride (145 ml) was heated to reflux for 4 h where TLC (50% ethyl acetate in hexanes) indicated complete reaction so the solvent was evaporated off and the oil absorbed onto silica (49.7 g). The product was then purified by column chromatography [SiO₂ (503.7 g): (10% EtOAc in hexanes)] to give the required material as a yellow oil, in a yield of 23.7 g (73%); ¹HNMR (300 MHz, CDCl₃) δ 7.52-7.46 (m, 2H, ArH), 7.23-7.20 (m, 1H, ArH), 2.64-2.60 (m, 1H, CH), 1.72-1.66 (m, 2H, CH), 1.52 (s, 9H, Bu^{t})

### Synthesis of 3-tert-Butyl-1-(3,4-dichlorophenyl)-3-aza-bicyclo[3.1.0]hexane-2-one

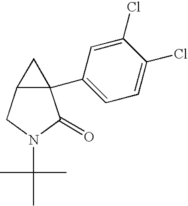

To a stirred solution of 3-tert-Butyl-1-(3,4-dichlorophenyl)-3-aza-bicyclo[3.1.0]hexane-2,4-dione (23.7 g) in THF (395 ml) at 5° C. was added a solution of borane in THF (1M; 304 ml) with the temperature kept below 5° C. The solution was then heated to reflux for 2 h where TLC (20% ethyl acetate in hexanes) indicated complete reaction. The solution was cooled to 0° C. and quenched by the addition of dilute HCl (6M; 400 ml) with the temperature kept below 10° C. The THF was evaporated off and the white solid filtered off and dried at 45° C. in vacuo overnight, yielding 17.0 g (75%) of the desired product;

¹HNMR (300 MHz, CDCl₃) δ 7.71 (d, 1H, J=2.4 Hz, ArH), 7.57 (d, 1H, J=8.4 Hz, ArH), 7.36 (dd, 1H, J=8.4 Hz, J=2.4 Hz, ArH), 4.86 (br s, 2H, CH₂), 3.69-3.63 (m, 1H, CH), 3.46-3.43 (m, 1H, CH), 2.37-2.31 (m, 1H, CH), 1.45-1.42 (m, 1H, CH), 1.32 (s, 9H, Bu^{t}) MS (m/z) 299 (MH⁺, 100).

### Synthesis of 3-tert-Butyl-1-(3,4-dichlorophenyl)-3-aza-bicyclo[3.1.0]hexane

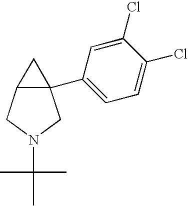

To a stirred solution of 3-tert-Butyl-1-(3,4-dichlorophenyl)-3-aza-bicyclo[3.1.0]hexane-2-one (15.1 g) in THF (270 ml) was added a solution of borane in THF (1M; 203 ml) at 20° C. The solution was then heated to reflux for 16 h where TLC (20% ethyl acetate in hexanes) indicated incomplete reaction so the solution was cooled to room temperature a further portion of borane in THF (1M; 130 ml) was added at 20° C. The solution was then again heated to reflux and held for 24 h. TLC indicated approximately 50% reaction so the solution was cooled to 0° C. and quenched by the addition of dilute HCl (6M; 400 ml) with the temperature kept below 10° C. The THF was evaporated off, the white solid filtered off, and the aqueous extracted with ethyl acetate (3×250 ml). The aqueous was basified with NaOH (5M; 500 ml) and the product extracted into ether (3×200 ml), dried (MgSO₄) and evaporated to give a colourless oil, in a yield of 5.9 g (41%). The crude 3-tert-Butyl-1-(3,4-dichlorophenyl)-3-aza-bicyclo[3.1.0]hexane was added to a solution of maleic acid (2.3 g) in methanol (11.5 ml) and stored at −20° C. overnight. The solid was filtered off, washed with methanol (2.5 ml) and dried at 45° C. in vacuo overnight, yielding 1-(3,4-dichloro-phenyl)-3-tert-butyl-3-aza-bicyclo[3.1.0]-hexane maleate salt 1.1 g (5%); ¹HNMR (300 MHz, CDCl₃) δ 7.31-7.19 (m, 2H, ArH), 6.95-6.91 (m, 1H, ArH), 3.28 (d, 1H, J=8.4 Hz, CH), 3.10 (d, 1H, J=8.4 Hz, CH), 2.48-2.40 (m, 4H, CH), 1.68-1.62 (m, 1H, CH), 1.47-1.33 (m, 5H, CH), 0.92-0.87 (m, 3H, CH₃), 0.77-0.74 (m, 1H, CH) MS (m/z) 284 (M⁺, 100)

### Synthesis of 1-(3,4-dichloro-phenyl)-3-n-butyl-3-aza-bicyclo[3.1.0]-hexane

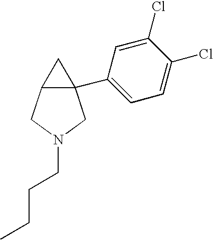

To a stirred solution of 1-(3,4-dichlorophenyl)-3-aza-bicyclo[3.1.0]hexane-2,4-dione) (15.8 g) in N,N-Dimethylformamide (DMF) (63 ml) was added sodium hydride (60 wt. % in oil; 2.5 g) with the temperature kept below 20° C. The suspension was then stirred at room temperature for 20 mins before 1-bromobutane (9.9 ml) was added. The solution was then stirred at room temperature for 24 h when TLC (20% ethyl acetate in hexanes) indicated complete reaction. The solution was quenched into water (500 ml), extracted with ether (2×250 ml) and the extracts washed with water (2×250 ml), saturated brine (2×250 ml), dried (MgSO₄) and evaporated, yielding 15.6 g of 3-Butyl-1-(3,4-dichlorophenyl)-3-aza-bicyclo[3.1.0]hexane-2,4-dione (81%). The imide (3-Butyl-1-(3,4-dichlorophenyl)-3-aza-bicyclo[3.1.0]hexane-2,4-dione) was dissolved in THF (310 ml) and a solution of borane in THF (1M; 225 ml) was added with the temperature kept below 5° C. The solution was then heated to reflux for 4 h where TLC (20% ethyl acetate in hexanes) indicated complete reaction. The solution was cooled to 0° C. and quenched by the addition of dilute HCl (6M; 200 ml) with the temperature kept below 10° C. The solution was then extracted with ether (2×200 ml), the aqueous basified with sodium hydroxide (5M; 480 ml), extracted with ether (3×150 ml), the extracts combined, dried (MgSO₄) and evaporated, to give a crude yield of 3.2 g. The oil was added to HCl in ether (2M; 20 ml), stored overnight at −20° C. and the resultant solid filtered off and washed with ether (2×10 ml). TLC (20% ethyl acetate in hexanes) indicated two components so the solid was dissolved in water (50 ml) basified with solid K₂CO₃ to pH 10 and extracted with ether (3×100 ml). The extracts were dried (MgSO₄) and evaporated. The product was then purified by chromatography [SiO₂ (22.7 g): (25% EtOAc in hexanes)] to give the required material as a yellow oil, 0.7 g (5%); ¹HNMR (300 MHz, CDCl₃) δ 7.16-7.06 (m, 4H, ArH), 3.97 (t, 1H, J=6.3 Hz, NCH₂), 3.78 (s, 3H, NCH₂), 2.34 (s, 3H, ArCH₃), 1.87 (m, 1H, CHCH₂), 1.19 (t, 1H, J=5.5 Hz, CHCH₂), 0.87 (m, 1H, CHCH₂) MS (m/z) 188 (MH⁺, 100)

### Synthesis of 2-(Propylcarbamoyl)-2-(3,4-dichlorophenyl)cyclopropanecarboxylic acid

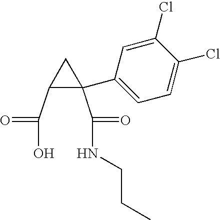

To a stirred solution of 1-(3,4-Dichlorophenyl)-3-oxa-bicyclo[3.1.0]hexane-2,4-dione (12.8 g) in THF (175 ml) was added n-propylamine (8.6 ml) with the temperature kept below 20° C. The suspension was then stirred at room temperature for 1 h where TLC (50% ethyl acetate in hexanes) indicated complete reaction. The solvent was evaporated off and the resultant sticky mass used crude in the next reaction.

### Synthesis of 3-Propyl-1-(3,4-dichlorophenyl)-3-aza-bicyclo[3.1.0]hexane-2,4-dione

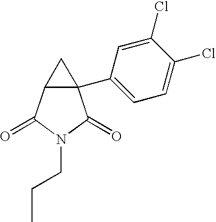

A stirred suspension of the above amide 2-(Propylcarbamoyl)-2-(3,4-dichlorophenyl)cyclopropanecarboxylic acid and sodium acetate (4.1 g) in acetic anhydride (68 ml) was heated to reflux for 4 h where TLC (50% ethyl acetate in hexanes) indicated complete reaction so the solvent was evaporated off and the oil absorbed onto silica (14.4 g). The product was purified by column chromatography [SiO₂ (147.2 g): (20% EtOAc in hexanes)] to give the required material as a yellow oil, 4.6 g (31% over three steps).

### Synthesis of 1-(3,4-Dichloro-phenyl)-3-n-propyl-3-aza-bicyclo[3.1.0]-hexane hydrochloride

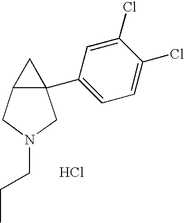

To a stirred solution of 3-Propyl-1-(3,4-dichlorophenyl)-3-aza-bicyclo[3.1.0]hexane-2,4-dione (4.6 g) in THF (92 ml) at 5° C. was added a solution of borane in THF (1M; 69 ml) with the temperature kept below 5° C. The solution was then heated to reflux for 4 h where TLC (20% ethyl acetate in hexanes) indicated complete reaction. The solution was cooled to 0° C. and quenched by the addition of dilute HCl (6M; 250 ml) with the temperature kept below 10° C. The THF was evaporated off and the aqueous extracted with ether (2×250 ml). The aqueous was basified with NaOH (5M; 250 ml) and the product extracted into ether (2×150 ml), dried (MgSO₄) and evaporated to give a colourless oil, 2.3 g (17%). The oil was dissolved in ether (30 ml) and a solution of HCl in ether (2M; 30 ml) was added. The suspension was then stored at −20° C. overnight. The solid was filtered off, washed with ether (20 ml) and dried at 40° C. in-vacuo overnight, yield=1.3 g (50%); ¹HNMR (300 MHz, CDCl₃) δ 12.56 (br s, 1H, NH⁺), 7.66-7.55 (m, 1H, ArH), 7.26 (s, 1H, ArH), 7.02-6.99 (m, 1H, ArH), 4.12-4.10 (m, 1H, CH), 4.09-3.90 (m, 1H, CH), 3.18-3.01 (m, 4H, CH₂), 2.40-2.36 (m, 1H, CH), 2.02-1.98 (m, 3H, CH), 1.18-0.95 (m, 4H, CH₂) MS (m/z) 270 (MH⁺, 100)

### Chiral Separation of 1-(3,4-dichloro-phenyl)-3-methyl-3-aza-bicyclo-[3.1.0]hexane

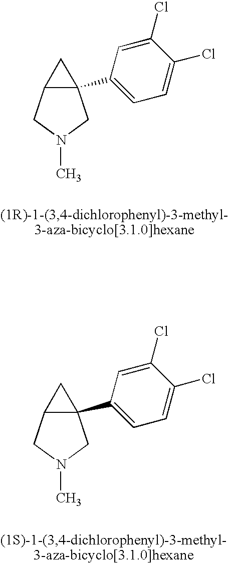

A solution of racemic 1-(3,4-dichloro-phenyl)-2-oxo-3-methyl-3-aza-bicyclo-[3.1.0]hexane (0.75 g) was prepared using methanol (10 mL). This solution was then injected onto a CHIRALCEL® OD-H 5 μm column and an isocratic run was started with UV monitoring at 275 nm, flow rate 60 mL/min; Mobile Phase: 95:5 CO₂/MeOH+2% DEA. Peaks were collected separately and concentrated to dryness under reduced pressure to give the desired elutes as first eluting enantiomer and second eluting enantiomer.

SFC Preparative Method:

Column: 250×20 mm CHIRALCEL® OD-H 5 μm
Mobile phase: 95:5 CO₂/MeOH+2% DEA
Flow rate: 60 ml/min
Detection: UV 275 nm
Temperature: 15° C.
Outlet pressure: 150 bar
An HPLC analytical method was developed, in order to control the purity of the collected fractions.
   HPLC Analytical Method:
Column: 250×4.6 mm CHIRALCEL® OD-H 5 μm
Mobile phase: 98:2:0.1 n-heptane/2-PrOH/DEA
Flow rate: 0.5 ml/min
Detection: DAD 250 nm
Temperature: 25° C.

### Synthesis of 1-(3,4-dichloro-phenyl)-3-methyl-3-aza-bicyclo[3.1.0]-hexane hydrochloride first eluting enantiomer

To a stirred solution of the first elute 1-(3,4-dichloro-phenyl)-3-methyl-3-aza-bicyclo[3.1.0]-hexane (117 mg) in ether (5 ml) was added a solution of HCl in ether (2M; 5 ml). The suspension was then stored at −20° C. overnight. The solid was filtered off, washed with ether (5 ml) and dried in-vacuo overnight, yield=57.8 mg (43%); ¹HNMR (300 MHz, CDCl₃) δ 12.85 (s, 1H, NH⁺), 7.43-7.03 (m, 3H, ArH), 4.16-3.97 (m, 1H, CH), 3.31-2.93 (m, 3H, CH), 2.35 (s, 1H, CH), 2.05 (s, 1H, CH), 1.57 (s, 3H, CH₃) MS (m/z) 242 (MH⁺, 100)

### Synthesis of 1-(3 4-dichloro-phenyl)-3-methyl-3-aza-bicyclo[3.1.0]-hexane hydrochloride second eluting enantiomer

To a stirred solution of the second elute 1-(3,4-dichloro-phenyl)-3-methyl-3-aza-bicyclo[3.1.0]-hexane (143 mg) dissolved in ether (5 ml) was added a solution of HCl in ether (2M; 5 ml). The suspension was then stored at −20° C. overnight. The solid was filtered off, washed with ether (5 ml) and dried in-vacuo overnight, yield=59.4 mg (36%); ¹HNMR (300 MHz, CDCl₃) δ 12.85 (s, 1H, NH⁺), 7.43-7.03 (m, 3H, ArH), 4.16-3.97 (m, 1H, CH), 3.31-2.93 (m, 3H, CH), 2.35 (s, 1H, CH), 2.05 (s, 1H, CH), 1.57 (s, 3H, CH₃) MS (m/z) 242 (MH⁺, 100)

The following description illustrates an exemplary synthetic method for producing compounds of the invention, which is illustrative of general synthetic Scheme 14, as described above.

### Synthesis of N-Methyl bromomaleimide

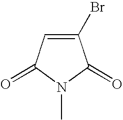

A solution of bromomaleic anhydride (aldrich) (52.8 g, 0.298 mol) in diethyl ether (250 mL) was cooled to 5° C. A 2 M solution of methylamine in THF (298 mL, 0.596 mol, 2 eq.) was added dropwise over 1 hour and the reaction stirred for a further 30 minutes, maintaining the temperature below 10° C. The resulting precipitate was filtered, washed with diethyl ether (2×100 mL) and air-dried for 30 minutes then suspended in acetic anhydride (368 mL) and sodium acetate (12.2 g, 0.149 mol, 0.5 eq.) added. The reaction was heated to 60° C. for 2 hours then solvent removed in vacuo. The residue was taken up in DCM (500 mL) and washed with saturated sodium bicarbonate solution (2×500 mL) and water (2×300 mL). Organics were dried over MgSO₄ (89 g), filtered and reduced in vacuo. The resulting oil was azeotroped with toluene (4×100 mL) to give N-methyl bromomaleimide as a beige solid (41.4 g, 73 %); ¹H NMR (300 MHz, CDCl₃) δ 6.95 (1H, s, CH), 3.07 (3H, s, CH₃N).

### Synthesis of N-Methyl-(3-chloro-4-fluorophenyl)maleimide

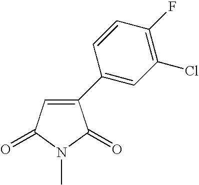

N-Methyl bromomaleimide (20.3 g, 0.107 mol), 3-chloro-4-fluorobenzene boronic acid (20.5 g, 0.117 mol, 1.1 eq.), cesium fluoride (35.8 g, 0.235 mol, 2.2 eq.) and 1,1′-bis-diphenylphosphinoferrocene palladium chloride (4.3 g, 0.005 mol, 5 mol %) were suspended in 1,4-dioxane and stirred at room temperature for 1 hour then heated to 40° C. for 2 hours. The reaction was filtered and solvents removed in vacuo. The dark brown residue was taken up in DCM (100 mL) then filtered through silica (100 g), eluting with 1.5 L of DCM. Solvents were removed in vacuo and the resulting solid slurried in hexane (100 mL) and filtered. The cake was washed with a further portion of hexane (100 mL) and dried to give N-methyl-(3-chloro-4-fluorophenyl)maleimide as a pale orange solid, (19.0 g, 74%); ¹H NMR (300 MHz, CDCl₃) 8.04-8.01 (1H, dd, J=6.9, 2.1 Hz, ArH), 7.86-7.81 (1H, m, ArH), 7.22-7.19 (1H, t, J=9 Hz, ArH), 6.71 (1H, s, CH), 3.07 (3H, s, CH₃); MS (m/z) 239 [MH⁺] (60), 241 (20).

### Synthesis of 1-(3-chloro-4-fluorophenyl)-3-methyl-3-aza-bicyclo[3.1.0]hexane-2,4-dione

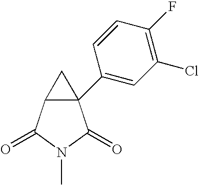

Trimethylsulphoxonium chloride (2.5 g, 0.019 mol, 1.2 eq.) and sodium hydride (0.8 g of a 60% dispersion in mineral oil, 0.019 mol, 1.2 eq.) were suspended in THF (180 mL) and heated at reflux (66° C.) for 2.5 hours. The reaction was cooled to 50° C. and a solution of N-methyl-(3-chloro-4-fluorophenyl)maleimide (6) (3.8 g, 0.016 mol, 1 eq.) in THF (20 mL) added in one portion. The reaction was heated at 50° C. for 2 hours and then cooled to room temperature. IMS (5 mL) was added to quench any unreacted sodium hydride and the solvent removed in vacuo. The residue was taken up in DCM (150 mL) and washed with water (4×150 ml), dried over MgSO₄ (32 g), filtered and solvents removed in vacuo. The reaction was purified by column chromatography (60 g silica, eluting with 4:1 hexane:ethyl acetate (500 mL)). Solvents were removed in vacuo to give 1-(3-chloro-4-fluorophenyl)-3-methyl-3-aza-bicyclo[3.1.0]hexane-2,4-dione as a pale yellow solid (1.6 g, 40%); ¹H NMR (300 MHz, CDCl₃) 7.45-7.43 (1H, dd, J=6.6, 2.1 Hz, ArH), 7.30-7.27 (1H, m, ArH), 7.16-7.10 (1H, t, J=8.7 Hz, ArH), 2.91 (3H, s, CH₃), 2.74-2.70 (1H, dd, J=8.1, 3.9 Hz, CH), 1.87-1.84 (1H, t, J=4.2 Hz, CH), 1.81-1.76 (1H, dd, J=8.1, 4.8 Hz, CH).

### Synthesis of 1-(3-chloro-4-fluorophenyl)-3-methyl-3-aza-bicyclo[3.1.0]hexane

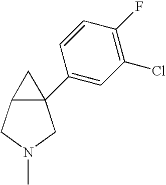

Borane (1 M complex in THF, 31.5 mL, 0.032 mol, 5 eq.) was cooled to <0° C. and a solution of (7) (1.6 g, 0.006 mol) in THF (30 mL) added dropwise, maintaining the temperature <0° C. The reaction was warmed to room temperature for 15 minutes then heated to reflux (67° C.) for 2.5 hours. The reaction was cooled to <0° C. and quenched with the dropwise addition of 6 M HCl (14 mL, temperature maintained <0° C.). Solvents were removed in vacuo and the resulting white residue partitioned between 5 M NaOH (50 mL) and diethyl ether (50 mL). The aqueous layer was re-extracted with a further 50 mL of diethyl ether then combined organics washed with water (3×75 mL), dried over MgSO₄ (14 g), filtered and solvents removed in vacuo to give a yellow oil. A 2 M solution of HCl in diethyl ether (12 mL) was added and the reaction cooled to <0° C. to precipitate out the HCl salt. The solid was washed with HCl/ether (3×6 mL) to give 1-(3-chloro-4-fluorophenyl)-3-methyl-3-aza-bicyclo[3.1.0]hexane as a pale yellow solid, 774 mg, 47% yield; ¹H NMR (300 MHz, CDCl₃) 12.74 (1H, br-s, N⁺H), 7.26-7.24 (1H, m, ArH), 7.15-7.04 (2H, m, ArH), 4.12-4.06 (1H, dd, J=10.8, 5.4 Hz, CH₂), 3.96-3.90 (1H, dd, J=11.1, 5.1 Hz, CH₂), 3.36-3.30 (1H, m, CH₂), 3.24-3.18 (1H, t, J=9.3 Hz, CH₂), 2.91 (3H, s, CH₃), 2.29-2.26 (1H, dd, J=6.9, 4.8 Hz, CH), 2.03-1.97 (1H, q, J=4.2 Hz, CH), 1.22-1.17 (1H, m, CH); MS (m/z) 226 [MH⁺] (100), 228 [MH⁺²].

With regard to the foregoing synthetic schemes, and as otherwise used herein unless specified differently, Ar denotes a phenyl or other aromatic group having multiple substitutions on the aryl ring, and R is selected from, for example, hydrogen, C₁₋₆ alkyl, halo(C₁₋₆)alkyl, C₃₋₉ cycloalkyl, C₁₋₅ alkoxy(C₁₋₆)alkyl, carboxy(C₁₋₃)alkyl, C₁₋₃ alkanoyl, carbamate, halo(C₁₋₃)alkoxy(C₁₋₆)alkyl, C₁₋₃ alkylamino(C₁₋₆)alkyl, and di(C₁₋₃)alkylamino(C₁₋₆)alkyl, cyano(C₁₋₆)alkyl, methyl, ethyl, trifluoromethyl, trifluoroethyl and 2-methoxyethyl.

For the purposes of further describing the invention, including the novel compounds and synthetic methods disclosed herein, the following terms and definitions are provided by way of example.

The term “halogen” as used herein refers to bromine, chlorine, fluorine or iodine. In one embodiment, the halogen is chlorine. In another embodiment, the halogen is bromine.

The term “hydroxy” as used herein refers to —OH or —O⁻.

The term “alkyl” as used herein refers to straight- or branched-chain aliphatic groups containing 1-20 carbon atoms, often 1-7 carbon atoms and in certain embodiments 1-4 carbon atoms. This definition applies as well to the alkyl portion of alkoxy, alkanoyl and aralkyl groups. In one embodiment, the alkyl is a methyl group.

The term “alkoxy” includes substituted and unsubstituted alkyl, alkenyl, and alkynyl groups covalently linked to an oxygen atom. In one embodiment, the alkoxy group contains 1 to 4 carbon atoms. Embodiments of alkoxy groups include, but are not limited to, methoxy, ethoxy, isopropyloxy, propoxy, butoxy, and pentoxy groups. Embodiments of substituted alkoxy groups include halogenated alkoxy groups. In a further embodiment, the alkoxy groups can be substituted with groups such as alkenyl, alkynyl, halogen, hydroxyl, alkylcarbonyloxy, phenylcarbonyloxy, alkoxycarbonyloxy, phenyloxycarbonyloxy, carboxylate, alkylcarbonyl, phenylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, phenylamino, diphenylamino, and alkylphenylamino), acylamino (including alkylcarbonylamino, phenylcarbonylamino, carbamoyl and ureido), amidino, imino, sulfhydryl, alkylthio, phenylthio, thiocarboxylate, sulfates, alkylsulfinyl, sulfonato, sulfamoyl, sulfonamido, nitro, trifluoromethyl, cyano, azido, heterocyclyl, alkylphenyl, or an aromatic or heteroaromatic moieties. Exemplary halogen substituted alkoxy groups include, but are not limited to, fluoromethoxy, difluoromethoxy, trifluoromethoxy, chloromethoxy, dichloromethoxy, and trichloromethoxy.

The term “nitro”, as used herein alone or in combination refers to a —NO₂ group.

The term “amino” as used herein refers to the group —NRR′, where R and R′ may independently be hydrogen, alkyl, phenyl, alkoxy, or heterophenyl. The term “aminoalkyl” as used herein represents a more detailed selection as compared to “amino” and refers to the group —NRR′, where R and R′ may independently be hydrogen or (C₁₋₄)alkyl.

The term “trifluoromethyl” as used herein refers to —CF₃.

The term “trifluoromethoxy” as used herein refers to —OCF₃.

The term “cycloalkyl” as used herein refers to a saturated cyclic hydrocarbon ring system containing from 3 to 7 carbon atoms that may be optionally substituted. Exemplary embodiments include, but are not limited to, cyclopropyl, cyclobutyl, cyclopentyl and cyclohexyl. In certain embodiments, the cycloalkyl group is cyclopropyl. In another embodiment, the (cycloalkyl)alkyl groups contain from 3 to 7 carbon atoms in the cyclic portion and 1 to 4 carbon atoms in the alkyl portion. In certain embodiments, the (cycloalkyl)alkyl group is cyclopropylmethyl. The alkyl groups are optionally substituted with from one to three substituents selected from the group consisting of halogen, hydroxy and amino.

The terms “alkanoyl” and “alkanoyloxy” as used herein refer, respectively, to —C(O)-alkyl groups and —O—C(O)-alkyl groups, each optionally containing 2-5 carbon atoms. Specific embodiments of alkanoyl and alkanoyloxy groups are acetyl and acetoxy, respectively.

The term “aroyl,” as used alone or in combination herein, refers to an phenyl radical derived from an aromatic carboxylic acid, such as optionally substituted benzoic or naphthoic acids.

The term “aralkyl” as used herein refers to a phenyl group bonded to the 4-pyridinyl ring through an alkyl group, often one containing 1-4 carbon atoms. An exemplary aralkyl group is benzyl.

The term “nitrile” or “cyano” as used herein refers to the group —CN.

The term “pyrrolidine-1-yl” as used herein refers to the structure:

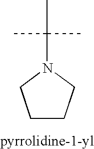

The term “morpholino” as used herein refers to the structure:

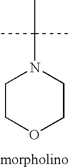

The term “dialkylamino” refers to an amino group having two attached alkyl groups that can be the same or different.

The term “alkenyl” refers to a straight or branched alkenyl group of 2 to 10 carbon atoms having 1 to 3 double bonds. Exemplary embodiments include ethenyl, 1-propenyl, 2-propenyl, 1-methylethenyl, 1-butenyl, 2-butenyl, 3-butenyl, 2-methyl-2-propenyl, 1-pentenyl, 2-pentenyl, 4-pentenyl, 3-methyl-2-butenyl, 1-hexenyl, 2-hexenyl, 1-heptenyl, 2-heptenyl, 1-octenyl, 2-octenyl, 1,3-octadienyl, 2-nonenyl, 1,3-nonadienyl, 2-decenyl, etc.

The term “alkynyl” as used herein refers to a straight or branched alkynyl group of 2 to 10 carbon atoms having 1 to 3 triple bonds. Exemplary alkynyls include, but are not limited to, ethynyl, 1-propynyl, 2-propynyl, 1-butynyl, 2-butynyl, 3-butynyl, 1-pentynyl, 2-pentynyl, 4-pentynyl, 1-octynyl, 6-methyl-1-heptynyl, and 2-decynyl.

The term “hydroxyalkyl” alone or in combination, refers to an alkyl group as previously defined, wherein one or several hydrogen atoms, often one hydrogen atom, has been replaced by a hydroxyl group. Examples include hydroxymethyl, hydroxyethyl and 2-hydroxyethyl.

The term “aminoalkyl” as used herein refers to the group —NRR′, where R and R′ may independently be hydrogen or (C₁-C₄)alkyl.

The term “alkylaminoalkyl” refers to an alkylamino group linked via an alkyl group (i.e., a group having the general structure—alkyl-NH-alkyl or —alkyl-N(alkyl)(alkyl)). Such groups include, but are not limited to, mono- and di-(C₁-C₈ alkyl)aminoC₁-C₈ alkyl, in which each alkyl may be the same or different.

The term “dialkylaminoalkyl” refers to alkylamino groups attached to an alkyl group. Examples include, but are not limited to, N,N-dimethylaminomethyl, N,N-dimethylaminoethyl N,N-dimethylaminopropyl, and the like. The term dialkylaminoalkyl also includes groups where the bridging alkyl moiety is optionally substituted.

The term “haloalkyl” refers to an alkyl group substituted with one or more halo groups, for example chloromethyl, 2-bromoethyl, 3-iodopropyl, trifluoromethyl, perfluoropropyl, 8-chlorononyl and the like.

The term “carboxyalkyl” as used herein refers to the substituent —R′—COOH wherein R′ is alkylene; and carbalkoxyalkyl refers to —R′—COOR wherein R′ and R are alkylene and alkyl respectively. In certain embodiments, alkyl refers to a saturated straight- or branched-chain hydrocarbyl radical of 1-6 carbon atoms such as methyl, ethyl, n-propyl, isopropyl, n-butyl, t-butyl, n-pentyl, 2-methylpentyl, n-hexyl, and so forth. Alkylene is the same as alkyl except that the group is divalent.

The term “alkoxyalkyl” refers to an alkylene group substituted with an alkoxy group. For example, methoxyethyl [CH₃OCH₂CH₂—] and ethoxymethyl (CH₃CH₂OCH₂—] are both C₃ alkoxyalkyl groups.

The term “carboxy”, as used herein, represents a group of the formula —COOH.

The term “alkanoylamino” refers to alkyl, alkenyl or alkynyl groups containing the group —C(O)— followed by —N(H)—, for example acetylamino, propanoylamino and butanoylamino and the like.

The term “carbonylamino” refers to the group —NR—CO—CH₂—R′, where R and R′ may be independently selected from hydrogen or (C₁-C₄)alkyl.

The term “carbamoyl” as used herein refers to —O—C(O)NH₂.

The term “carbamyl” as used herein refers to a functional group in which a nitrogen atom is directly bonded to a carbonyl, i.e., as in —NRC(═O)R′ or —C(═O)NRR′, wherein R and R′ can be hydrogen, alkyl, substituted alkyl, alkenyl, substituted alkenyl, alkoxy, cycloalkyl, phenyl, heterocyclo, or heterophenyl.

The term “alkylsulfonylamino” refers to refers to the group —NHS(O)₂Rₐ wherein Rₐ is an alkyl as defined above.

In certain detailed embodiments, the methods and compositions of the invention for treating or preventing neuropathic disorders and/or related symptoms employ a compound or formulation comprising a 1-aryl-3-azabicyclo[3.1.0]hexane having at least one substituent on the phenyl/aryl ring.

In alternate embodiments, the methods and compositions of the invention for treating or preventing neuropathic disorders and/or related symptoms employ a 1-aryl-3-azabicyclo[3.1.0]hexane having two or more substituents on the phenyl/aryl ring.

In other detailed embodiments, the methods and compositions of the invention for treating or preventing neuropathic disorders and/or related symptoms employ a 1-aryl-3-azabicyclo[3.1.0]hexane having an aza substitution on the nitrogen at the ‘3’ position.

In additional detailed embodiments of the invention, the methods and compositions of the invention for treating or preventing neuropathic disorders and/or related symptoms employ bi-substituted 1-aryl-3-azabicyclo[3.1.0]hexanes having at least one substitution on the aryl ring, as well as an aza substitution on the nitrogen at the ‘3’ position.

Useful 1-aryl-3-azabicyclo[3.1.0] hexanes for use within the methods and compositions of the invention for treating or preventing neuropathic disorders and/or related symptoms include the substituted and bi-substituted 1-aryl-3-azabicyclo[3.1.0]hexanes compounds described herein, as well as, without limitation, active, pharmaceutically acceptable salts, polymorphs, solvates, hydrates and/or prodrugs of these compounds, or combinations thereof.

The methods and compositions of the invention are effective to treat and/or prevent a variety of symptoms and conditions associated with neuropathic disorders in mammalian subjects. A broad range of mammalian subjects, including human subjects, are amenable for treatment using the formulations and methods of the invention. These subjects include, but are not limited to, human and other mammalian subjects suffering from any one or combination of the following disorders, conditions and/or symptoms: diabetic neuropathy; diabetic peripheral neuropathy (including distal symmetrical polyneuropathy); post-herpetic neuralgia, trigeminal neuralgia; neuropathy associated with alcoholism; sciatica, post-stroke pain; multiple sclerosis; shingles; idiopathic or post-traumatic neuropathy and mononeuritis; HIV-associated neuropathy; cancer; carpal tunnel syndrome; neuropathy associated with Fabry's disease; vasculitic neuropathy; neuropathy associated with Guillain-Barre syndrome; chronic low back pain; iatrogenic-induced neuropathies (e.g., as induced by the anti-tumor agents taxol and paclitaxil, and by certain anti-retroviral drugs), dietary or absorption abnormality; spinal cord injury; vitamin deficiencies; heavy metal poisoning; complex regional pain syndrome; fibromyalgia; peripheral nerve trauma; entrapment neuropathy; nerve transection; Wallenberg's syndrome; connective tissue disease; plexus irradiation; ischemic irradiation; hematomyelia; dyscraphism; tumor compression; arteriovenuous malformation; syphilitic myelitis; commissural myelotomy; arachnoiditis; root avulsion; prolapsed disk compression; lumbar and cervical pain; reflex sympathic dystrophy; phantom limb syndrome, among other chronic neuropathic syndromes, conditions and symptoms.

As noted above, a principal adverse symptom associated with neuropathies is neuropathic pain, which is typically associated with aberrant somatosensory processing in the peripheral or central nervous system. In contrast to nociceptive pain, neuropathic pain is frequently described as “burning”, “electric”, “tingling”, and “shooting” in nature. Additionally, whereas nociceptive pain is mediated by stimulation of peripheral A-delta and C-polymodal pain receptors (e.g., by histamine bradykinin, substance P, etc.), neuropathic pain is typically caused at least in part by damage to, or pathological changes in, peripheral and/or central nerves. Examples of pathological changes to nerves include prolonged peripheral or central neuronal sensitization, central sensitization related damage to nervous system inhibitory functions, and abnormal interactions between the somatic and sympathetic nervous systems.

Neuropathic symptoms that may be characterized as “neuropathic pain” and which are treatable or preventable using the formulations and methods of the invention include, for example: allodynia (painful response to a non-noxious stimulus); tactile allodynia (painful response to normally non-noxious touch); hyperalgesia (heightened or extreme sensitivity to painful stimuli); thermal hyperalgesia (exaggerated painful response to noxious temperatures); mechanical hyperalgesia (exaggerated painful response to normally noxious body movement); paraesthesias (abnormal sensations such as tingling, burning, pricking or tickling); hyperesthesia (enhanced sensitivity to a natural stimuli); and dysesthesias (disagreeable sensations produced by ordinary stimuli).

It is currently believed that after nerve injury, peripheral nerves begin to degenerate, starting at the site of injury and progressing to the nerve terminal. This process, often referred to as Wallerian degeneration, has been characterized extensively in animal models (e.g., the spinal nerve ligation (Chung) model, which is widely accepted in the art as a useful model of neuropathic conditions and for selecting and characterizing effective drugs to treat symptoms associated with neuropathies in mammals, including humans). During degeneration, the axoplasm gradually disintegrates and the axolemma fragments. Schwann cells and macrophages phagocytose myelin debris. This process activates secretion of a series of known and unknown cytokines and growth factors, including interferons, tumor necrosis factor-alpha (TNFα), nerve growth factor (NGF), and interleukins. These cytokines and growth factors influence the structure and function of both adjacent and distal tissues, including by inducing apoptosis in a number of peripheral cells and production of trophic factors required for regeneration of both nerve and peripheral cells.

Development of hyperalgesia in nerve injured animals is thought to arise from early electrophysiological events like “injury discharge” that alters neuronal influx of calcium to activate kinases such as protein kinase A and C, and the extracellular regulated kinases (ERKs), leading to proliferation, chemotaxis and other cellular activation at the injury site and physiological changes at the cell body; and intermediate events such as retrograde injury signals that include target derived growth factors and cytokines. These events can occur from hours to weeks after nerve injury resulting in pain and hypersensitivity for the duration of the process. Primary hyperalgesia, caused by sensitization of C-fibers, occurs immediately within the area of the injury. Secondary hyperalgesia, caused by sensitization of dorsal horn neurons, occurs in undamaged areas surrounding the injury.

Trophic factors such as nerve growth factor (NGF) and tumor necrosis factor-α (TNF-α) produced by Schwann cells and invading macrophages after nerve injury are correlated with the onset of hyperalgesia. Thus, changes in endogenous, systemic, or local levels of these and other growth factors and cytokines will often be useful as diagnostic indices to select subjects amenable for treatment according to the methods and compositions of the invention, or to manage or customize treatments according to the invention on a patient-specific basis. Interestingly, both NGF and TNF-α also have positive regenerative effects on damaged nerves, but cause pain in both undamaged and damaged nerves and result in thermal hyperalgesia and mechanical allodynia in non-injured animals. Similar responses are seen in humans.

As noted above, analgesics, including NSAIDs and opiates, which are effective for treating general nociceptive pain, are rarely effective for neuropathic pain (The Lancet, 353:1959-1966, 1999). For example, morphine has a strong analgesic effect on nociceptive pain, but does not exhibit remarkable/sufficient activity for alleviating neuropathic pain. In fact, resistance to morphine therapy will provide a useful diagnostic index to differentiate subjects with neuropathy-associated pain amenable to treatment using the methods and compositions of the invention (see, e.g., Crosby et al., J. Pain Symptom Manage. 19(1):35-9, 2000; Chen et al., J. Neurophysiol. 87:2726-2733, 2002; Shir et al., Harefuah 118(8):452-4, 1990, each incorporated herein by reference). Accordingly, in certain aspects of the invention the compositions and methods herein are directed toward treatment of a neuropathic disorder in individuals whose pain symptoms are insufficiently relieved by opioid treatment, and/or to treatment using other classes of analgesic drugs effective for treating nociceptive pain, such as NSAIDs. In this context, patients presenting with neuropathic disorders who will amenable for treatment using the compositions and methods of the invention will often show less than a 50% reduction in the severity or frequency of their pain symptoms following administration of a nociceptive pain therapeutic agent (e.g., an opiate or NSAID) compared to placebo-treated or other suitable control subjects. In certain cases, the subject patients will show less than a 30%, 20%, or 10% reduction, or no measurable reduction, in the severity or frequency of pain symptoms after receiving the nociceptive pain drug, compared to control subjects exhibiting similar pain symptoms.

In view of the distinct etiology of sensory symptoms associated with neuropthies, the activity and uses described herein for bicifadine and other 1-aryl-3-azabicyclo[3.1.0]hexanes would not have been predicted with a reasonable expectation of success by persons of ordinary skill in the art. The disclosure herein marks the first discovery and documentation that bicifadine and other 1-aryl-3-azabicyclo[3.1.0]hexanes are potent and effective in alleviating symptoms of neuropathic pain in animal models widely accepted by those skilled in the art as reasonably predictive for and correlative to efficacy of drugs and treatment methods in other mammals, including humans. In particular, the methods and compositions of the invention have been tested and demonstrated to be effective in the spinal nerve ligation (Chung) model (see, e.g., Bennett, G. J., Chung, J. M., Honore, M., and Seltzer, Z. “Models of Neuropathic Pain. In: Current Protocols in Neuroscience” (J. N. Crawley, C. R. Gerfen, M. A. Rogawski, D. R. Sibley, P. Skolnick, and S. Wray, eds.) pp. 9.14.1-9.14.16. John Wiley & Sons, New York (2003); Morrow, T. J. “Animal Models of Painful Diabetic Neuropathy: The STZ rat model.” In: Current Protocols in Neuroscience (J. N. Crawley, C. R. Gerfen, M. A. Rogawski, D. R. Sibley, P. Skolnick, and S. Wray, eds.) pp. 9.18.1-9.18.1 1. John Wiley & Sons, New York (2004), each incorporated herein by reference). The findings disclosed herein based on widely accepted models of neuropathic pain (i.e., the spinal nerve ligation model and STZ diabetes induced model), using well accepted endpoints modeling the symptoms associated with neuropathy, including thermal and mechanical-hyperalgesia, as described below, evince that the methods and compositions of the invention are effective for treating symptoms associated with neuropathies, including neuropathic pain, in mammalian subjects.

The methods and compositions of the invention for treating or preventing neuropathic disorders and/or related symptoms generally employ an effective amount of a 1-aryl-3-azabicyclo[3.1.0]hexane as described above, optionally formulated with one or more additional components, such as physiologically-compatible carriers, buffers, excipients, preservatives, and the like. As used herein, the term 1-aryl-3-azabicyclo[3.1.0]hexane includes all active and effective members of this group that are useful for treating or preventing a neuropathic disorder and/or related symptom(s), as exemplified by the diverse assemblages of compounds described herein, as well as all active derivatives, enantiomers, salts, polymorphs, solvates, hydrates, and/or prodrugs of these disclosed compounds. 1-Aryl-3-azabicyclo[3.1.0]hexanes selected for use within the therapeutic compositions and methods herein will be therapeutically effective and well tolerated among mammalian subjects, in useful and commercially feasible dosage amounts as indicated below, and without unacceptable adverse side effects. In more detailed embodiments, the compounds, compositions and methods of the invention are therapeutically effective to alleviate one or more neuropathic conditions and/or related symptoms identified herein, including any combination of these neuropathic conditions and/or related symptoms, without unacceptable adverse side effects. In certain embodiments, the therapeutic methods and compositions of the invention effectively treat and/or prevent a neuropathic condition or symptom, while avoiding or reducing one or more side effects associated with a current alternate drug treatment for neuropathy. In this context, the methods and compositions of the invention for treating a neuropathic disorder and/or related symptom(s) will often yield a reduction or elimination of one or more side effect(s) observed with alternate drug or non-drug treatments for neuropathies, including, but not limited to, sedation, respiratory impairment, sleep impairment, dizziness, loss of motor function, disorientation, memory loss or other cognitive impairment, mood disorders, constipation, dry mouth, low blood pressure, weight gain, eruption, dyspepsia, problems with cardiac function, dependence and/or withdrawal, among other side effects.

1-Aryl-3-azabicyclo[3.1.0]hexanes for use within the methods and compositions of the invention can be optionally formulated with a pharmaceutically acceptable carrier and/or various excipients, vehicles, stabilizers, buffers, preservatives, etc. Operable compounds within these aspects of the invention can be readily selected from among the various exemplary candidate compounds described herein using well-known methods, including the various animal models described below. These and other methods can be used to select, identify, and determine optimal dosages and combinations of the compounds described herein. Within the therapeutic methods and compositions of the invention, a 1-aryl-3-azabicyclo[3.1.0]hexane selected for use in a composition or method for treating or preventing a neuropathic disorder and/or related symptom(s) will be formulated for therapeutic use in an “effective amount,” “therapeutic amount,” or “effective dose”. These terms collectively describe either an effective amount or dose of a compound as described herein that is sufficient to elicit a desired pharmacological or therapeutic effect in a mammalian subject—typically resulting in a measurable reduction in an occurrence, frequency, or severity of a neuropathic disorder, and/or of one or more symptom(s) associated with a neuropathic disorder, in the subject. In certain embodiments, when a compound of the invention is administered to treat a neuropathic disorder, for example a neuropathic disorder characterized by one or more symptom(s) of neuropathic pain, an effective amount of the compound will be an amount sufficient in vivo to delay or eliminate onset of one or more symptoms associated with the neuropathic disorder, for example one or more neuropathic pain symptoms. Therapeutically-effective formulations and dosages can alternatively be determined by an administered formulation/dosage that yields a decrease in the occurrence, frequency or severity of one or more symptoms of a neuorpathic disorder, for example by a decline in the frequency or intensity of one or more neuropathic pain symptom(s). An effective amount of a 1-aryl-3-azabicyclo[3.1.0]hexane in this context will typically yield a detectable, therapeutic reduction in the nature or severity, occurrence, frequency, and/or duration of one or more symptom(s) associated with the targeted neuropathic condition or disorder. Therapeutically effective amounts, and dosage regimens, of the 1-aryl-3-azabicyclo[3.1.0]hexane compositions of the invention, including pharmaceutically effective salts, solvates, hydrates, polymorphs or prodrugs thereof, will be readily determinable by those of ordinary skill in the art, often based on routine clinical or patient-specific factors.

Alternatively, the efficacy of the methods and compositions of the invention for treating or preventing a neuropathic disorder and/or related symptom(s) can be demonstrated by various numerical evaluation and scale rating systems including, but not limited to, the neuropathic pain scale, the numeric rating scale, the visual analog scale, the faces pain scale, the brief pain inventory, the McGill pain questionnaire, or the initial pain assessment tool, all of which clinical rating systems are well known and widely accepted in the art for predicting clinical efficacy of neuropathic treatments. Using the neuropathic pain scale of 1 to 10, for example, effectiveness of the compounds and methods of the invention may be demonstrated by a decrease in a numerical value of a patient's assessment of pain over time in treatment. The decrease may be a decrease of at least one point on the scale to nine points on the scale, or a decrease of any value in between. Therapeutically effective amounts and dosage regimens of 1-aryl-3-azabicyclo[3.1.0]hexanes in these contexts will be readily determinable by those of ordinary skill in the art, often based on routine clinical or patient-specific factors.

Suitable routes of administration for the 1-aryl-3-azabicyclo[3.1.0]hexanes and related formulations of the invention to treat or prevent a neuropathic disorder and/or related symptom(s) include, but are not limited to, oral, buccal, nasal, aerosol, topical, transdermal, mucosal, injectable, slow release, controlled release, although various other known delivery routes, devices and methods can likewise be employed. Useful injectable delivery methods include, but are not limited to, intravenous, intramuscular, intraperitoneal, intraspinal, intrathecal, intracerebroventricular, intraarterial, and subcutaneous injection.

Suitable effective unit dosage amounts of 1-aryl-3-azabicyclo[3.1.0]hexanes for mammalian subjects may range from about 1 to 1200 mg, 50 to 1000 mg, 75 to 900 mg, 100 to 800 mg, or 150 to 600 mg. In certain embodiments, the effective unit dosage will be selected within narrower ranges of, for example, 10 to 25 mg, 30 to 50 mg, 75 to 100 mg, 100 to 150 mg, 150 to 250 mg or 250 to 500 mg. These and other effective unit dosage amounts may be administered in a single dose, or in the form of multiple daily, weekly or monthly doses, for example in a dosing regimen comprising from 1 to 5, or 2-3, doses administered per day, per week, or per month. In exemplary embodiments, dosages of 10 to 25 mg,30 to 50 mg, 75 to 100 mg, 100 to 200 (anticipated dosage strength) mg, or 250 to 500 mg, are administered one, two, three, or four times per day. In more detailed embodiments, dosages of 50-75 mg, 100-150 mg, 150-200 mg, 250-400 mg, or 400-600 mg are administered once, twice daily or three times daily. In alternate embodiments, dosages are calculated based on body weight, and may be administered, for example, in amounts from about 0.5 mg/kg to about 30 mg/kg per day, 1 mg/kg to about 15 mg/kg per day, 1 mg/kg to about 10 mg/kg per day, 2 mg/kg to about 20 mg/kg per day, 2 mg/kg to about 10 mg/kg per day or 3 mg/kg to about 15 mg/kg per day.

The amount, timing and mode of delivery of compositions of the invention comprising an effective amount of a 1-aryl-3-azabicyclo[3.1.0]hexane will be routinely adjusted on an individual basis, depending on such factors as weight, age, gender, and condition of the individual, symptom presentation pattern, whether the administration is prophylactic or therapeutic, and on the basis of other factors known to effect drug delivery, absorption, pharmacokinetics, including half-life, and efficacy. An effective dose or multi-dose treatment regimen for the compounds of the invention will ordinarily be selected to approximate a minimal dosing regimen that is necessary and sufficient to substantially prevent or alleviate one or more symptom(s) of a neuropathic disorder, for example one or more neuropathic pain symptom(s), in the subject, as described herein. Thus, following administration of a 1-aryl-3-azabicyclo[3.1.0]hexane according to the formulations and methods of the invention, test subjects will exhibit a 10%, 20%, 30%, 50% or greater reduction, up to a 75-90%, or 95% or greater, reduction, in one or more symptoms associated with the targeted neuropathy, compared to placebo-treated or other suitable control subjects.

Within additional aspects of the invention, combinatorial formulations and coordinate administration methods are provided which employ an effective amount of one or more 1-aryl-3-azabicyclo[3.1.0] hexanes, including pharmaceutically effective enantiomers, salts, solvates, hydrates, polymorphs or prodrugs thereof, and one or more additional active agent(s) that is/are combinatorially formulated or coordinately administered with the 1-aryl-3-azabicyclo[3.1.0] hexane(s)—yielding a combinatorial formulation or coordinate administration method that is effective to modulate, alleviate, treat or prevent one or more symptom(s) of a targeted neuropathic condition in a mammalian subject. Exemplary combinatorial formulations and coordinate treatment methods in this context employ a 1-aryl-3-azabicyclo[3.1.0]hexane in combination with one or more additional or adjunctive treatment agents or methods for treating neuropathy, for example one or more of the following neuropathy treatment agents and methods: NSAIDs, including but not limited to aspirin, ibuprofen, and COX-2 inhibitors, synthetic and natural opiates including but not limited to oxycodone, meperidine, morphine, and codeine; mexiletine; baclofen; tramadol; antiarrhythmics; anticonvulsants (e.g., lamotrigine, gabapentin, valproic acid, topiramate, famotodine, phenobarbital, diphenylhydantoin, phenytoin, mephenytoin, ethotoin, mephobarbital, primidone, carbamazepine, ethosuximide, methsuximide, phensuximide, trimethadione, benzodiazepines such as diazepam, phenacemide, acetazolamide, progabide, clonazepam, divalproex sodium, magnesium sulfate injection, metharbital, paramethadione, phenytoin sodium, valproate sodium, clobazam, sulthiame, dilantin, diphenylan and L-5-hydroxytryptophan); capsaicin cream; membrane-stabilizing drugs (e.g., lidocaine); N-methyl-D-aspartate receptor (NMDA) antagonists such as ketamine, surgery; transcutaneous electrical nerve stimulation; epidural spinal cord stimulation; neurectomy; rhizotomy; dorsal root entry zone lesion; cordotomy; thalamotomy; and neuroablation.

To practice a coordinate neuropathy treatment method of the invention, a 1-aryl-3-azabicyclo[3.1.0]hexane as described herein is administered, simultaneously or sequentially, in a coordinate treatment protocol with one or more of the secondary or adjunctive therapeutic agents or methods described above. The coordinate administration may be done simultaneously or sequentially in either order, and there may be a time period while only one or both (or all) active therapeutic agents, individually and/or collectively, exert their biological activities. A distinguishing aspect of all such coordinate treatment methods is that the 1-aryl-3-azabicyclo[3.1.0]hexane exerts at least some detectable therapeutic activity as described herein, and/or elicit a favorable clinical response, which may or may not be in conjunction with a secondary clinical response provided by the secondary therapeutic agent. Often, the coordinate administration of a 1-aryl-3-azabicyclo[3.1.0]hexane with a secondary therapeutic agent as contemplated herein will yield an enhanced therapeutic response beyond the therapeutic response elicited by either or both the 1-aryl-3-azabicyclo[3.1.0]hexane and/or secondary therapeutic agent alone.

Pharmaceutical dosage forms of 1-aryl-3-azabicyclo[3.1.0]hexanes within the instant invention may further include one or more excipients or additives, including, without limitation, binders, fillers, lubricants, emulsifiers, suspending agents, sweeteners, flavorings, preservatives, buffers, wetting agents, disintegrants, effervescent agents and other conventional excipients and additives. The compositions of the invention for treating neuropathic disorders can thus include any one or combination of the following: a pharmaceutically acceptable carrier or excipient; other medicinal agent(s); pharmaceutical agent(s); adjuvants; buffers; preservatives; diluents; and various other pharmaceutical additives and agents known to those skilled in the art. These additional formulation additives and agents will often be biologically inactive and can be administered to patients without causing deleterious side effects or interactions with the active agent.

If desired, the 1-aryl-3-azabicyclo[3.1.0]hexanes of the invention can be administered in a controlled release form by use of a slow release carrier, such as a hydrophilic, slow release polymer. Exemplary controlled release agents in this context include, but are not limited to, hydroxypropyl methyl cellulose, having a viscosity in the range of about 100 cps to about 100,000 cps.

1-Aryl-3-azabicyclo[3.1.0]hexanes and related compositions of the invention will often be formulated and administered in an oral dosage form, optionally in combination with a carrier or other additive(s). Suitable carriers common to pharmaceutical formulation technology include, but are not limited to, microcrystalline cellulose, lactose, sucrose, fructose, glucose dextrose, or other sugars, di-basic calcium phosphate, calcium sulfate, cellulose, methylcellulose, cellulose derivatives, kaolin, mannitol, lactitol, maltitol, xylitol, sorbitol, or other sugar alcohols, dry starch, dextrin, maltodextrin or other polysaccharides, inositol, or mixtures thereof. Exemplary unit oral dosage forms for use in this invention include tablets, which may be prepared by any conventional method of preparing pharmaceutical oral unit dosage forms can be utilized in preparing oral unit dosage forms. Oral unit dosage forms, such as tablets, may contain one or more conventional additional formulation ingredients, including, but not limited to, release modifying agents, glidants, compression aides, disintegrants, lubricants, binders, flavors, flavor enhancers, sweeteners and/or preservatives. Suitable lubricants include stearic acid, magnesium stearate, talc, calcium stearate, hydrogenated vegetable oils, sodium benzoate, leucine carbowax, magnesium lauryl sulfate, colloidal silicon dioxide and glyceryl monostearate. Suitable glidants include colloidal silica, fumed silicon dioxide, silica, talc, fumed silica, gypsum and glyceryl monostearate. Substances which may be used for coating include hydroxypropyl cellulose, titanium oxide, talc, sweeteners and colorants. The aforementioned effervescent agents and disintegrants are useful in the formulation of rapidly disintegrating tablets known to those skilled in the art. These typically disintegrate in the mouth in less than one minute, and often in less than thirty seconds. By effervescent agent is meant a couple, typically an organic acid and a carbonate or bicarbonate.

Additional compositions of the invention comprise a 1-aryl-3-azabicyclo[3.1.0]hexane prepared and administered in any of a variety of inhalation or nasal delivery forms known in the art. Devices capable of depositing aerosolized 1-aryl-3-azabicyclo[3.1.0]hexane formulations in the sinus cavity or pulmonary alveoli of a patient include metered dose inhalers, nebulizers, dry powder generators, sprayers, and the like. Methods and compositions suitable for pulmonary delivery of drugs for systemic effect are well known in the art. Suitable formulations, wherein the carrier is a liquid, for administration, as for example, a nasal spray or as nasal drops, may include aqueous or oily solutions of 1-aryl-3-azabicyclo[3.1.0]hexanes and any additional active or inactive ingredient(s).

Intranasal delivery permits the passage of such a compound to the blood stream directly after administering an effective amount of the compound to the nose, without requiring the product to be deposited in the lung. In addition, intranasal delivery can achieve direct, or enhanced, delivery of the active compound to the central nervous system. For intranasal and pulmonary administration, a liquid aerosol formulation will often contain a 1-aryl-3-azabicyclo[3.1.0]hexane as described herein combined with a dispersing agent and/or a physiologically acceptable diluent. Alternative, dry powder aerosol formulations may contain a finely divided solid form of the subject compound and a dispersing agent allowing for the ready dispersal of the dry powder particles. With either liquid or dry powder aerosol formulations, the formulation must be aerosolized into small, liquid or solid particles in order to ensure that the aerosolized dose reaches the mucous membranes of the nasal passages or the lung. The term “aerosol particle” is used herein to describe a liquid or solid particle suitable of a sufficiently small particle diameter, e.g., in a range of from about 2-5 microns, for nasal or pulmonary distribution to targeted mucous or alveolar membranes. Other considerations include the construction of the delivery device, additional components in the formulation, and particle characteristics. These aspects of nasal or pulmonary administration of drugs are well known in the art, and manipulation of formulations, aerosolization means, and construction of delivery devices, is within the level of ordinary skill in the art.

Yet additional compositions and methods of the invention are provided for topical administration of 1-aryl-3-azabicyclo[3.1.0]hexanes for the treatment of neuropathic disorders in mammals. Topical compositions may comprise a 1-aryl-3-azabicyclo[3.1.0]hexane and any other active or inactive component(s) incorporated in a dermatological or mucosal acceptable carrier, including in the form of aerosol sprays, powders, dermal patches, sticks, granules, creams, pastes, gels, lotions, syrups, ointments, impregnated sponges, cotton applicators, or as a solution or suspension in an aqueous liquid, non-aqueous liquid, oil-in-water emulsion, or water-in-oil liquid emulsion. These topical compositions may feature the 1-aryl-3-azabicyclo[3.1.0]hexane dissolved or dispersed in a portion of water or other solvent or liquid to be incorporated in the topical composition or delivery device. Transdermal administration may be enhanced by the addition of a dermal penetration enhancer known to those skilled in the art. Formulations suitable for such dosage forms incorporate excipients commonly utilized therein, particularly means, e.g. structure or matrix, for sustaining the absorption of drug over an extended period of time, for example 24 hours.

Yet additional formulations of 1-aryl-3-azabicyclo[3.1.0]hexanes for treating neuropathic disorders are provided for parenteral administration, including aqueous and non-aqueous sterile injection solutions which may optionally contain anti-oxidants, buffers, bacteriostats and/or solutes which render the formulation isotonic with the blood of the mammalian subject; and aqueous and non-aqueous sterile suspensions which may include suspending agents and/or thickening agents. The formulations may be presented in unit-dose or multi-dose containers. These and other formulations of the invention may also include polymers for extended release following parenteral administration. Extemporaneous injection solutions, emulsions and suspensions may be prepared from sterile powders, granules and tablets of the kind previously described. Exemplary unit dosage formulations are those containing a daily dose or unit, daily sub-dose, as described herein above, or an appropriate fraction thereof, of the active ingredient(s).

In other detailed embodiments, 1-aryl-3-azabicyclo[3.1.0]hexane compositions may be encapsulated for delivery in microcapsules, microparticles, or microspheres, prepared, for example, by coacervation techniques or by interfacial polymerization, for example, hydroxymethylcellulose or gelatin-microcapsules and poly(methylmethacrylate) microcapsules, respectively, in colloidal drug delivery systems (for example, liposomes, albumin microspheres, microemulsions, nano-particles and nanocapsules) or in macroemulsions.

The pharmaceutical agents and formulations of the current invention will typically be sterile or readily sterilizable, biologically inert, and easily administered.

The following examples illustrate certain embodiments of the present invention, and are not to be construed as limiting the present disclosure. The evidence provided in these examples demonstrates that 1-aryl-3-azabicyclo[3.1.0]hexanes as described herein are effective in the treatment of neuropathic disorders and related symptoms, including neuropathic pain, in mammals.

### EXAMPLES

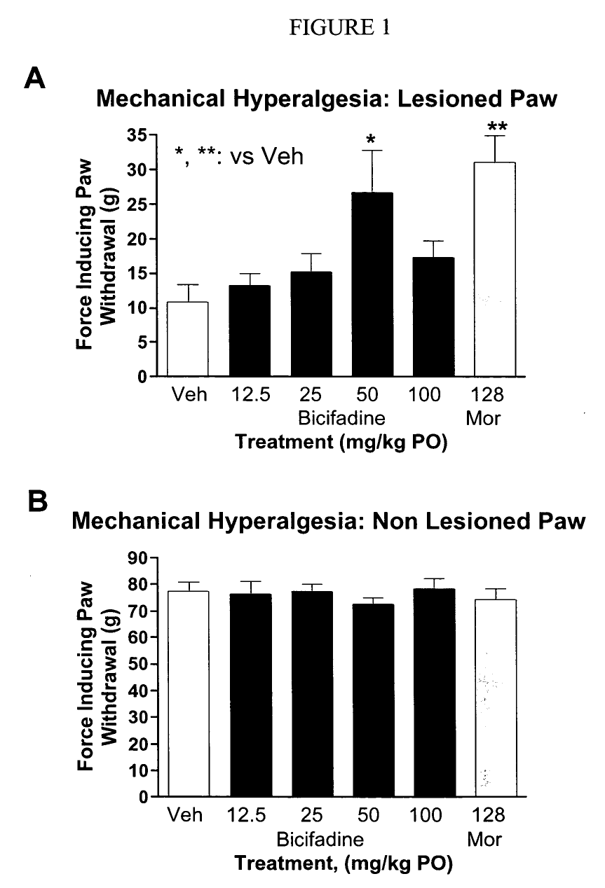

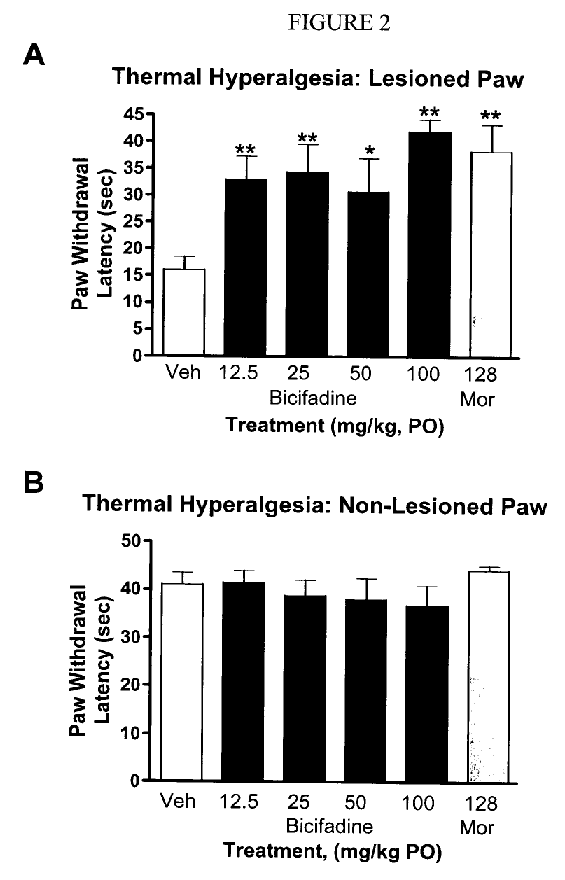

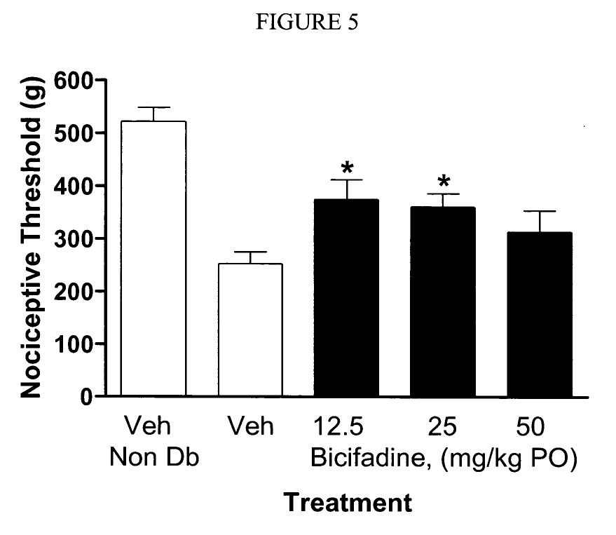

Bicifadine was tested in two art-accepted models of neuropathic pain—a model of neuropathic pain produced by spinal nerve ligation (Kim, et al., 1992) and the streptozotocin (STZ)-induced diabetic rat model (Morrow, T. J., 2004). In the spinal nerve ligation model (described in further detail in Example 1, below), rats were administered bicifadine or morphine orally three weeks after surgery. Sixty minutes later, mechanical hyperalgesia was measured based on the amount of force required to cause the rat to withdraw the lesioned paw (FIG. 1, Panel A), as compared to the non-lesioned paw (FIG. 1, Panel B). As can be seen in FIG. 1, Panel A bicifadine produced a statistically significant increase in the amount of pressure the rats were able to tolerate compared to vehicle. Further, bicifadine was more potent than morphine in this regard. In fact, the amount of morphine required to produce an effect equivalent to bicifadine in this model of neuropathic pain was a near lethal dose. The ability of bicifadine to alleviate the symptoms associated with a neuropathic disorder is further demonstrated in FIG. 2, which depicts thermal hyperalgesic response based on the sensitivity of rats to application of a thermal stimulus to a lesioned (FIG. 2, Panel A), compared to an non-lesioned (FIG. 2, Panel B) paw. The observation that bicifadine does not affect either the pressure threshold or response to application of heat in the non-lesioned paw (FIG. 1, Panel B; FIG. 2, Panel B) indicates that the analgesia produced by bicifadine as evidenced in the Chung model is not due to debilitation of the animal by the drug. Bicifadine also increased the amount of pressure an animal could withstand on a lesioned paw in the STZ diabetic neuropathy model (see, Example 3, below; FIG. 5). In this figure, the increased sensitivity of the animal to mechanical pressure is illustrated with a decrease in the amount of force applied eliciting a withdrawal compared to control (non STZ treated) rats. Bicifadine is able to restore the amount of pressure tolerated in these diabetic rats to values approaching those obtained in control animals (FIG. 5).

### Example 1

### Bicifadine Effectively Reduces Tactile Hyperalgesia and Thermal Hyperalgesia in the Spinal Nerve Ligation Model

Tight ligature of spinal nerves in rats is associated with hyperalgesia, allodynia and spontaneous pain, and thereby provides an accepted model for peripheral neuropathic pain in humans. Male Sprague-Dawley rats Rj:SD (IOPS Han) weighing 218-260 g at the beginning of the procedure were anesthetized (sodium pentobarbital 40 mg/kg i.p.) and an incision at the L4-S2 level was performed to expose the left L5 and L6 spinal nerves, which were then tightly ligated (4-0 silk suture) distal to the dorsal root ganglion and prior to entrance into the sciatic nerve, as first described by Kim and Chung (Pain 50:355-363, 1992). The wound was then sutured, and the rats received an i.m. injection of clamoxyl (100 mg/kg s.c.) and were allowed to recover. 4 weeks after surgery, when the chronic pain state was fully installed, rats were submitted consecutively to tactile and thermal stimulation of both the non-lesioned and the lesioned hindpaws.

The foregoing procedure results in mechanical (tactile) allodynia in the left hind paw as assessed by recording the pressure at which the affected paw (ipsilateral to the site of nerve injury) was withdrawn from graded stimuli (von Frey filaments ranging from 4.0 to 148.1 mN) applied perpendicularly to the plantar surface of the paw (between the footpads) through wire-mesh observation cages. A paw withdrawal threshold (PWT) was determined by sequentially increasing and decreasing the stimulus strength and analyzing withdrawal data using a Dixon non-parametric test, as described by Chaplan et al., 1994.

Normal rats and sham surgery rats (nerves isolated but not ligated) withstand at least 148.1 mN (equivalent to 15 g) of pressure without responding. Spinal nerve ligated rats respond to as little as 4.0 mN (equivalent to 0.41 g) of pressure on the affected paw. Rats are included in the study only if they do not exhibit motor dysfunction (e.g., paw dragging or dropping) and their PWT was below 39.2 mN (equivalent to 4.0 g). Three weeks after surgery rats are treated with test compounds or control diluent (PBS) once by s.c. injection and PWT is determined each day thereafter for 7 days. The observed PWT values for rats treated with an effective amount of a 1-aryl-3-azabicyclo[3.1.0]hexane test compounds described herein will be measurably increased compared to PWT values observed for control animals.

For tactile stimulation, the animal was placed under an inverted acrylic plastic box (18×11.5×13 cm) on a grid floor. The tip of an electronic Von Frey probe (Bioseb, model 1610) was then applied with increasing force to the non-lesioned and lesioned hindpaws and the force inducing paw-withdrawal was automatically recorded. This procedure was carried out 3 times and the mean force per paw was calculated.

For thermal stimulation, the apparatus (Model 7200, Ugo Basile, Italy) consists of individual acrylic plastic boxes (17×11×13 cm) placed upon an elevated glass floor. A rat was placed in the box and left free to habituate for 10 minutes. A mobile infrared radiant source (96±10 mW/cm²) was then focused under the non-lesioned and lesioned hindpaws and the paw-withdrawal latency was automatically recorded. In order to prevent tissue damage the heat source was automatically turned off after 45 seconds.

Behavioral testing was carried out 2 weeks after surgery. Prior to receiving drug treatment all animals were submitted to tactile stimulation and assigned to treatment groups matched on the basis of their pain response. 8 rats were studied per group, and the tests were performed blind.

Bicifadine was evaluated at the indicated doses, administered p.o. 60 minutes before the test, and compared with a vehicle control group. Morphine (128 mg/kg p.o.), used as a reference substance, was administered under the same experimental conditions.

Data were analyzed by comparing the responses of lesioned paws in the treatment groups with vehicle control group using paired and unpaired Student's t tests.

As demonstrated in FIGS. 1 and 2, bicifadine effectively suppressed mechanical and thermal hyperalgesia in the Chung model of chronic neuropathic pain. Vehicle treated rats (open bars) showed a significant reduction in the threshold for withdrawl of the paw on the lesioned side following the application of mechanical pressure (FIG. 1, Panel A) or thermal stimulus (FIG. 2, Panel A). Side-by-side comparisons in these figures show that morphine, at a nearly lethal dose, caused a significant increase in the threshold for nociceptive response (hatched bars). As shown in the figures, 50 mg/kg PO bicifadine resulted in a significant increase in the force required to induce paw withdrawal compared to vehicle treated, lesioned paws. In addition, all doses of bicifadine tested (12.5-100 mg/kg PO) resulted in a significant increase in paw withdrawal latency in response to a thermal stimulus. The magnitude of this effect was approximately equivalent to that of a nearly lethal dose of morphine, 128 mg/kg PO.

### Example 2

### 1-(3,4-dichlorophenyl)-3-azabicyclo[3.1.0]hexane Effectively Reduces Tactile Hyperalgesia and Thermal Hyperalgesia in the Spinal Nerve Ligation Model

In the instant example, another exemplary, substituted 1-aryl-3-azabicyclo[3.1.0]hexane was evaluated for its ability to alleviate symptoms associated with neuropathy in the spinal nerve ligation (Chung) model of neuropathic pain. In this study, a multiply aryl-subsituted compound as described above, 1-(3,4-dichlorophenyl)-3-azabicyclo[3.1.0]hexane was assayed according to the procedures described above in Example 1. The 1-(3,4-dichlorophenyl)-3-azabicyclo[3.1.0]hexane was administered to test animals at the indicated doses p.o. 60 minutes before the test, and compared with a vehicle control group. Data were analyzed by comparing the responses of lesioned paws in the treatment groups with vehicle control group using paired and unpaired Student's t tests, as described in Example 1.

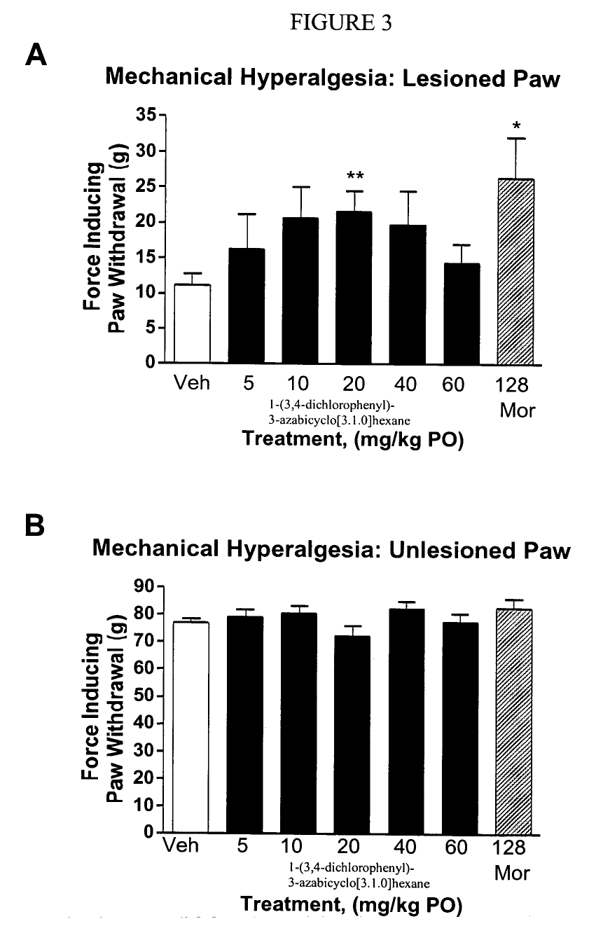

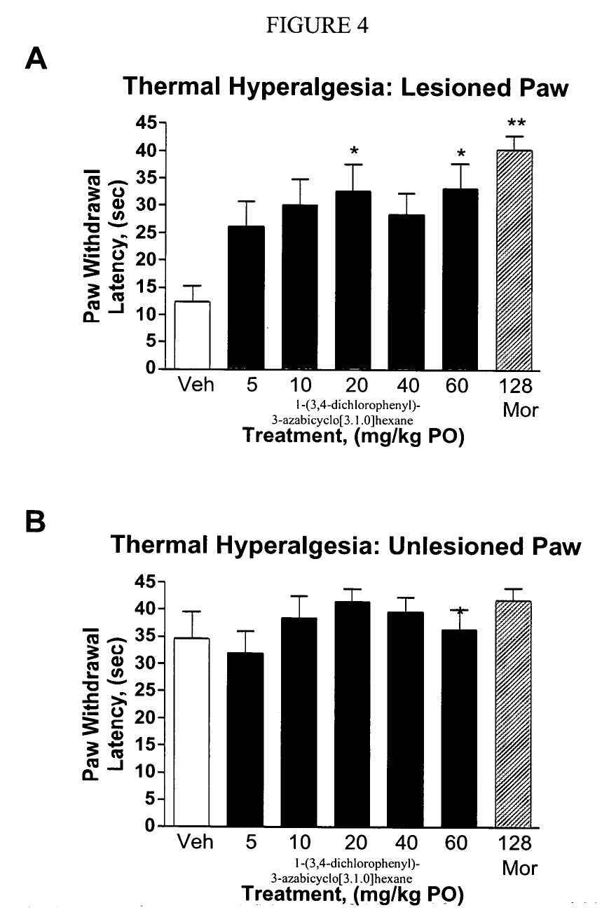

As demonstrated in FIGS. 3 and 4, respectively, 1-(3,4-dichlorophenyl)-3-azabicyclo[3.1.0]hexane potently suppressed tactile allodynia in the Chung model of neuropathic pain, and dose-dependently suppressed thermal hyperalgesia in the test subjects. Vehicle treated rats (open bars) showed a significant reduction in the threshold for withdrawal of the paw on the lesioned side following the application of mechanical pressure (FIG. 3, Panel A) or thermal stimulus (FIG. 4, Panel A). Administration of 1-(3,4-dichlorophenyl)-3-azabicyclo[3.1.0]hexane to the subjects resulted in a significant increase in the force required to induce paw withdrawal compared to vehicle treated, lesioned paws, as well as a significant increase in paw withdrawal latency in response to a thermal stimulus. FIG. 3, Panel B, and FIG. 4, Panel B (responses on the unlesioned side) demonstrate that these effects are not due to debilitation of the animal.

### Example 3

### Bicifadine Effectively Alleviates Neuropathy Symptoms in the Streptozotocin-Induced Diabetes Rat Model

Male Sprague Dawley rats Rj:SD (IOPS Han) weighing 218-260 g at the beginning of the experiment, were included in this study for the induction of diabetes. These were divided into vehicle control and streptozotocin treated groups. The rats were housed in a temperature (19.5-24.5° C.) and relative humidity (45-65%) controlled room with a 12-h light/dark cycle, with ad libitum access to filtered tap water and standard pelleted laboratory chow throughout the study.

On day 0, diabetes was induced by intraperitoneal injection of streptozotocin (STZ; 75 mg/kg) to the rats. On day 23, to confirm the presence of diabetes in the STZ-administered rats, hyperglycemia was measured using blood glucose strips. Animals with a blood sugar level lower than 250 mg/l were not used in further investigations.

Animals found useful for inclusion in additional investigations were dosed orally with bicifadine at the indicated doses, and the nociceptive threshold tested sixty minutes later. The nociceptive threshold was evaluated using a mechanical nociceptive stimulus (paw pressure test). An increasing pressure was applied onto the hindpaw of the animal until the nociceptive response (vocalization or paw withdrawal) was reached. The pain threshold (grams of contact pressure) was measured in both hindpaws.

Results are expressed as the nociceptive threshold (mean±SEM) in grams of contact pressure for each group, calculated from individual nociceptive thresholds (mean values of the nociceptive thresholds obtained for both hindpaws). Statistical significance between the bicifadine treated groups and the vehicle-treated diabetic group was determined by a Kruskal-Wallis test, using the residual variance after a one-way analysis of variance (P<0.05).

As demonstrated by the data provided in FIG. 5, bicifadine reduces mechanical hyperalgesia in rats with diabetic neuropathy. Twenty three days after diabetes was induced in rats with STZ, rats with significant manifestations of diabetes were orally administered either vehicle or bicifadine, and their nociceptive threshold determined 60 minutes later. Rats treated with vehicle showed a significant reduction in the paw pressure required to elicit a nociceptive response (paw withdrawal or squeak). In contrast, diabetic rats treated with 12.5 and 25 mg/kg bicifadine (closed bars) showed a significant (P<0.05) increase in the nociceptive threshold relative to the vehicle treated diabetic animals (open bars).

### Example 4

### Chronic Constriction Injury Model

Another useful model for demonstrating efficacy of the methods and compositions of the invention for treating a neuropathic disorder and/or related symptom(s) is the chronic constriction injury model. In this model, a unilateral peripheral hyperalgesia is produced in rats by nerve ligation (Bennett, et al., Pain, 33:87-107, 1988). Sprague-Dawley rats (250-350 g) are anesthetized with sodium pentobarbital and the common sciatic nerve is exposed at the level of the mid thigh by blunt dissection through the biceps femoris. A section of nerve (about 7 mm), proximal to the sciatic trifucation, is freed of tissue and ligated at four positions with chromic gut suture, with the suture tied with about 1 mm spacing between ligatures. The incision is closed in layers and the animals allowed to recuperate. Thermal hyperalgesia is measured using a paw-withdrawal test (Hargreaves, et al., Pain, 32:77-88, 1988). To perform the test, animals are habituated on an elevated glass floor and a radiant heat source aimed at the mid-plantar hindpaw (sciatic nerve territory) through the glass floor with a 20 second cut-off to prevent injury to the skin. The latencies for the withdrawal reflex in both hind paws are recorded.

Paws with ligated nerves show shorter paw withdrawal latencies compared to the unoperated or sham operated paws. Responses to test compounds are evaluated at different times after oral administration to determine the onset and duration of compound effect. When performing the test, groups of rats receive either vehicle or the test compound orally three times daily for 5 days. Paw withdrawal latencies can be measured each day 10 min. before and two or three hr. after the first daily dose. Compound efficacy is calculated as mean percentage decrease of hyperalgesia compared to a vehicle-treated group. Compound potencies may be expressed as the minimum effective dose (MED) in mg/kg/day that yields a % decrease in hyperalgesia that is statistically significant, where the % anti-hyperalgesic effect may be calculated as follows: 2 (Mean of vehicle group−Mean of compound group) (Mean of vehicle group)×100. Animals treated with active, 1-aryl-3-azabicyclo[3.1.0]hexane compounds described herein will exhibit detectable decreases in hyperalgesia compared to control animals.

### Example 5

### Partial Sciatic Nerve Model of Induced Neuropathy

Another useful model for demonstrating efficacy of the methods and compositions of the invention for treating a neuropathic disorder and/or related symptom(s) is the partial sciatic nerve ligation model of neuropathic pain, which produces neuropathic hyperalgesia in rats (Seltzer et al., Pain, 43:205-218, 1990). Partial ligation of the left sciatic nerve is performed under enflurane/O₂ inhalation anesthesia. Following induction of anesthesia, the left thigh of the rat is shaved and the sciatic nerve exposed at high thigh level through a small incision and is carefully cleared of surrounding connective tissues at a site near the trocanther just distal to the point at which the posterior biceps semitendinosus nerve branches off of the common sciatic nerve. A 7-0 silk suture is inserted into the nerve with a ⅜ curved, reversed-cutting mini-needle and tightly ligated so that the dorsal ⅓ to ½ of the nerve thickness is held within the ligature. The wound is closed with a single muscle suture (7-0 silk) and a Michelle clip. Following surgery, the wound area is dusted with antibiotic powder. Sham-treated rats undergo an identical surgical procedure except that the sciatic nerve is not manipulated. Following surgery, animals are weighed and placed on a warm pad until they recover from anesthesia. Animals are then returned to their home cages until behavioral testing begins.

Responses to test compounds are evaluated at different times after oral administration to determine the onset and duration of compound effect. When performing the test, groups of rats would receive either vehicle or the test compound orally three times daily for 5 days. Paw withdrawal latencies can be measured each day 10 min. before and two or three hr. after the first daily dose.

The animal is assessed for response to noxious mechanical stimuli by determining hind paw withdrawal thresholds to a noxious mechanical stimulus using an analgesymeter (Model 7200, Ugo Basile, Italy), as described by Stein, 1988. The maximum weight that can be applied to the hind paw is set at 250 g and the end point is taken as complete withdrawal of the paw.

### Example 6

### Behavioral Testing for Mechanical Allodynia

Other useful models for demonstrating efficacy of the methods and compositions of the invention for treating a neuropathic disorder and/or related symptom(s) employ alternative protocols for behavioral assessment of allodynia. Test animals, such as those described in the examples above, may also be tested for sensitivity to non-noxious mechanical stimuli by determining the hindpaw withdrawal response to von Frey hair stimulation of the plantar surface of the footpad (Igarashi et al., Spine 25:2975-80, 2000). Rats are acclimated to being on a suspended 6-mm wire grid and having the plantar surface of their footpads stimulated with von Frey filaments. Three days prior to surgery, animals are habituated to acclimate the animals to movements and foot poking.

Responses to test compounds are evaluated at different times after oral administration to determine the onset and duration of compound effect. When performing the test, groups of rats would receive either vehicle or the test compound orally three times daily for 5 days. Paw withdrawal latencies can be measured each day 10 min. before and two or three hr. after the first daily dose.

Paw withdrawal latencies are measured using filaments. The filaments are calibrated so that between 1-15 g force are applied to the paw surface just until the filament bends, for a total of two applications approximately 2 to 3 seconds apart and varied in location so as to avoid sensitization. If the rat does not withdraw its foot after either of the two applications of a given filament, the next stiffer filament is tested in the same manner. When the rat withdraws its foot, the measurement is verified by ensuring that there is an absence of response at the next less stiff filament. The gram force of the filament causing the positive response is recorded for first reaction. After 5 minutes the same procedure is performed again. Baseline testing is performed three days prior to the start of the experiment to accommodate the animals to the testing procedure and to verify that they have normal responses. If the rat withdrew its foot, this gram force is recorded as a second reaction. A positive responder is identified as an animal responding to a filament gram force of less than 5 grams. Animals treated with the test compound show a decreasing sensitivity to the pressure, approaching a normal reaction.

### Example 7

### Pin Prick Test

Another useful model for demonstrating efficacy of the methods and compositions of the invention for treating a neuropathic disorder and/or related symptom(s) is known as the pin prick test. In this model, rats are confined within a clear plastic cage set on an elevated wire mesh floor with holes ˜1 cm in diameter. The tip of a safety pin is pressed against the skin of the plantar heel such that the skin is dimpled but not penetrated. The normal response to pin-prick is a nocifensive withdrawal reflex of very small amplitude and short duration. Following nerve injury, the response is greatly increased in amplitude and duration and the animal will frequently lick the stimulated site.

Responses to test compounds are evaluated at different times after oral administration to determine the onset and duration of compound effect. When performing the test, groups of rats would receive either vehicle or the test compound orally three times daily for 5 days. Reactions can be measured each day 10 min. before and two or three hr. after the first daily dose.

A decrease in amplitude and/or duration of the withdrawal indicates the effectiveness of the test compound.

### Example 8

### Cold Allodynia Test

Another useful model for demonstrating efficacy of the methods and compositions of the invention for treating a neuropathic disorder and/or related symptom(s) is know as the cold allodynia test. In one such method (Bennett, et al., Pain 33:87-107, 1988), rats are placed for 20 minutes on a metal plate cooled to 4° C. by water circulating beneath it. The number and duration of the nocifensive withdrawal reflexes that occur when the animal's symptomatic paw touches the floor are measured. These values can be compared to those obtained with the metal floor warmed to 30° C.

The effectiveness of test compounds can be determined by evaluation at different times after oral administration to determine the onset and duration of compound effect. When performing the test, groups of rats would receive either vehicle or the test compound orally three times daily for 5 days. Reactions can be measured each day 10 min. before and two or three hr. after the first daily dose.

An increase in the amount of time prior to withdrawal and/or decrease in the duration of withdrawal of the symptomatic paw indicates effectiveness of the test compound.

Although the foregoing invention has been described in detail by way of example for purposes of clarity of understanding, persons of ordinary skill in the art will understand that certain changes and modifications may be practiced within the scope of the appended claims which are presented by way of illustration not limitation. In this context, the invention is not limited to the particular formulations, processes, and materials disclosed herein, as such formulations, process steps, and materials may vary somewhat. Also, the terminology employed herein is used for describing particular embodiments only, and is not intended to be limiting of the invention embodied in the claims. Various publications and other reference information have been cited within the foregoing disclosure for economy of description. Each of these references is incorporated herein by reference in its entirety for all purposes. It is noted, however, that the various publications discussed herein are incorporated solely for their disclosure prior to the filing date of the present application, and the inventors reserve the right to antedate such disclosure by virtue of prior invention.

### REFERENCES

Armarego et al., J. Chem. Soc. [Section C: Organic] 19:3222-9, 1971.
Bennett, G. J. and Xie, Y. K. “A peripheral mononeuropathy in rat that produces disorders of pain sensation like those seen in man.” In: Pain, 33:87-107, 1988.
Bennett, G. J., Chung, J. M., Honore, M., and Seltzer, Z. “Models of Neuropathic Pain. In: Current Protocols in Neuroscience” (J. N. Crawley, C. R. Gerfen, M. A. Rogawski, D. R. Sibley, P. Skolnick, and S. Wray, eds.) pp. 9.14.1-9.14.16. John Wiley & Sons, New York (2003).
Cabadio et al., Fr. Bollettino Chimico Farmaceutico 117:331-42, 1978.
Chaplan, S. R.; Bach, F. W.; Pogrel, J. W.; Chung, J. M.; Yaksh, T. L. “Quantitative assessment of tactile allodynia in the rat paw.” In: J. Neurosci. Meth. 53:55-63 (1994).
Chen et al., J. Neurophysiol. 87:2726-2733, 2002.
Crosby et al., J. Pain Symptom Manage. 19(1):35-9, 2000.
Czobor P., et al., Stark J., Beer G., Petti S., Lippa A., Brown J., Beer B. “A Double-Blind, Placebo Controlled Randomized Study of DOV220,075 (bicifadine) SR and Codeine 60 mg in the Treatment of Post-Operative Dental Pain.” Presented at the 2nd Annual Scientific Meeting Mar. 20-23, 2003 Chicago, Ill. American Pain Society Abstract Database at http://www.ampainsoc.org/abstract/2003/data/index.html. (Poster #915));
Czobor P., Stark J., Beer G., Brown J., Sunshine A., Konery S., Turpin M., Olson N., Otero A., Lippa A., Beer B. “A two center double-blind, placebo-controlled randomized study of DOV 220,075 (bicifadine) SR and Tramadol 100 mg in the treatment of post-operative dental pain.” In: Pain, 2004: 5(1), Supplement 1, p 59. Presented at the Joint APS and Canadian Pain Society Annual Meeting (23rd APS Annual Scientific Meeting) May 6-9, 2004, Vancouver, BC Canada. American Pain Society Abstract Database at http://www.ampainsoc.org/abstract/2004/data/index.html (Poster #801)
Epstein et al., NIDA Res. Monogr. pp. 93-98, 1982.
Epstein et al., J. Med. Chem. 24 (5):481, 1981.
Hargreaves, K., Dubner, R., Brown, F., Flores, C., and Joris, J. “A new and sensitive method for measuring thermal nociception in cutaneous hyperalgesia.” In: Pain 32:77-88 (1988).
Igarashi et al., Spine, 25:2975-80 (2000).
Kim, S. H.; Chung, J. M. “An experimental model for peripheral neuropathy produced by segmental spinal nerve ligation in the rat.” In: Pain, 50:355-363, (1992); and “The streptozotocin (STZ)-induced diabetic rat model” (see, e.g., Morrow, T. J., 2004.) “Animal Models of Painful Diabetic Neuropathy: The STZ rat model.” In: Current Protocols in Neuroscience (J. N. Crawley, C. R. Gerfen, M. A. Rogawski, D. R. Sibley, P. Skolnick, and S. Wray, eds.) pp. 9.18.1-9.18.11. John Wiley & Sons, New York.
Marazzo et al., Arkivoc v: 156-169, 2004.
Max, M. B., Schafer, S. C., Culnane M., et al., “Association of pain relief with drug side effects in postherpetic neuralgia: a single dose study of clonidine, codeine, ibuprofen, and placebo.” In: Clinical and Pharmacolopgical Therapy, 43:363 (1988).
Max, M. B., Schafer, S. C., Culnane, M., et al., “Amitriptyline, but not lorazepam, relieves postherpetic neuralgia.” In: Neurology, 38:1427 (1988).
Morrow, T. J. “Animal Models of Painful Diabetic Neuropathy: The STZ rat model.” In: Current Protocols in Neuroscience (J. N. Crawley, C. R. Gerfen, M. A. Rogawski, D. R. Sibley, P. Skolnick, and S. Wray, eds.) pp. 9.18.1-9.18.11. John Wiley & Sons, New York (2004).
Mouzin et al., Synthesis 4:304-305, 1978.
“Nitrogen Protecting Groups in Organic Synthesis”, John Wiley and sons, New York, N.Y., 1981, Chapter 7; “Nitrogen Protecting Groups in Organic Chemistry”, Plenum Press, New York, N.Y., 1973, Chapter 2; See also, T. W. Green and P. G. M. Wuts in “Protective Groups in Organic Chemistry, 3rd edition” John Wiley & Sons, Inc. New York, N.Y., 1999.
Seltzer, Z., Dubner, R., and Shir, Y. “A Novel Behavioral Model of Neuropathic Pain Disorders Produced in Rats by Partial Sciatic Nerve Injury.” In: Pain, 43:205-218 (1990).
Shir et al., Harefuah 118(8):452-4, 1990.
Stein, C., “Unilateral Inflammation of the Hindpaw in Rats as a Model of Prolonged Noxious Stimulation: Alterations in Behavior and Nociceptive Thresholds.” In: Pharmacol. Biochem. Beh. 31:451-455, 1988.
Vilsmaier et al., Tetrahedron 45:3683-3694, 1989
Woolf, C. J., Mannion, R. J. “Neuropathic Pain: Aetiology, Symptoms, Mechanisms, and Management.” In: The Lancet, 353 (9168):1959-1964 (1999)
U.S. Provisional Patent Application No. 60/424,982
U.S. Patent Application No. 10/466,457
U.S. Patent Application No. 10/702,397
U.S. Patent Application No. 10/764,371
U.S. Patent Application No. 10/764,373
U.S. Patent Application No. 10/764,375
U.S. Patent Application No. 10/920,748
U.S. Pat. No. 4,118,417
U.S. Pat. No. 4,196,120
U.S. Pat. No. 4,435,419
U.S. Pat. No. 4,131,611
U.S. Pat. No. 4,231,935
U.S. Pat. No. 6,204,284
U.S. Pat. No. 6,372,919
U.S. Pat. No. 6,659,887
U.S. Pat. No. 6,716,868
PCT/US2003/035099 (International Publication No.W004/043920)
Patent publication PL 120095 B2, CAN 99:158251 by Szalacke et al

We claim:

1. A method for preventing or treating a neuropathic disorder in a mammalian subject comprising administering to said subject an effective amount of a compound of formula I 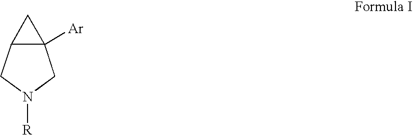
wherein Ar is a phenyl or other aromatic group having at least one substitution on the aryl ring, and wherein R is selected from hydrogen, C₁₋₆ alkyl, halo(C₁₋₆)alkyl, C₃₋₉ cycloalkyl, C₁₋₅ alkoxy(C₁₋₆)alkyl, carboxy(C₁₋₃)alkyl, C₁₋₃ alkanoyl, carbamate, halo(C₁₋₃)alkoxy(C₁₋₆)alkyl, C₁₋₃ alkylamino(C₁₋₆)alkyl, and di(C₁₋₃)alkylamino(C₁₋₆)alkyl, cyano(C₁₋₆)alkyl, methyl, ethyl, trifluoromethyl, trifluoroethyl and 2-methoxyethyl.

2. The method of claim 1, wherein the compound is selected from bicifadine, enantiomers of bicifadine, salts of bicifadine, prodrugs of bicifadine, polymorphs, hydrates, and solvates of bicifadine, and combinations thereof.

3. The method of claim 2, wherein the compound is bicifadine HCl.

4. The method of claim 2, wherein the compound comprises a (+) enantiomer of bicifadine.

5. The method of claim 4, wherein the compound is administered in a formulation that is substantially free of a (−) enantiomer of bicifadine.

6. The method of claim 2, wherein the compound comprises a (−) enantiomer of bicifadine.

7. The method of claim 6, wherein the compound is administered in a formulation that is substantially free of a (+) enantiomer of bicifadine.

8. The method of claim 2, wherein the compound comprises a polymorph B form of bicifadine.

9. The method of claim 8, wherein the compound is administered in a formulation that is substantially free of a polymorph A form of bicifadine.

10. The method of claim 1, wherein the compound is selected from the group consisting of: 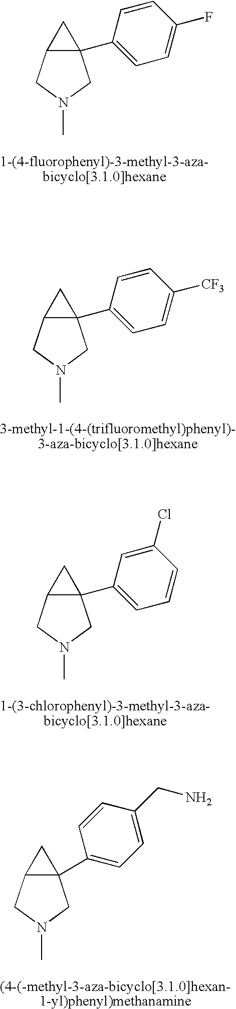
and pharmaceutically acceptable, active salts, solvates, hydrates, polymorphs, enantiomers, and prodrugs thereof.

11. The method of claim 1, wherein the compound is selected from the group consisting of: 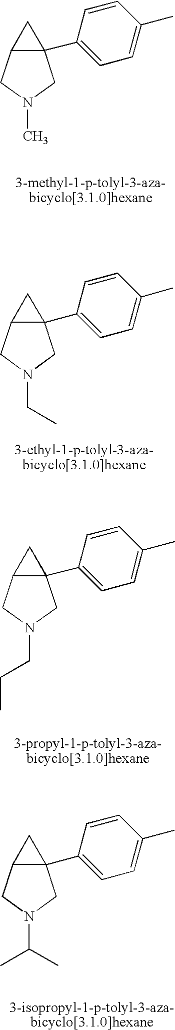 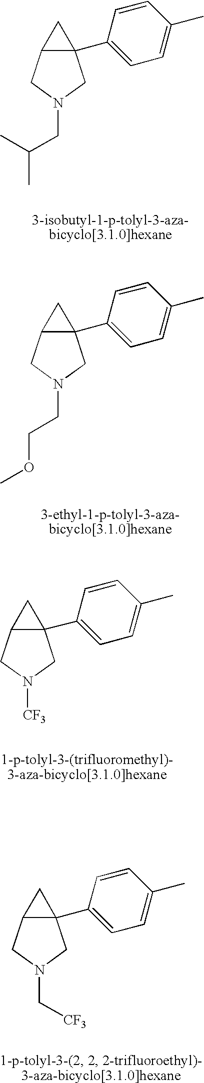
and pharmaceutically acceptable, active salts, solvates, hydrates, polymorphs, enantiomers, and prodrugs thereof.

12. A method for preventing or treating a neuropathic disorder in a mammalian subject comprising administering to said subject an effective amount of a compound of formula III 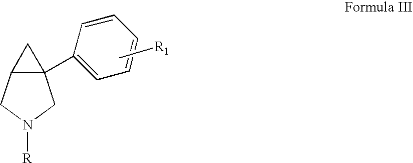
wherein R is selected from C₁₋₆ alkyl, halo(C₁₋₆)alkyl, C₃₋₉ cycloalkyl, C₁₋₅ alkoxy(C₁₋₆)alkyl, carboxy(C₁₋₃)alkyl, C₁₋₃ alkanoyl, carbamate, halo(C₁₋₃)alkoxy(C₁₋₆)alkyl, C₁₋₃ alkylamino(C₁₋₆)alkyl, and di(C₁₋₃)alkylamino(C₁₋₆)alkyl, cyano(C₁₋₆)alkyl, methyl, ethyl, trifluoromethyl, trifluoroethyl and 2-methoxyethyl; and
wherein R₁ is selected from halogen, C₁₋₃ alkyl, C₂₋₄ alkenyl, C₂₋₄ alkynyl, halo(C₁₋₃)alkyl, cyano, hydroxy, C₃₋₅ cycloalkyl, C₁₋₃ alkoxy, C₁₋₃ alkoxy(C₁₋₃)alkyl, carboxy(C₁₋₃)alkyl, C₁₋₃ alkanoyl, halo(C₁₋₃)alkoxy, nitro, amino, C₁₋₃ alkylamino, and di(C₁₋₃)alkylamino, methyl, ethyl, fluoro, chloro, trifluoromethyl, cyano, nitro, and trifluoromethoxy.

13. The method of claim 12, wherein the compound is selected from the group consisting of: 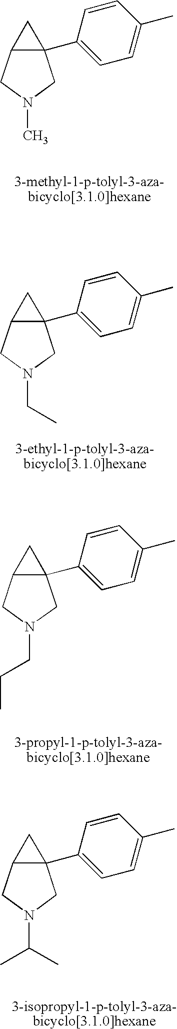 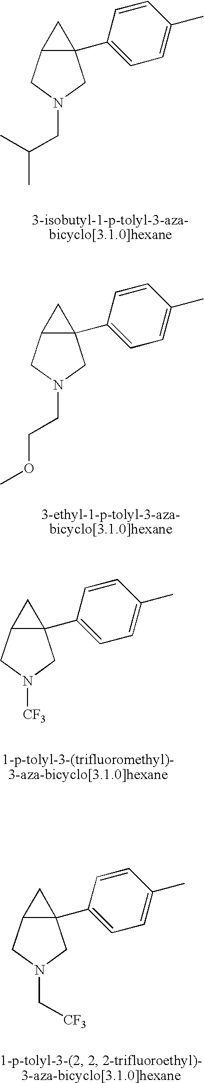
and pharmaceutically acceptable, active salts, solvates, hydrates, polymorphs, enantiomers, and prodrugs thereof.

14. A method for preventing or treating a neuropathic disorder in a mammalian subject comprising administering to said subject an effective amount of a compound of formula IV 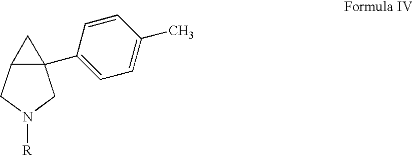
wherein R is selected from C₁₋₆ alkyl, halo(C₁₋₆)alkyl, C₃₋₉ cycloalkyl, C₁₋₅ alkoxy(C₁₋₆)alkyl, carboxy(C₁₋₃)alkyl, C₁₋₃ alkanoyl, carbamate, halo(C₁₋₃)alkoxy(C₁₋₆)alkyl, C₁₋₃ alkylamino(C₁₋₆)alkyl, and di(C₁₋₃)alkylamino(C₁₋₆)alkyl, cyano(C₁₋₆)alkyl, methyl, ethyl, trifluoromethyl, trifluoroethyl and 2-methoxyethyl.

15. The method of claim 14, wherein the compound is selected from: 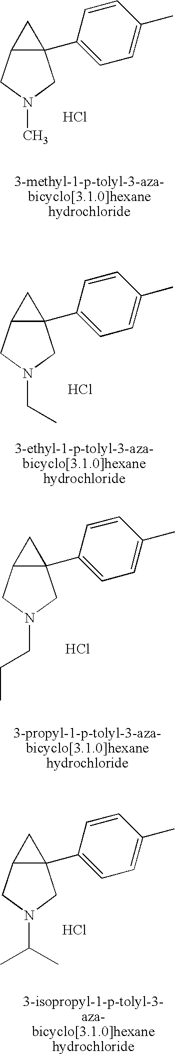 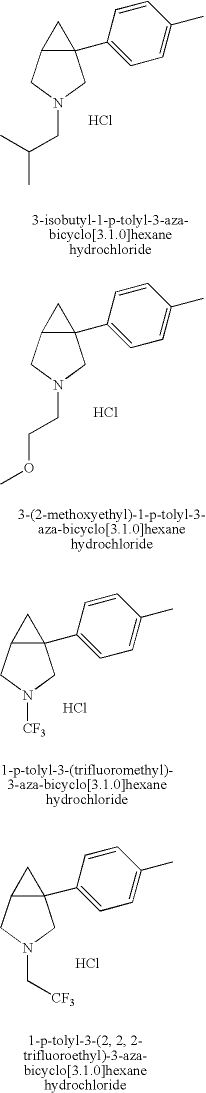
and pharmaceutically acceptable, active salts, solvates, hydrates, polymorphs, enantiomers, and prodrugs thereof.

16. A method for preventing or treating one or more symptom(s) resulting from a neuropathic disorder in a mammalian subject comprising administering to said subject an effective amount of a 1-aryl-3-azabicyclo[3.1.0]hexane.

17. The method of claim 16, wherein the 1-aryl-3-azabicyclo[3.1.0]hexane is selected from bicifadine, enantiomers of bicifadine, salts of bicifadine, prodrugs of bicifadine, polymorphs, hydrates, and solvates of bicifadine, and combinations thereof.

18. The method of claim 17, wherein the 1-aryl-3-azabicyclo[3.1.0]hexane is bicifadine HCl.

19. A method for preventing or treating one or more symptom(s) resulting from a neuropathic disorder in a mammalian subject comprising administering to said subject an effective amount of a compound of formula V 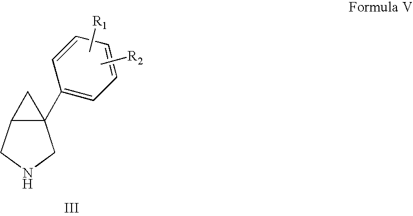
wherein R₁ and R₂ are each selected from halogen, C₁₋₃ alkyl, C₂₋₄ alkenyl, C₂₋₄ alkynyl, halo(C₁₋₃)alkyl, cyano, hydroxy, C₃₋₅ cycloalkyl, C₁₋₃ alkoxy, C₁₋₃ alkoxy(C₁₋₃)alkyl, carboxy(C₁₋₃)alkyl, C₁₋₃ alkanoyl, halo(C₁₋₃)alkoxy, nitro, amino, C₁₋₃ alkylamino, and di(C₁₋₃)alkylamino, methyl, ethyl, fluoro, chloro, trifluoromethyl, cyano, and trifluoromethoxy.

20. The method of claim 19, wherein the compound is selected from the group consisting of: 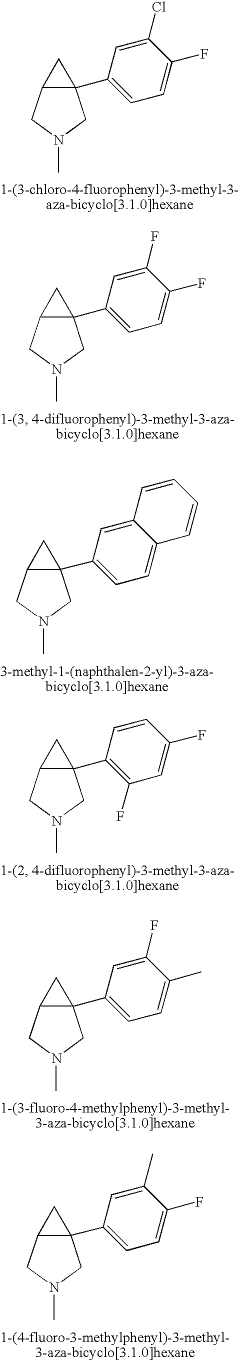 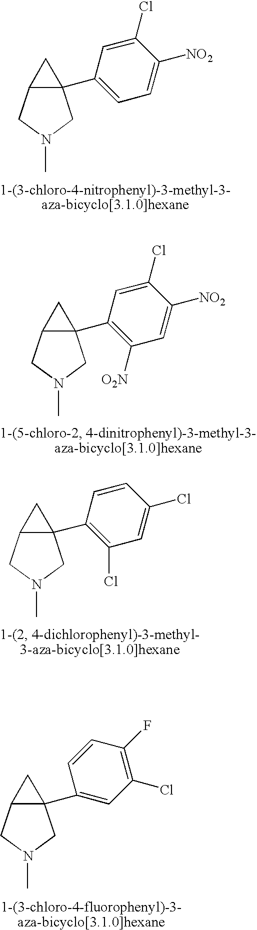
and pharmaceutically acceptable salts, enantiomers, polymorphs, solvates, hydrates and prodrugs thereof.

21-71. (canceled)
